(19) United States
(12) Patent Application Publication
(10) Pub. No.: US 2020/0102271 A1
(43) Pub. Date: Apr. 2, 2020

(54) ONIUM SALT, RESIST COMPOSITION, AND PATTERN FORMING PROCESS

(71) Applicant: Shin-Etsu Chemical Co., Ltd., Tokyo (JP)

(72) Inventors: Masaki Ohashi, Joetsu-shi (JP); Kazuya Honda, Joetsu-shi (JP); Yuki Suka, Joetsu-shi (JP); Yuki Kera, Joetsu-shi (JP)

(73) Assignee: Shin-Etsu Chemical Co., Ltd., Tokyo (JP)

(21) Appl. No.: 16/584,181

(22) Filed: Sep. 26, 2019

(30) Foreign Application Priority Data
Sep. 28, 2018 (JP) 2018-183103

(51) Int. Cl. C07C 381/12; G03F 7/029; G03F 7/20

(52) U.S. Cl. CPC C07C 381/12; G03F 7/70025; G03F 7/2053; G03F 7/029

(57) ABSTRACT

A novel onium salt having formula (1) and a resist composition comprising the same as a quencher are provided. When the resist composition is processed by photolithography using high-energy radiation, there is formed a resist pattern which is improved in LWR and CDU. In formula (1), R¹, R² and R³ each are a C₁-C₂₀ monovalent hydrocarbon group which may contain a heteroatom exclusive of fluorine, and Z⁺ is a sulfonium, iodonium or ammonium cation.

### CROSS-REFERENCE TO RELATED APPLICATION

This non-provisional application claims priority under 35 U.S.C. § 119(a) on Patent Application No. 2018-183103 filed in Japan on Sep. 28, 2018, the entire contents of which are hereby incorporated by reference.

### TECHNICAL FIELD

This invention relates to an onium salt, a resist composition comprising the same, and a pattern forming process using the resist composition.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic devices at the 65-nm node by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, extreme ultraviolet (EUV) lithography of 13.5 nm wavelength, and double patterning version of the ArF lithography, on which active research efforts have been made.

With the rapid progress toward miniaturization, it becomes difficult to form a pattern of desired size from prior art resist materials. In particular, the influence of acid diffusion is detrimental to lithography performance. For example, as the pattern size is approaching the diffusion length of acid, the degradation of contrast becomes more serious. As the mask error factor (MEF), indicative of a dimensional shift on wafer relative to a dimensional shift on mask, increases, a noticeable drop of mask fidelity ensues. In addition, the fluctuation of pattern line width, known as line width roughness (LWR), and the critical dimension uniformity (CDU) of pattern are largely affected by the acid diffusion. The degradation of these parameters becomes a problem.

To solve the outstanding problems, studies are made not only on base resins and photoacid generators, but also on diffusion controlling agents. A high level of acid diffusion control is effective for improving LWR and CDU. Amines are typically used as the diffusion controlling agent. Many problems associated with line width roughness (LWR) as an index of pattern roughness are left unsolved. Also the use of weak acid onium salts as the diffusion controlling agent is under study. For example, Patent Document 1 describes a positive photosensitive composition for ArF excimer laser lithography comprising a carboxylic acid onium salt. The composition is based on the mechanism that a salt exchange occurs between a weak acid onium salt and a strong acid (sulfonic acid) generated by another PAG upon exposure, to form a weak acid and a strong acid onium salt. That is, the strong acid (α,α-difluorosulfonic acid) having high acidity is replaced by a weak acid (alkanesulfonic acid or carboxylic acid), thereby suppressing acid-aided decomposition reaction of acid labile groups and reducing or controlling the distance of acid diffusion.

The onium salt apparently functions as a quencher, that is, diffusion controlling agent.

Such onium salt type quenchers are effective for improving LWR and CDU to some extent, but still unsatisfactory in the formation of narrower pitch patterns as required in the advanced miniaturization technology,

### CITATION LIST

Patent Document 1: JP 4226803 (U.S. Pat. No. 6,492,091)

### DISCLOSURE OF INVENTION

An object of the invention is to provide an onium salt and a resist composition comprising the same, which composition is processed by high-energy radiation lithography to form a resist pattern with improved LWR and CDU, and a pattern forming process using the resist composition.

The inventors have found that a resist composition comprising an onium salt of specific structure as a quencher can be processed by lithography to form a resist pattern with improved LWR and CDU, and is suited for high accuracy micropatterning.

In one aspect, the invention provides an onium salt having the formula (1).

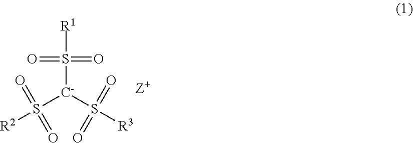

Herein R¹, R² and R³ are each independently a C₁-C₂₀ straight, branched or cyclic monovalent hydrocarbon group which may contain a heteroatom exclusive of fluorine, R² and R³ may bond together to form a ring with the sulfur atoms to which they are attached and the carbon atom in the formula, and Z⁺ is a sulfonium, iodonium or ammonium cation.

In another aspect, the invention provides a resist composition comprising (A) the onium salt defined above, (B) an organic solvent, (C) a polymer comprising recurring units containing an acid labile group, and (D) a photoacid generator.

In a preferred embodiment, the recurring units containing an acid labile group have the formula (a1) or (a2).

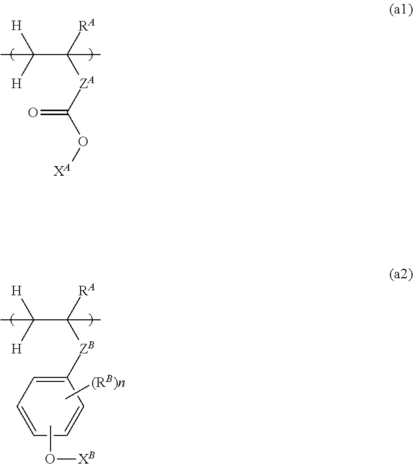

Herein R^{A} is each independently hydrogen, fluorine, methyl or trifluoromethyl, Z^{A} is a single bond, phenylene, naphthylene or (backbone)—C(═O)—O—Z^{A1}—, Z^{A1} is a C₁-C₁₀ straight, branched or cyclic alkanediyl group which may contain a hydroxyl moiety, ether bond, ester bond or lactone ring, or phenylene or naphthylene group, Z^{B} is a single bond or (backbone)—C(═O)—O—, X^{A} and X^{B} are each independently an acid labile group, R^{B} is a C₁-C₂₀ straight, branched or cyclic monovalent hydrocarbon group which may contain a heteroatom, and n is an integer of 0 to 4.

In a preferred embodiment, the polymer further comprises recurring units having the formula (b1) or (b2).

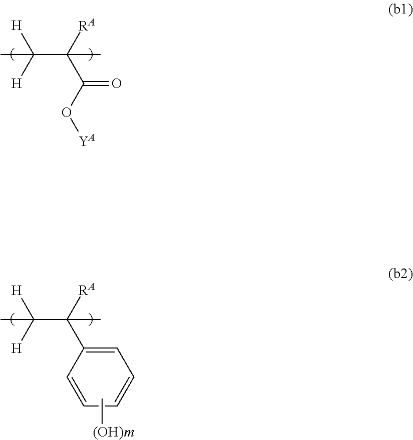

Herein R^{A} is as defined above, Y^{A} is hydrogen or a polar group containing at least one structure selected from the group consisting of hydroxyl, cyano, carbonyl, carboxyl, ether bond, ester bond, sulfonic acid ester bond, carbonate bond, lactone ring, sultone ring and carboxylic anhydride, and m is 1 or 2.

In a preferred embodiment, the photoacid generator (D) has the formula (2) or (3).

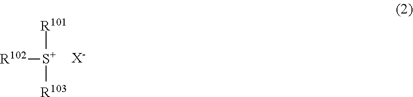

Herein R¹⁰¹, R¹⁰² and R¹⁰³ are each independently a C₁-C₂₀ straight, branched or cyclic monovalent hydrocarbon group which may contain a heteroatom, any two of R¹⁰¹, R¹⁰² and R¹⁰³ may bond together to form a ring with the sulfur atom to which they are attached, and X⁻ is an anion selected from the following formulae (2A) to (2D):

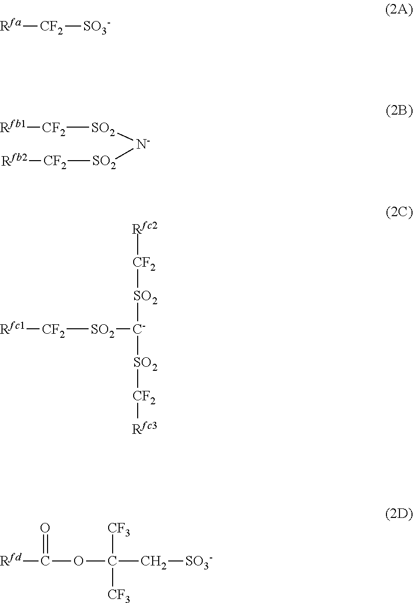

wherein R^{fa}, R^{fb1}, R^{fb2}, R^{fc1}, R^{fc2} and R^{fc3} are each independently fluorine or a C₁-C₄₀ straight, branched or cyclic monovalent hydrocarbon group which may contain a heteroatom, or a pair of R^{fb1} and R^{fb2}, or R^{fc1} and R^{fc2} may bond together to form a ring with the carbon atom to which they are attached and any intervening atoms, R^{fd} is a C₁-C₄₀ straight, branched or cyclic monovalent hydrocarbon group which may contain a heteroatom.

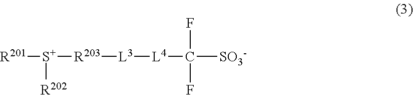

Herein R²⁰¹ and R²⁰² are each independently a C₁-C₂₀ straight, branched or cyclic monovalent hydrocarbon group which may contain a heteroatom, R²⁰³ is a C₁-C₂₀ straight, branched or cyclic divalent hydrocarbon group which may contain a heteroatom, any two of R²⁰¹, R²⁰² and R²⁰³ may bond together to form a ring with the sulfur atom to which they are attached, L³ is a divalent linking group, and L⁴ is a single bond or a C₁-C₂₀ straight, branched or cyclic divalent hydrocarbon group which may contain a heteroatom.

The resist composition may further comprise (E) a surfactant which is insoluble or substantially insoluble in water and soluble in alkaline developer, and/or a surfactant which is insoluble or substantially insoluble in water and alkaline developer, and/or (F) a nitrogen-containing compound.

In a further aspect, the invention provides a pattern forming process comprising the steps of applying the resist composition defined above onto a substrate to form a resist film, exposing the resist film to KrF excimer laser, ArF excimer laser, EB or EUV, and developing the exposed resist film in a developer.

### Advantageous Effects of Invention

Since the onium salt carries out a satisfactory function of acid diffusion controlling agent or quencher in a resist composition, the inventive resist composition comprising the same enables to form a pattern of good profile with a high resolution and improved LWR, CDU and focus margin.

### BRIEF DESCRIPTION OF ACCOMPANYING DRAWINGS

FIG. 1 is a diagram of ¹H-NMR spectrum of Compound Q-A obtained in Example 1-1.

FIG. 2 is a diagram of ¹H-NMR spectrum of Compound Q-B obtained in Example 1-2.

FIG. 3 is a diagram of ¹H-NMR spectrum of Compound Q-C obtained in Example 1-3.

FIG. 4 is a diagram of ¹H-NMR spectrum of Compound Q-D obtained in Example 1-4.

FIG. 5 is a diagram of ¹H-NMR spectrum of Compound Q-E obtained in Example 1-5.

### DESCRIPTION OF EMBODIMENTS

As used herein, the singular forms “a,” “an” and “the” include plural referents unless the context clearly dictates otherwise. The notation (Cn-Cm) means a group containing from n to m carbon atoms per group.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity
MEF: mask error factor
DOF: depth of focus

In structural formulae, the broken line denotes a valence bond; Me stands for methyl, Ph for phenyl, and Ac for acetyl.

### Onium Salt

One embodiment of the invention is an onium salt having the formula (1).

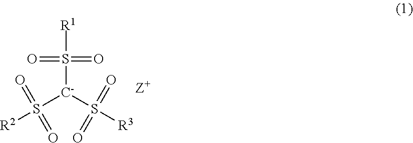

In formula (1), R¹, R² and R³ are each independently a C₁-C₂₀ straight, branched or cyclic monovalent hydrocarbon group which may contain a heteroatom exclusive of fluorine. R² and R³ may bond together to form a ring with the sulfur atoms to which they are attached and the carbon atom in the formula.

Suitable monovalent hydrocarbon groups include alkyl groups such as methyl, ethyl, n-propyl, isopropyl, n-butyl, sec-butyl, tert-butyl, tert-pentyl, n-pentyl, n-hexyl, n-octyl, n-nonyl, n-decyl, cyclopentyl, cyclohexyl, 2-ethylhexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl, and aryl groups such as phenyl, naphthyl, and anthracenyl. In these groups, some hydrogen may be substituted by a moiety containing a heteroatom such as oxygen, sulfur, nitrogen, or halogen exclusive of fluorine, or a moiety containing a heteroatom such as oxygen, sulfur or nitrogen may intervene between carbon atoms, so that the group may contain a hydroxyl moiety, cyano moiety, carbonyl moiety, ether bond, thioether bond, ester bond, sulfonate (sulfonic acid ester) bond, carbonate bond, carbamate bond, lactone ring, sultone ring, or carboxylic anhydride (—C(═O)—O—C(═O)—).

Inter alia, R¹, R² and R³ each are preferably a C₁-C₂₀ alkyl or C₆-C₂₀ aryl group which may contain a heteroatom exclusive of fluorine.

Illustrative structures of the anion moiety in the onium salt having formula (1) are shown below, but not limited thereto.

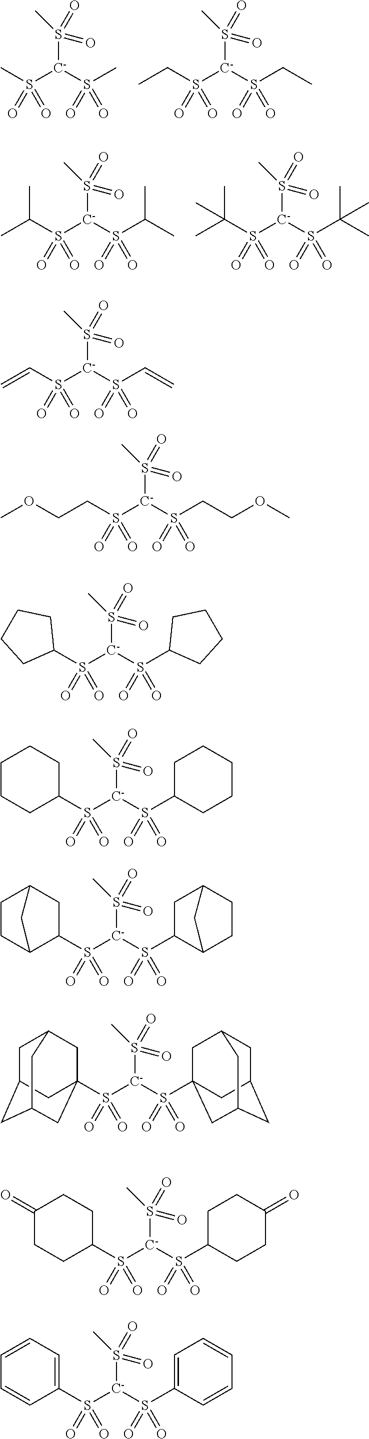

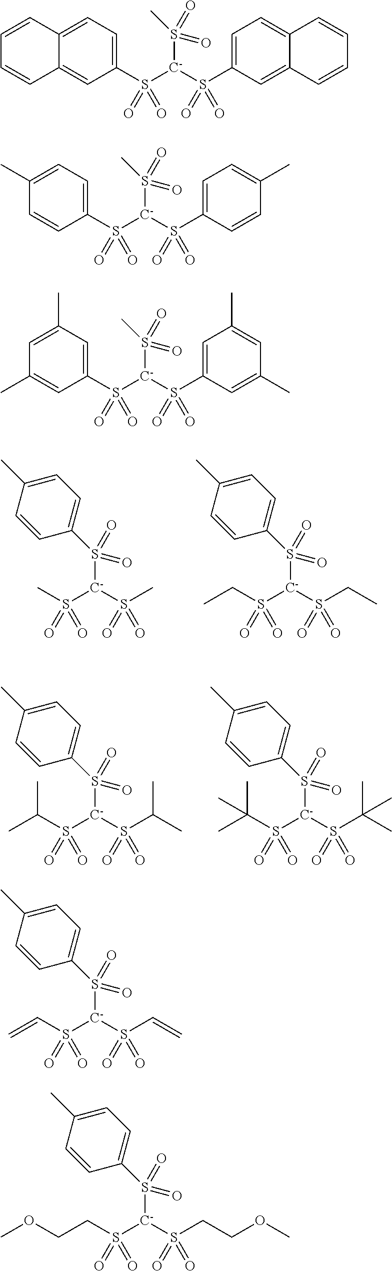

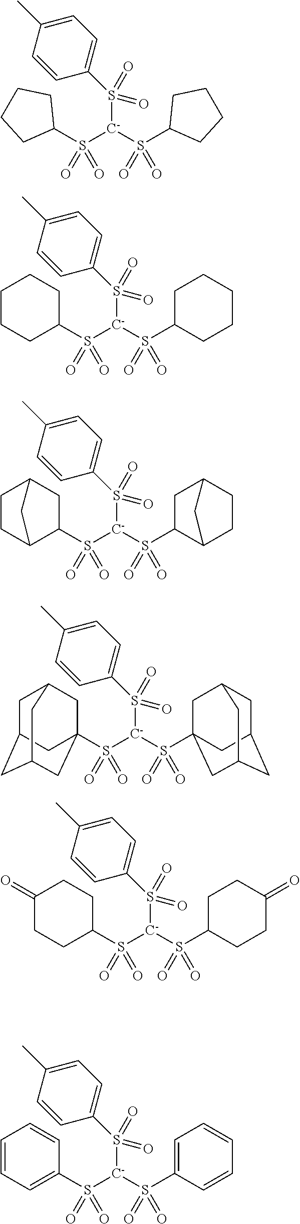

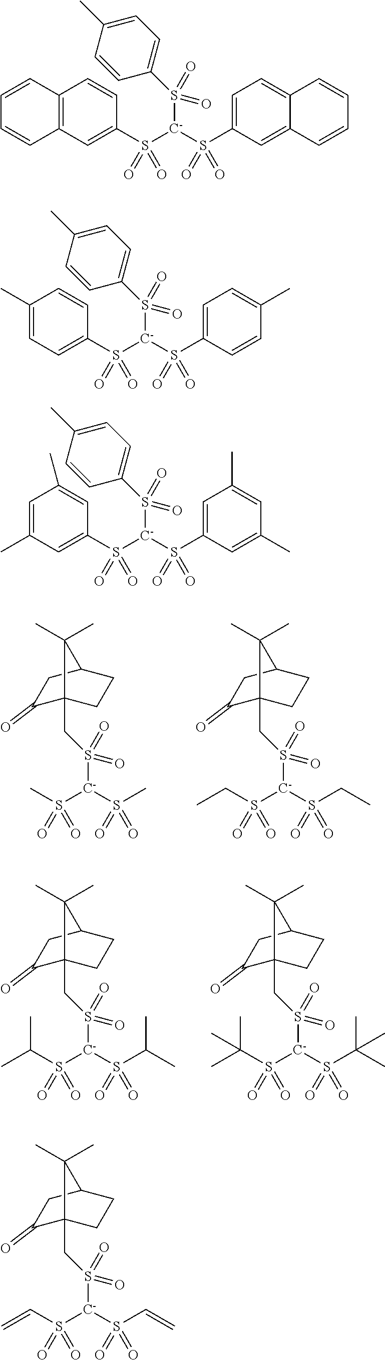

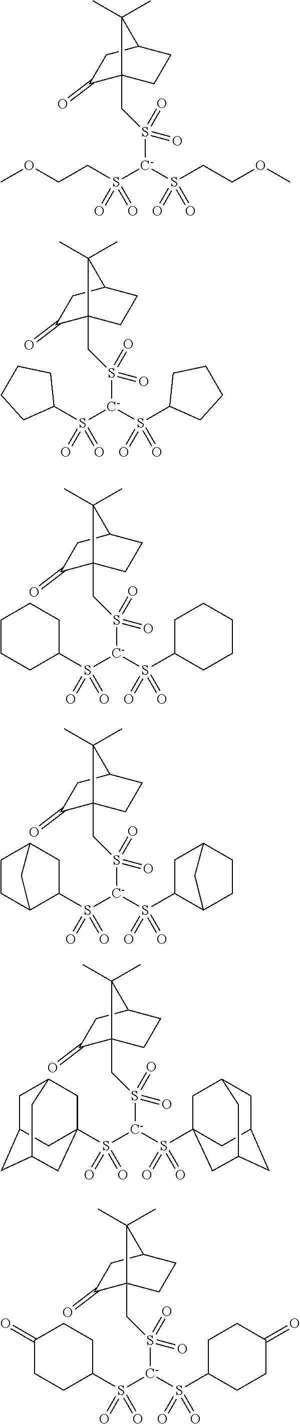

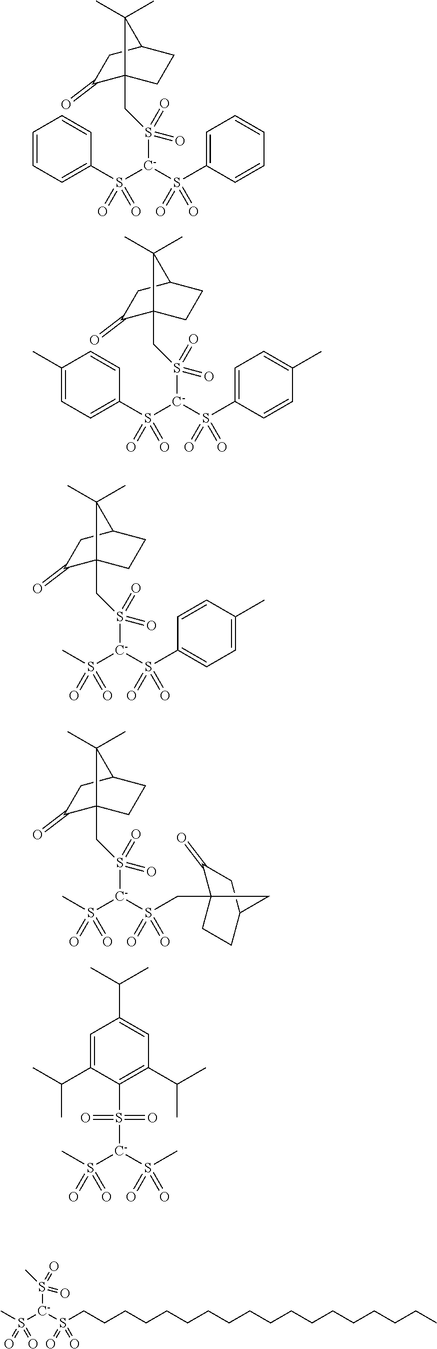

In formula (1), Z⁺ is a sulfonium, iodonium or ammonium cation. The preferred sulfonium, iodonium and ammonium cations have the following formulae (1-1), (1-2) and (1-3), respectively.

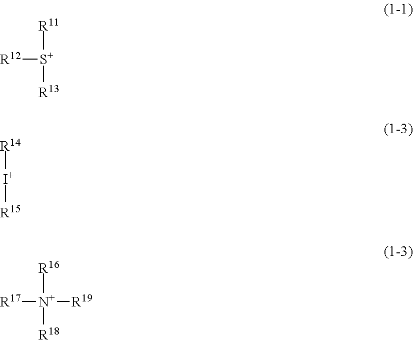

Herein R¹¹ to R¹⁹ are each independently a C₁-C₂₀ straight, branched or cyclic monovalent hydrocarbon group which may contain a heteroatom. In formula (1-1), any two of R¹¹ to R¹³ may bond together to form a ring with the sulfur atom to which they are attached. In formula (1-3), any two or more of R¹⁶ to R¹⁹ may bond together to form a ring with the nitrogen atom to which they are attached.

Suitable monovalent hydrocarbon groups include alkyl groups such as methyl, ethyl, propyl, isopropyl, n-butyl, sec-butyl, tert-butyl, tert-pentyl, n-pentyl, n-hexyl, n-octyl, n-nonyl, n-decyl, cyclopentyl, cyclohexyl, 2-ethylhexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, adamantylmethyl, and aryl groups such as phenyl, naphthyl, and anthracenyl. In these groups, some hydrogen may be substituted by a moiety containing a heteroatom such as oxygen, sulfur, nitrogen, or halogen, or a moiety containing a heteroatom such as oxygen, sulfur or nitrogen may intervene between carbon atoms, so that the group may contain a hydroxyl moiety, cyano moiety, carbonyl moiety, ether bond, thioether bond, ester bond, sulfonate bond, carbonate bond, carbamate bond, lactone ring, sultone ring, carboxylic anhydride, or haloalkyl moiety.

Examples of the sulfonium cation include triphenylsulfonium, 4-hydroxyphenyldiphenylsulfonium, bis(4-hydroxyphenyl)phenylsulfonium, tris(4-hydroxyphenyl)sulfonium, 4-tert-butylphenyldiphenylsulfonium, bis(4-tert-butoxyphenyl)phenylsulfonium, tris(4-tert-butylphenyl)sulfonium, tris(4-tert-butoxyphenyl)sulfonium, 3-tert-butoxyphenyldiphenylsulfonium, bis(3-tert-butoxyphenyl)phenylsulfonium, tris(3-tert-butoxyphenyl)sulfonium, 3,4-di-tert-butoxyphenyldiphenylsulfonium, bis(3,4-di-tert-butoxyphenyl)phenylsulfonium, tris(3,4-di-tert-butoxyphenyl)sulfonium, diphenyl(4-thiophenoxyphenyl)sulfonium, 4-tert-butoxycarbonylmethyloxyphenyldiphenylsulfonium, tris(4-tert-butoxycarbonylmethyloxyphenyl)sulfonium, (4-tert-butoxyphenyl)bis(4-dimethylaminophenyl)sulfonium, tris(4-dimethylaminophenyl)sulfonium, 2-naphthyldiphenylsulfonium, (4-hydroxy-3,5-dimethylphenyl)diphenylsulfonium, (4-n-hexyloxy-3,5-dimethylphenyl)diphenylsulfonium, dimethyl(2-naphthyl)sulfonium, 4-hydroxyphenyldimethylsulfonium, 4-methoxyphenyldimethylsulfonium, trimethylsulfonium, 2-oxocyclohexylcyclohexylmethylsulfonium, trinaphthylsulfonium, tribenzylsulfonium, diphenylmethylsulfonium, dimethylphenylsulfonium, 2-oxo-2-phenylethylthiacyclopentanium, diphenyl-2-thienylsulfonium, 4-n-butoxynaphthyl-1-thiacyclopentanium, 2-n-butoxynaphthyl-1-thiacyclopentanium, 4-methoxynaphthyl-1-thiacyclopentanium, and 2-methoxynaphthyl-1-thiacyclopentanium. Of these, triphenylsulfonium, 4-tert-butylphenyldiphenylsulfonium, 4-tert-butoxyphenyldiphenylsulfonium, tris(4-tert-butylphenyl)sulfonium, tris(4-tert-butoxyphenyl)sulfonium, and dimethylphenylsulfonium are more preferred.

Further examples of the sulfonium cation include those of the following formulae.

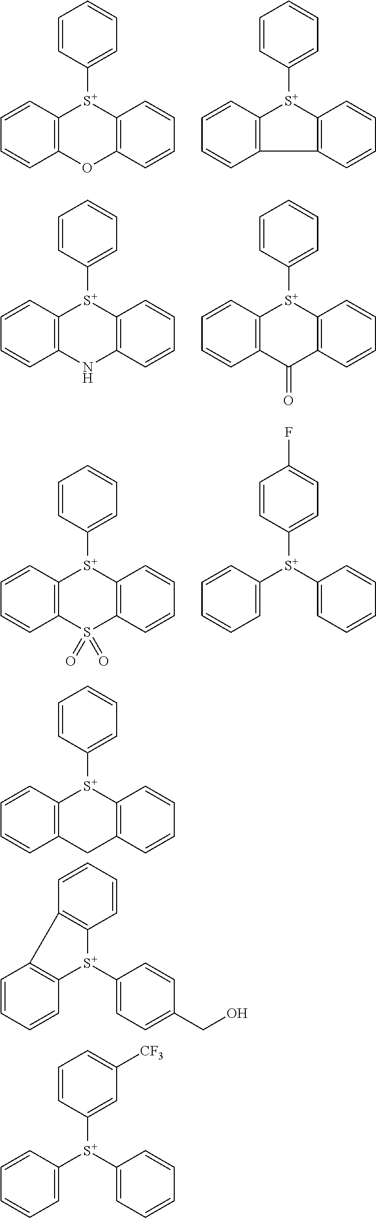

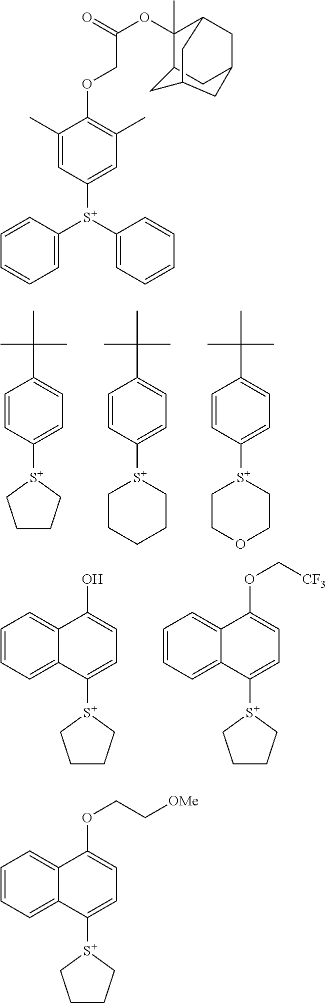

Examples of the iodonium cation include diphenyliodonium, bis(4-methylphenyl)iodonium, bis(4-ethylphenyl)iodonium, bis(4-tert-butylphenyl)iodonium, bis(4-(1,1-dimethylpropyl)phenyl)iodonium, bis(4-methoxyphenyl)iodonium, 4-methoxyphenylphenyliodonium, 4-tert-butoxyphenylphenyliodonium, 4-acryloyloxyphenylphenyliodonium, 4-methacryloyloxyphenylphenyliodonium, and 4-fluorophenylphenyliodonium.

Examples of the ammonium cation are shown below.

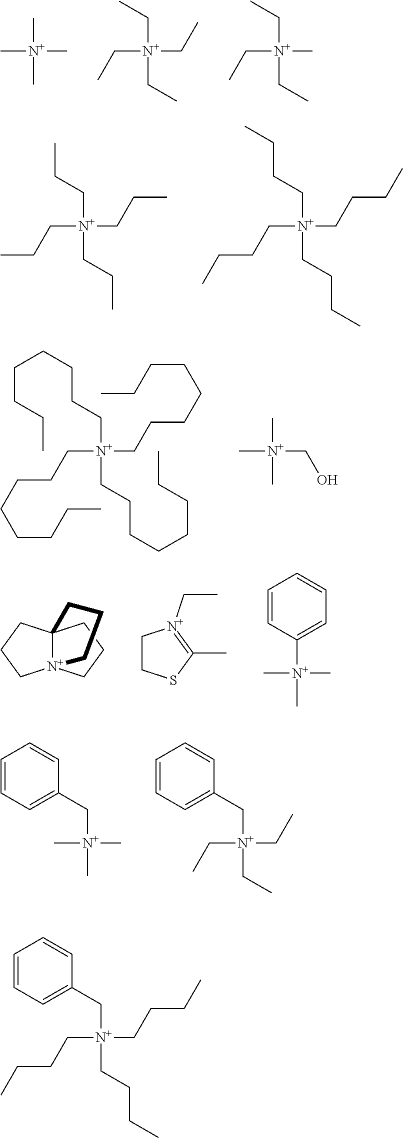

Exemplary structures of the onium salt having formula (1) include arbitrary combinations of anions with cations, both as exemplified above. Preferred are combinations of anions with sulfonium cations, both as exemplified above.

The inventive onium salt may be synthesized by a combination of known organic chemistry methods, for example, according to the scheme shown below.

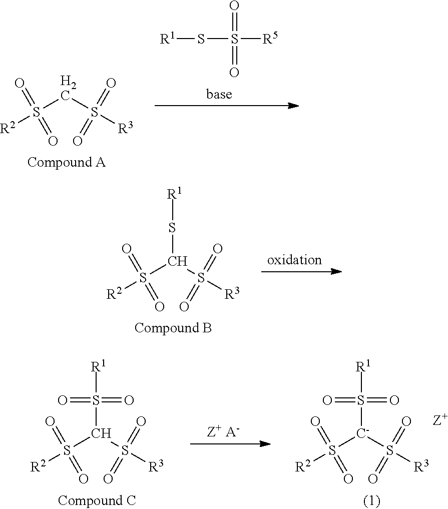

Herein R¹, R², R³ and Z⁺ are as defined above. R⁵ is a C₁-C₂₀ straight, branched or cyclic monovalent hydrocarbon group which may contain a heteroatom, examples of which are as exemplified above for R¹, R², and R³. A⁻ is an anion, for example, chloride, bromide, iodide, hydrogencarbonate, methanesulfonate, tosylate, methylsulfate or triflate ion.

The first step is S-alkylation of Compound A to Compound B. Typical of the S-alkylating agent is S-methyl thiomethanesulfonate. The second step is to oxidize Compound B to form Compound C or trisalkanesulfonylmethide acid. The oxidation reaction may be performed by a known organic chemistry method. The subsequent third step is ion exchange reaction of Compound C with an onium salt intermediate having the desired cation (Z⁺A⁻) to synthesize the target onium salt having formula (1). The ion exchange reaction may be performed by a well-known method, for example, according to the teaching of JP-A 2007-145797.

The inventive onium salt functions as a quite effective quencher when it is applied to a resist composition. As used herein, the quencher is a compound capable of trapping an acid generated by a PAG in a resist composition and preventing the acid from diffusing to the unexposed region, for thereby forming the desired pattern.

The acid diffusion controlling mechanism of the onium salt is estimated as follows.

In a resist composition, a PAG generates an acid which must be strongly acidic enough to deprotect the acid labile group on the base resin, for example, a sulfonic acid which is fluorinated at ϵ-position relative to sulfo group, imide acid (imidic acid) or methide acid in the case of ArF lithography. If a PAG and the inventive onium salt are co-present in a resist composition, the acid generated by the PAG is trapped by salt exchange reaction with the inventive onium salt. Via the salt exchange, trisalkanesulfonyl methide acid is released from the inventive onium salt, which has an acid strength insufficient to deprotect the acid labile groups on the base resin. Accordingly, the inventive onium salt functions as a quencher for trapping the acid generated by the PAG. It is noted that analogous structures are known from the past, for example, onium salts, especially sulfonium salts of tris(trifluoromethanesulfonyl)methide are known from U.S. Pat. No. 5,554,664. Since these compounds have a very high acidity due to the electron attractive effect of fluorine atoms, they does not function as a quencher, but as a photoacid generator capable of readily cleaving acid labile groups on the base resin. In contrast, the anion moiety of the inventive onium salt is a fluorine-free methide anion and forms a conjugated base of weak acid, which has significantly different physical properties.

In general, the onium salt type quenchers tend to form a resist pattern with better CDU and LWR than the amine compound quenchers. The reason is presumed as follows. Salt exchange between strong acid and the inventive onium salt is repeated infinitely. The site where strong acid is generated at the end of exposure is different from the initial site where the strong acid-generating onium salt is present. Since the cycle of photo-acid generation and salt exchange is repeated over and over, the acid generation points are averaged. Due to this smoothing effect, a resist pattern after development is improved in CDU and LWR.

Materials capable of exerting a quencher effect by a similar-mechanism are known. For example, Patent Document 1 and JP-A 2003-005376 use onium salts of carboxylic acids, alkanesulfonic acids and arylsulfonic acids as the quencher. JP-A 2012-108447 describes a quencher of sulfonamide type. The quenchers reported thus far fail to meet the desired performance in the further miniaturized region. By contrast, the inventive onium salt achieves an excellent resist performance even in forming a narrow pitch fine pattern. Although the reason is not well understood, it is believed that the performance is ascribed to the structural specificity of the inventive onium salt. For example, since an onium salt of carboxylic acid or alkanesulfonic acid is highly polar and thus less soluble in an organic solvent, there is a possibility that the onium salt agglomerates locally in a resist film. In contrast, the inventive onium salt is so soluble in an organic solvent that it is uniformly dispersed in a resist film. Also, the anion of the inventive onium salt has a methide acid structure and is thus low nucleophilic as compared with the onium salt of carboxylic acid or sulfonic acid. Thus the inventive onium salt eliminates the risk of reacting with other components and has good stability. Additionally, because of an adequate acidity, the inventive onium salt causes no cleavage of acid labile groups on the base resin. The inventive onium salt functions as a quite effective quencher, eventually improving resist performance, especially in terms of CDU and LWR.

Resist Composition Another embodiment of the invention is directed to a resist composition comprising (A) a quencher in the form of the onium salt having formula (1) as an essential component, (B) an organic solvent, (C) a polymer comprising recurring units containing an acid labile group (base resin), and (D) a photoacid generator as optional components. If necessary, the resist composition may further comprise (E) a surfactant which is insoluble or substantially insoluble in water and soluble in alkaline developer, and/or a surfactant which is insoluble or substantially insoluble in water and alkaline developer (hydrophobic resin) and (F) a nitrogen-containing component.

The amount of the quencher (A) blended is preferably 0.1 to 40 parts by weight, more preferably 1 to 20 parts by weight per 100 parts by weight of the base resin (C). As long as the amount of the quencher is within the range, a satisfactory quencher function is exerted, and any performance losses such as a lowering of sensitivity and formation of foreign particles due to a shortage of dissolution are avoided. The onium salt may be used alone or in admixture as the quencher (A).

### (B) Organic Solvent

Any organic solvent may be used as long as components are soluble therein. Examples of the organic solvent are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol and diacetone alcohol; ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone, and mixtures thereof. Where an acid labile group of acetal form is used, a high-boiling alcohol solvent such as diethylene glycol, propylene glycol, glycerol, 1,4-butanediol or 1,3-butanediol may be added for accelerating deprotection reaction of acetal. Of the above organic solvents, it is recommended to use 1-ethoxy-2-propanol, PGMEA, cyclohexanone, γ-butyrolactone, and mixtures thereof.

An appropriate amount of the organic solvent (B) used is 200 to 5,000 parts, more preferably 400 to 3,000 parts by weight per 100 parts by weight of the base resin (C).

### (C) Base Resin

The base resin used in the resist composition is preferably a polymer comprising recurring units containing an acid labile group, preferably recurring units having the formula (a1) or (a2).

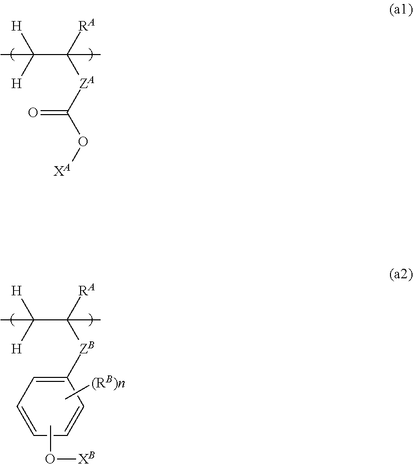

In formulae (a1) and (a2), R^{A} is each independently hydrogen, fluorine, methyl or trifluoromethyl. Z^{A} is a single bond, phenylene, naphthylene or (backbone)—C(═O)—O—Z^{A1}—, wherein Z^{A1} is a C₁-C₁₀ straight, branched or cyclic alkanediyl group which may contain a hydroxyl moiety, ether bond, ester bond or lactone ring, or a phenylene or naphthylene group. Z^{B} is a single bond or (backbone)—C(═O)—O—. X^{A} and X^{B} are each independently an acid labile group. R^{B} is a C₁-C₂₀ straight, branched or cyclic monovalent hydrocarbon group which may contain a heteroatom, and n is an integer of 0 to 4.

Examples of the structure of formula (a1) wherein Z^{A} is a variant are illustrated below, but not limited thereto. Herein R^{A} and X^{A} are as defined above.

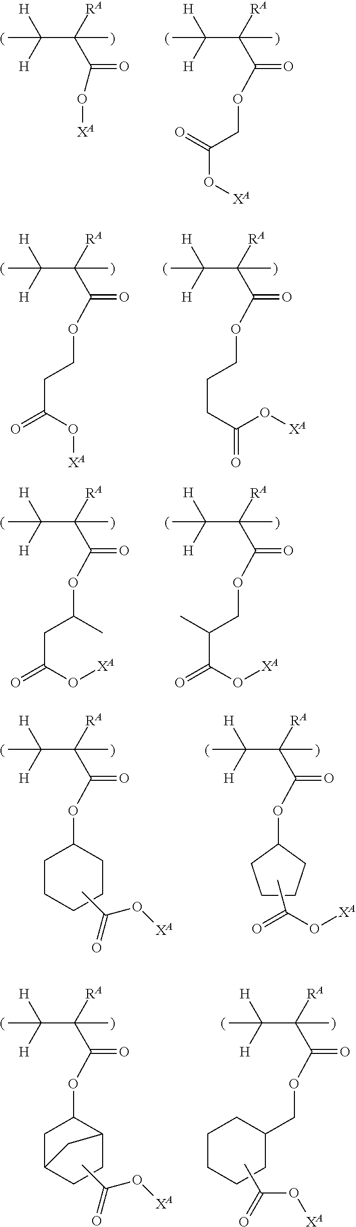

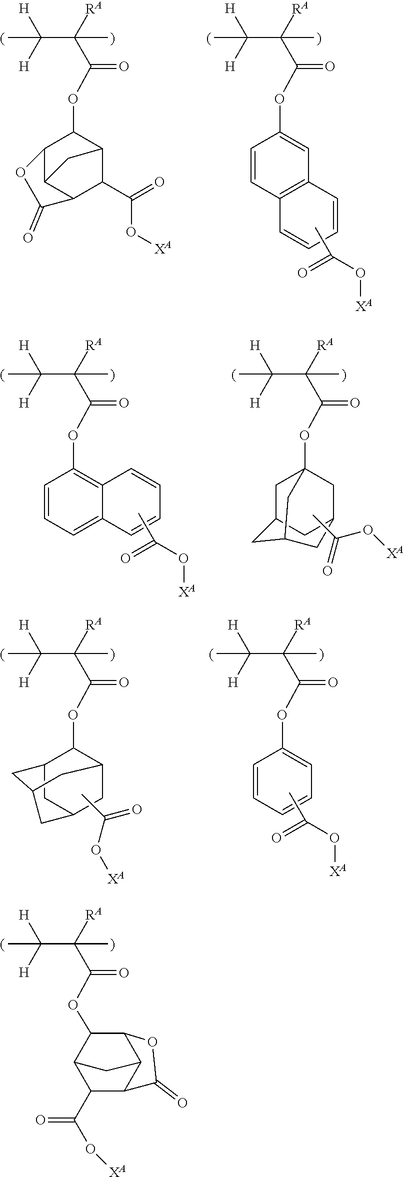

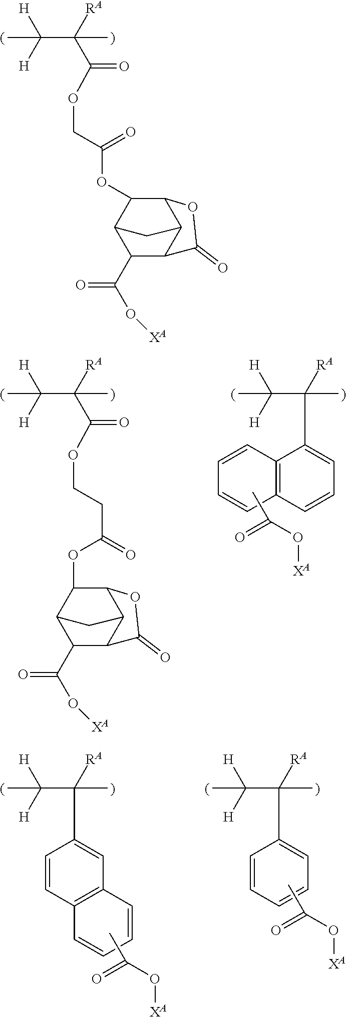

A polymer comprising recurring units having formula (a1) is decomposed under the action of acid to generate a carboxyl group and thus turns alkali soluble.

The acid labile group represented by X^{A} and X^{B} may be selected from a variety of such groups. Examples of the acid labile group include groups of the following formulae (L1) to (L4), tertiary alkyl groups of 4 to 20 carbon atoms, preferably 4 to 15 carbon atoms, trialkylsilyl groups in which each alkyl moiety has 1 to 6 carbon atoms, and oxoalkyl groups of 4 to 20 carbon atoms.

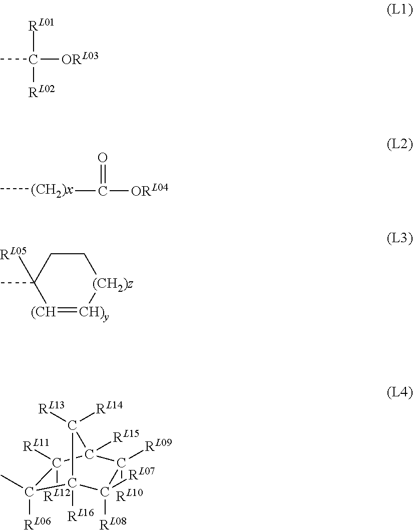

In formula (L1), R^{L01} and R^{L02} are hydrogen or straight, branched or cyclic alkyl groups of 1 to 18 carbon atoms, preferably 1 to 10 carbon atoms. Exemplary alkyl groups include methyl, ethyl, propyl, isopropyl, n-butyl, sec-butyl, tert-butyl, cyclopentyl, cyclohexyl, 2-ethylhexyl, n-octyl, norbornyl, tricyclodecanyl, tetracyclododecanyl, and adamantyl.

R^{L03} is a monovalent hydrocarbon group of 1 to 18 carbon atoms, preferably 1 to 10 carbon atoms, which may contain a moiety containing a heteroatom such as oxygen. Examples of the monovalent hydrocarbon group include straight, branched or cyclic alkyl groups and substituted forms thereof in which some hydrogen is substituted by hydroxyl, alkoxy, oxo, amino, alkylamino or the like, or in which some carbon is replaced by a moiety containing a heteroatom such as oxygen. Suitable alkyl groups are as exemplified above for R^{L01} and R^{L02}. Illustrative examples of the substituted alkyl groups are shown below.

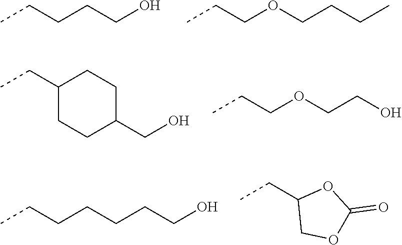

A pair of R^{L01} and R^{L02}, R^{L01} and R^{L03}, or R^{L02} and R^{L03} may bond together to form a ring with the carbon and oxygen atom to which they are attached. Each of R^{L01}, R^{L02} and R^{L03} is a straight or branched alkanediyl group of 1 to 18 carbon atoms, preferably 1 to 10 carbon atoms when they form a ring.

In formula (L2), R^{L04} is a tertiary alkyl group of 4 to 20 carbon atoms, preferably 4 to 15 carbon atoms, a trialkylsilyl group in which each alkyl moiety has 1 to 6 carbon atoms, an oxoalkyl group of 4 to 20 carbon atoms, or a group of formula (L1). Exemplary tertiary alkyl groups are tert-butyl, tert-pentyl, 1,1-diethylpropyl, 2-cyclopentylpropan-2-yl, 2-cyclohexylpropan-2-yl, 2-(bicyclo[2.2.1]heptan-2-yl)propan-2-yl, 2-(adamantan-1-yl)propan-2-yl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, 2-methyl-2-adamantyl, and 2-ethyl-2-adamantyl. Exemplary trialkylsilyl groups are trimethylsilyl, triethylsilyl, and dimethyl-tert-butylsilyl. Exemplary oxoalkyl groups are 3-oxocyclohexyl, 4-methyl-2-oxooxan-4-yl, and 5-methyl-2-oxooxolan-5-yl. Letter x is an integer of 0 to 6.

In formula (L3), R^{L05} is an optionally substituted, straight, branched or cyclic C₁-C₈ alkyl group or an optionally substituted C₆-C₂₀ aryl group. Examples of the optionally substituted alkyl group include straight, branched or cyclic alkyl groups such as methyl, ethyl, propyl, isopropyl, n-butyl, sec-butyl, tert-butyl, tert-pentyl, n-pentyl, n-hexyl, cyclopentyl and cyclohexyl, and substituted forms of the foregoing in which some hydrogen is substituted by hydroxyl, alkoxy, carboxyl, alkoxycarbonyl, oxo, amino, alkylamino, cyano, mercapto, alkylthio, sulfo or the like. Examples of the optionally substituted aryl groups include phenyl, methylphenyl, naphthyl, anthryl, phenanthryl, and pyrenyl, and substituted forms of the foregoing in which some hydrogen is substituted by hydroxyl, alkoxy, carboxyl, alkoxycarbonyl, oxo, amino, alkylamino, cyano, mercapto, alkylthio, sulfo or the like. Letter y is equal to 0 or 1, z is an integer of 0 to 3, and 2y+z is equal to 2 or 3.

In formula (L4), R^{L06} is an optionally substituted, straight, branched or cyclic C₁-C₈ alkyl group or an optionally substituted C₆-C₂₀ aryl group. Examples of the alkyl and aryl groups are the same as exemplified for R^{L05}.

R^{L07} to R^{L16} are each independently hydrogen or a C₁-C₁₅ monovalent hydrocarbon group. Suitable hydrocarbon groups include straight, branched or cyclic alkyl groups such as methyl, ethyl, propyl, isopropyl, n-butyl, sec-butyl, tert-butyl, tert-pentyl, n-pentyl, n-hexyl, n-octyl, n-nonyl, n-decyl, cyclopentyl, cyclohexyl, cyclopentylmethyl, cyclopentylethyl, cyclopentylbutyl, cyclohexylmethyl, cyclohexylethyl and cyclohexylbutyl, and substituted forms of the foregoing in which some hydrogen is substituted by hydroxyl, alkoxy, carboxyl, alkoxycarbonyl, oxo, amino, alkylamino, cyano, mercapto, alkylthio, sulfo or the like. Alternatively, two of R^{L07} to R^{L16} may bond together to form a ring with the carbon atom to which they are attached (for example, a pair of R^{L07} and R^{L08}, R^{L07} and R^{L09}, R^{L07} and R^{L10}, R^{L08} and R^{L10}, R^{L09} and R^{L10}, R^{L11} and R^{L12}, R^{L13} and R^{L14}, or a similar pair form a ring). Each of R^{L07} to R^{L16} represents a divalent C₁-C₁₅ hydrocarbon group when they form a ring, examples of which are the ones exemplified above for the monovalent hydrocarbon groups, with one hydrogen atom being eliminated. Two of R^{L07} to R^{L16} which are attached to vicinal carbon atoms may bond together directly to form a double bond (for example, a pair of R^{L07} and R^{L09}, R^{L09} and R^{L15}, R^{L13} and R^{L15}, R^{L14} and R^{L15}, or a similar pair).

Of the acid labile groups of formula (L1), the straight and branched ones are exemplified by the following groups, but not limited thereto.

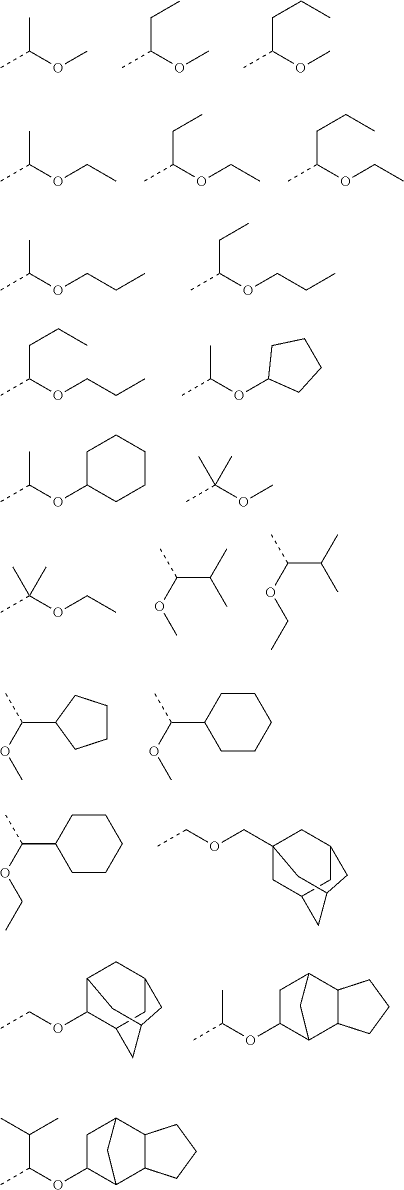

Of the acid labile groups of formula (L1), the cyclic ones are, for example, tetrahydrofuran-2-yl, 2-methyltetrahydrofuran-2-yl, tetrahydropyran-2-yl, and 2-methyltetrahydropyran-2-yl.

Examples of the acid labile groups of formula (L2) include tert-butoxycarbonyl, tert-butoxycarbonylmethyl, tert-pentyloxycarbonyl, tert-pentyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Examples of the acid labile groups of formula (L3) include 1-methylcyclopentyl, 1-ethylcyclopentyl, 1-n-propylcyclopentyl, 1-isopropylcyclopentyl, 1-n-butylcyclopentyl, 1-sec-butylcyclopentyl, 1-cyclohexylcyclopentyl, 1-(4-methoxy-n-butyl)cyclopentyl, 1-methylcyclohexyl, 1-ethylcyclohexyl, 3-methyl-1-cyclopenten-3-yl, 3-ethyl-1-cyclopenten-3-yl, 3-methyl-1-cyclohexen-3-yl, and 3-ethyl-1-cyclohexen-3-yl groups.

Of the acid labile groups having formula (L4), groups having the following formulas (L4-1) to (L4-4) are preferred.

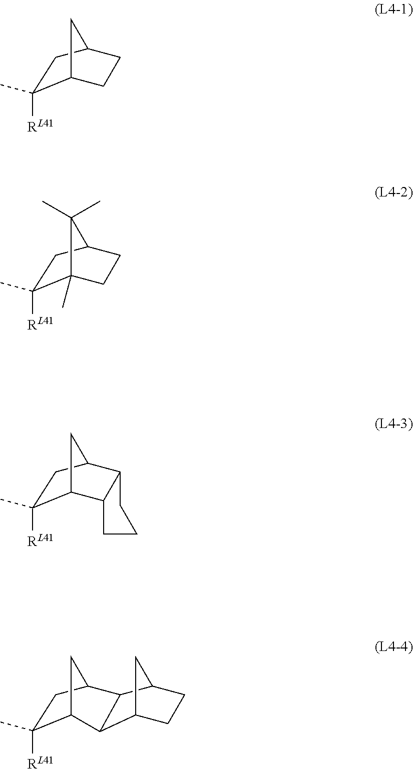

In formulas (L4-1) to (L4-4), the broken line denotes a bonding site and direction. R^{L41} is each independently a C₁-C₁₀ straight, branched or cyclic monovalent hydrocarbon group. Exemplary of the monovalent hydrocarbon group are alkyl groups such as methyl, ethyl, propyl, isopropyl, n-butyl, sec-butyl, tert-butyl, tert-pentyl, n-pentyl, n-hexyl, cyclopentyl and cyclohexyl.

For formulas (L4-1) to (L4-4), there can exist stereoisomers (enantiomers or diastereomers). Each of formulae (L4-1) to (L4-4) collectively represents all such stereoisomers. Such stereoisomers may be used alone or in admixture.

For example, the formula (L4-3) represents one or a mixture of two selected from groups having the following formulas (L4-3-1) and (L4-3-2).

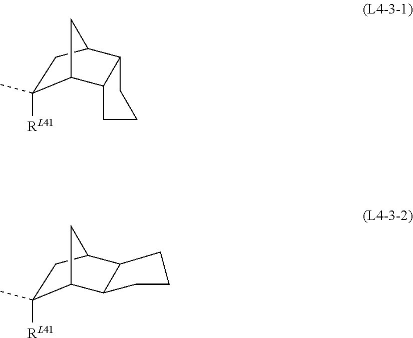

Herein R^{L41} is as defined above.

Similarly, the formula (L4-4) represents one or a mixture of two or more selected from groups having the following formulas (L4-4-1) to (L4-4-4).

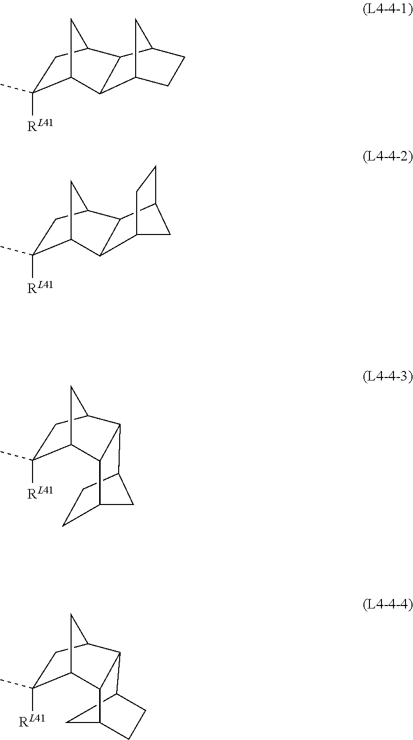

Herein R^{L41} is as defined above.

Each of formulas (L4-1) to (L4-4), (L4-3-1) and (L4-3-2), and (L4-4-1) to (L4-4-4) collectively represents an enantiomer thereof and a mixture of enantiomers.

It is noted that in the above formulas (L4-1) to (L4-4), (L4-3-1) and (L4-3-2), and (L4-4-1) to (L4-4-4), the bond direction is on the exo side relative to the bicyclo[2.2.1]heptane ring, which ensures high reactivity for acid catalyzed elimination reaction (see JP-A 2000-336121). In preparing these monomers having a tertiary exo-alkyl group of bicyclo[2.2.1]heptane skeleton as a substituent group, there may be contained monomers substituted with an endo-alkyl group as represented by the following formulas (L4-1-endo) to (L4-4-endo). For good reactivity, an exo proportion of at least 50 mol % is preferred, with an exo proportion of at least 80 mol % being more preferred.

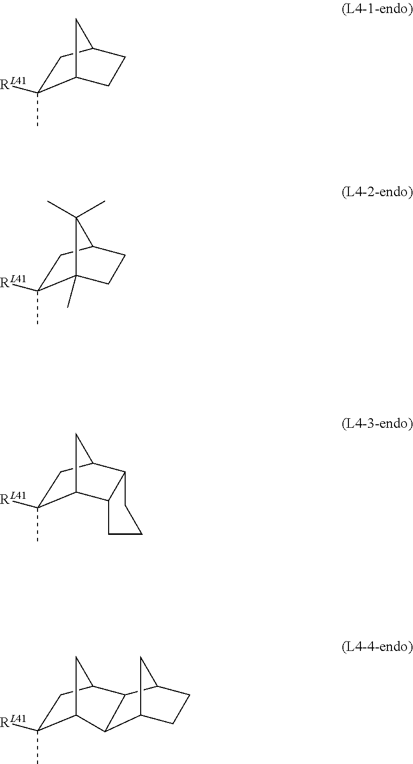

Herein R^{L41} is as defined above.

Illustrative examples of the acid labile group of formula (L4) are given below.

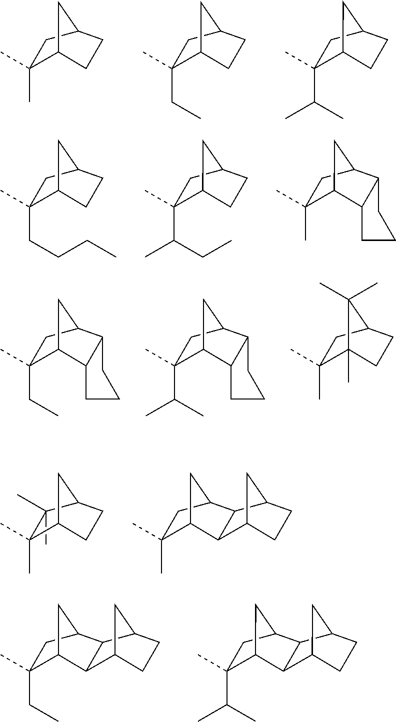

Examples of the tertiary C₄-C₂₀ alkyl groups, trialkylsilyl groups in which each alkyl moiety has 1 to 6 carbon atoms, and C₄-C₂₀ oxoalkyl groups, represented by X^{A}, are as exemplified for R^{L04}.

Illustrative examples of the recurring units of formula (a1) are given below, but not limited thereto. Herein R^{A} is as defined above.

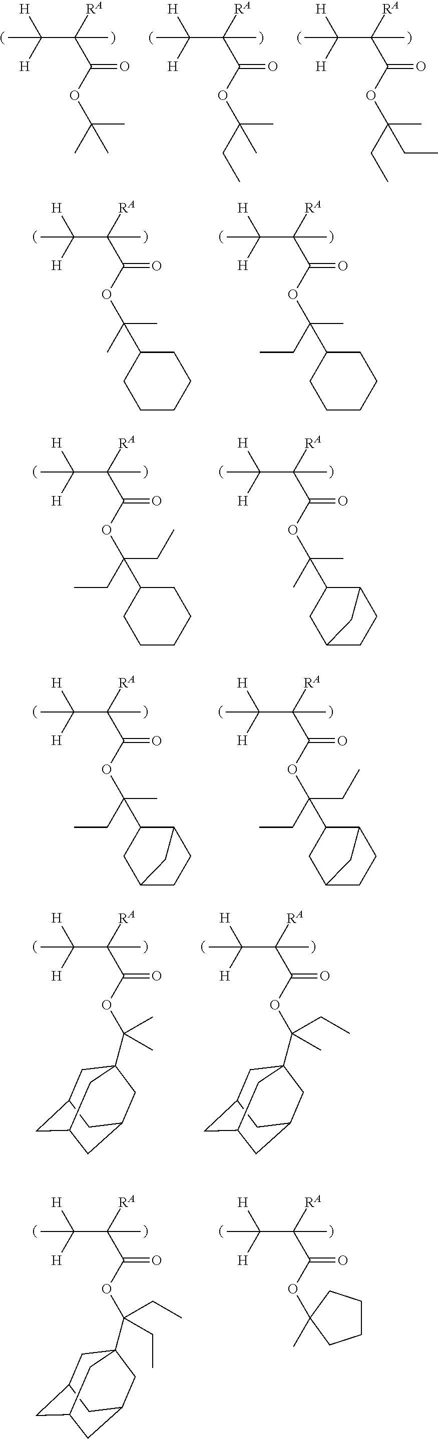

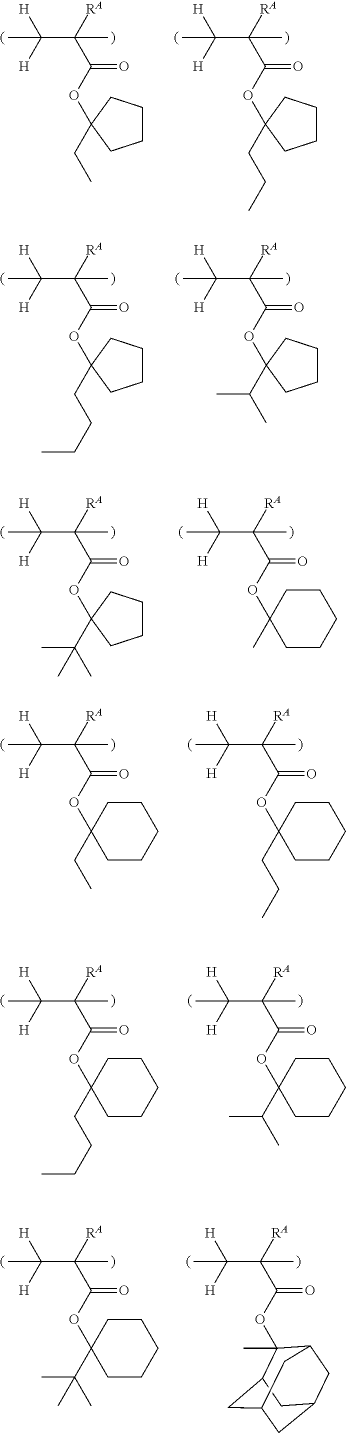

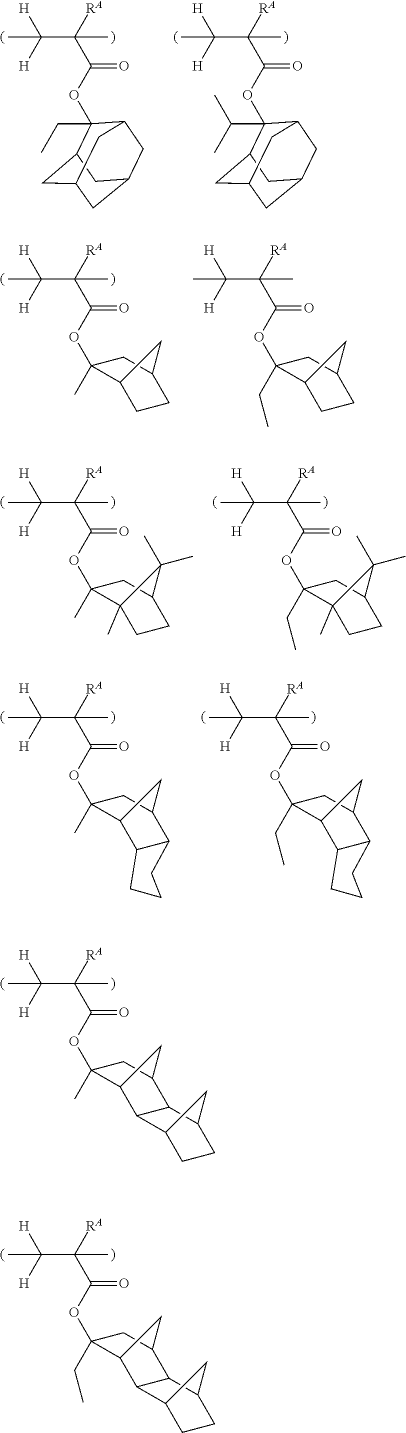

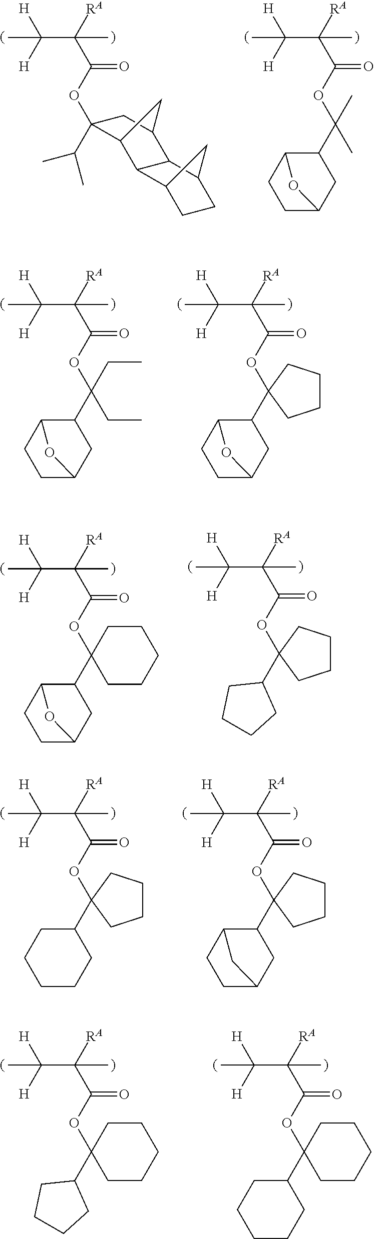

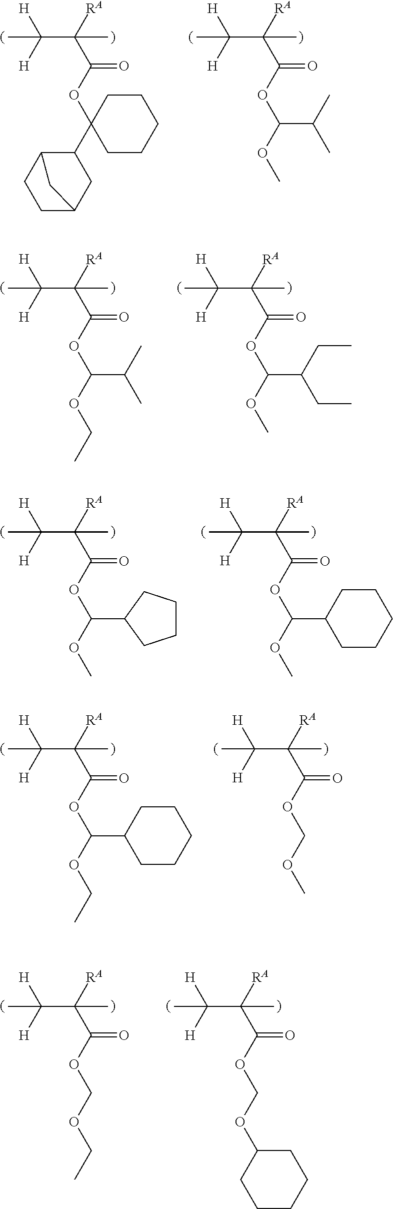

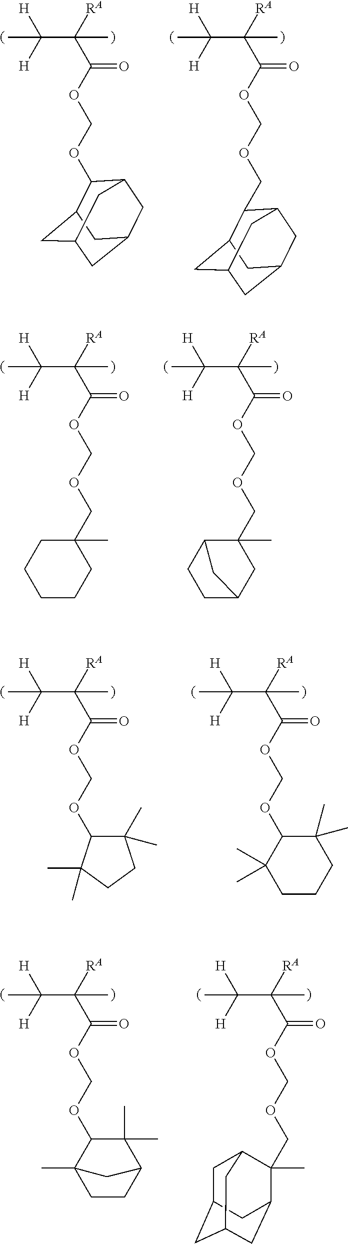

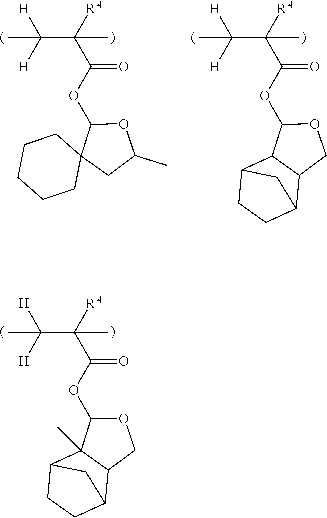

The above examples correspond to those units of formula (a1) wherein Z^{A} is a single bond. Where Z^{A} is other than a single bond, a combination with a similar acid labile group is possible. Thus examples of the recurring units of formula (a1) wherein Z^{A} is other than a single bond are as illustrated above.

In formula (a2), R^{B} is a C₁-C₂₀ straight, branched or cyclic monovalent hydrocarbon group which may contain a heteroatom. Examples of the monovalent hydrocarbon group are as exemplified above for R¹¹ to R¹⁹. The subscript n is an integer of 0 to 4, preferably 0 or 1.

Like a polymer comprising recurring units having formula (a1), a polymer comprising recurring units having formula (a2) is decomposed under the action of acid to generate a hydroxyl group and thus turns alkali soluble.

Illustrative examples of the recurring units of formula (a2) are given below, but not limited thereto. Herein R^{A} is as defined above.

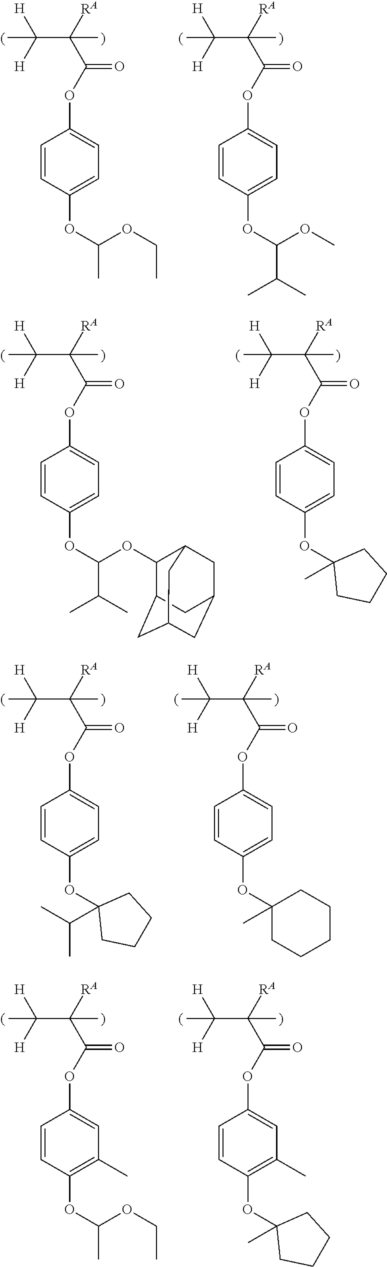

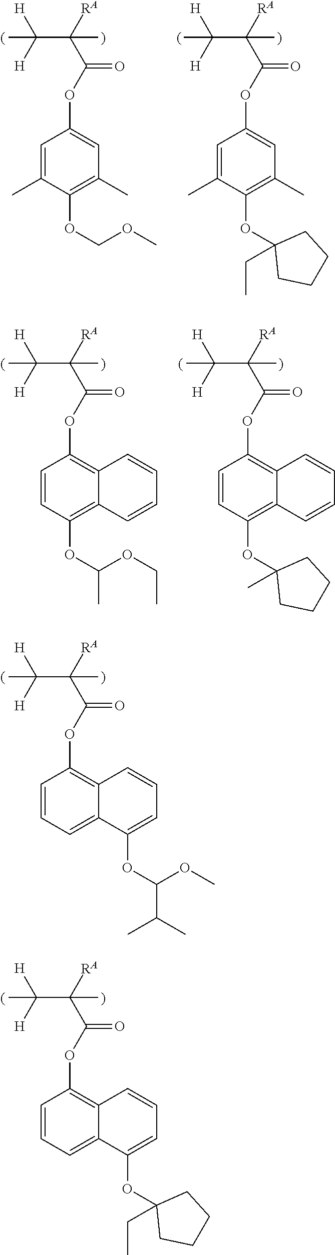

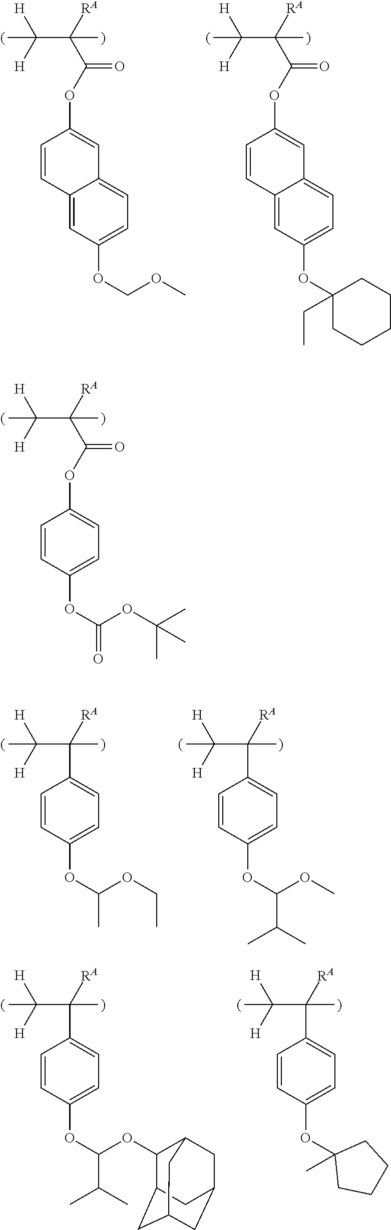

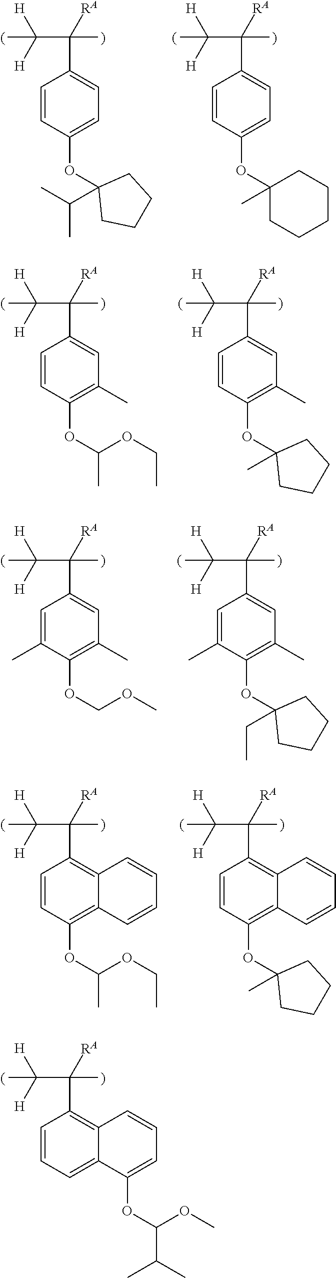

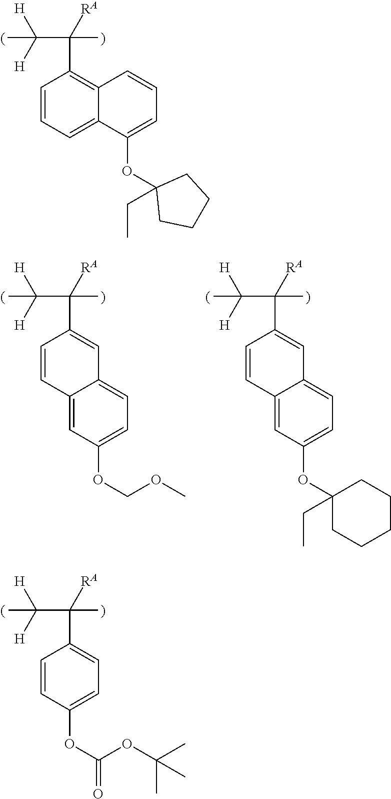

In a preferred embodiment, the polymer further comprises recurring units having the formula (b1) or (b2).

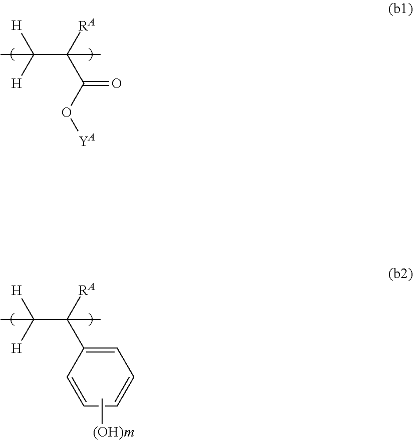

In formulae (b1) and (b2), R^{A} is as defined above. Y^{A} is hydrogen or a polar group containing at least one structure selected from among hydroxyl, cyano, carbonyl, carboxyl, ether bond, ester bond, sulfonic acid ester bond, carbonate bond, lactone ring, sultone ring and carboxylic anhydride, and m is 1 or 2.

Illustrative examples of the recurring units of formula (b1) are given below, but not limited thereto. Herein R^{A} is as defined above.

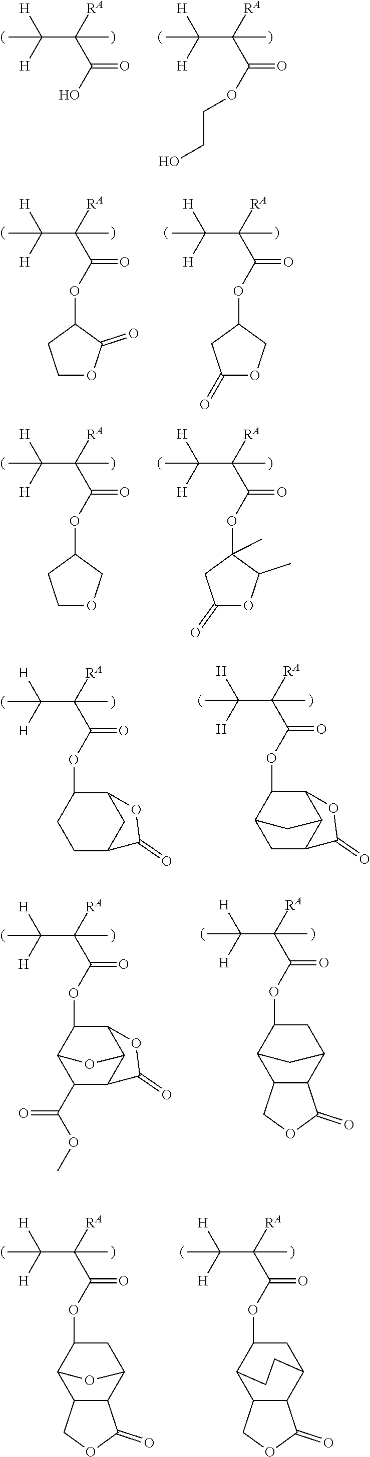

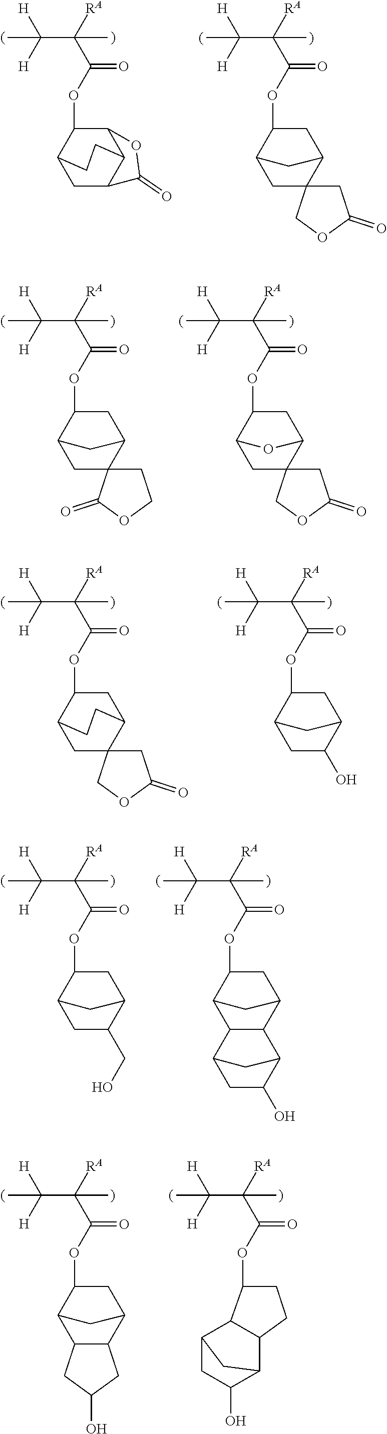

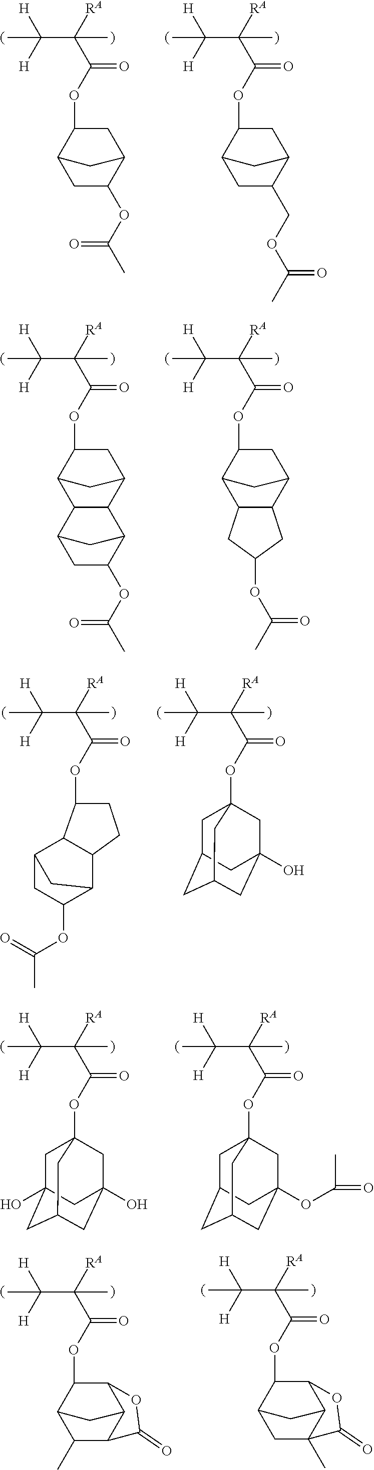

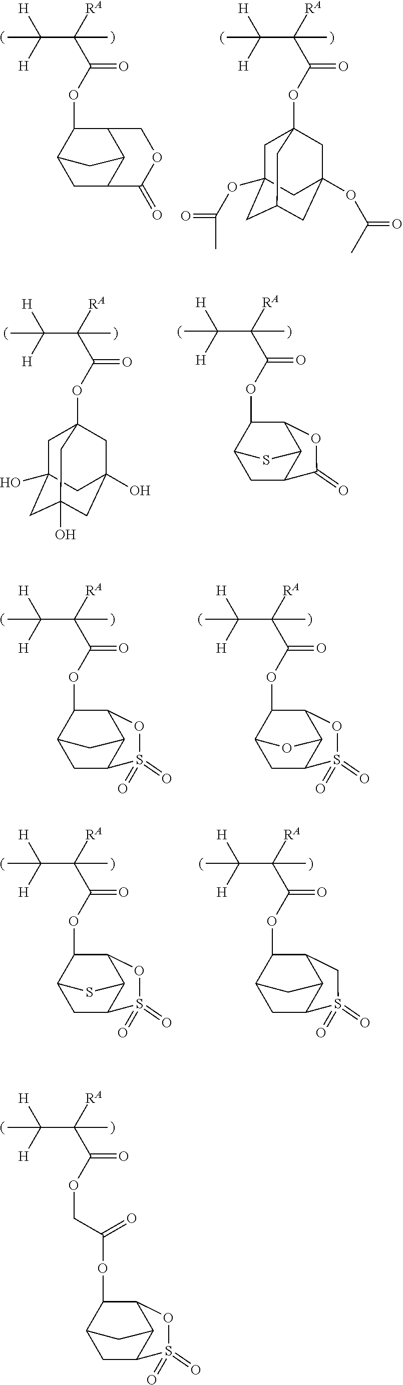

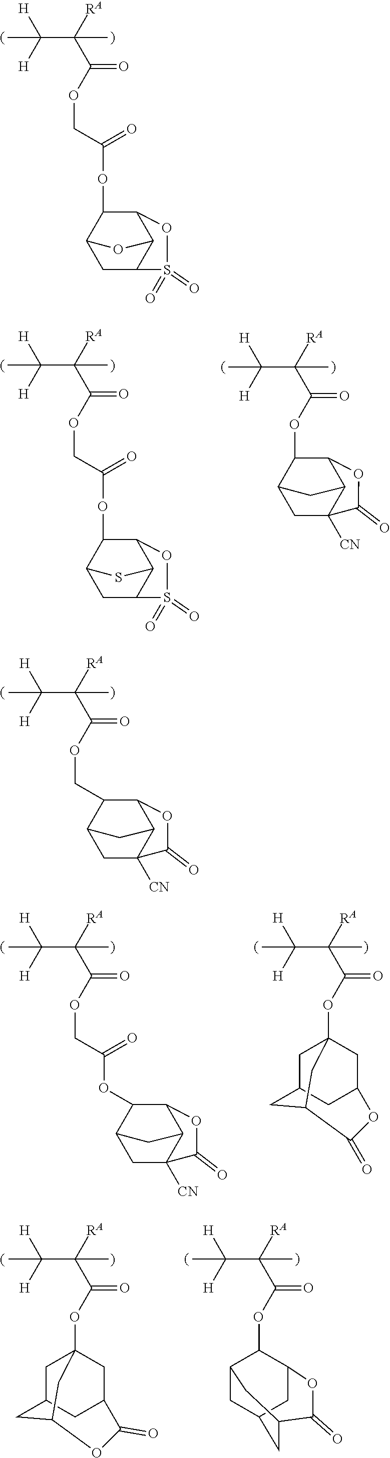

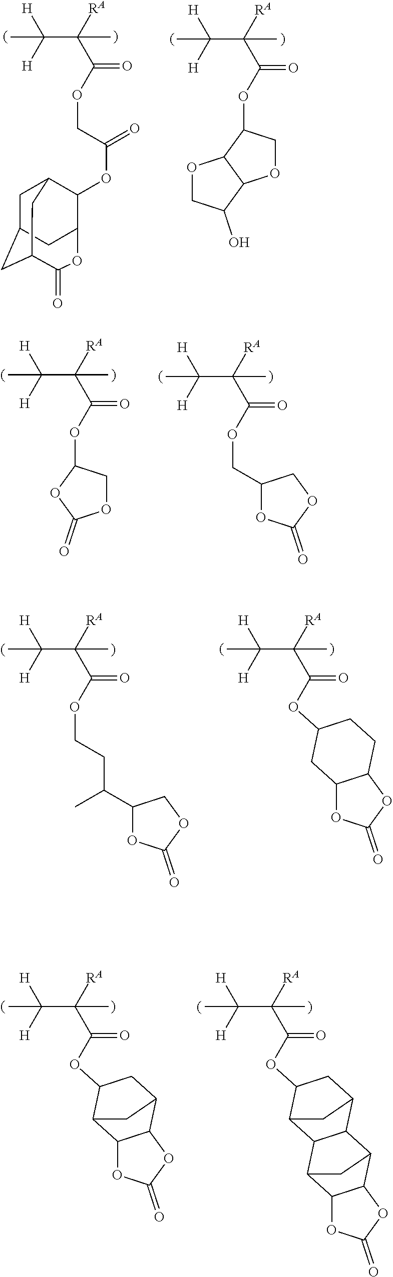

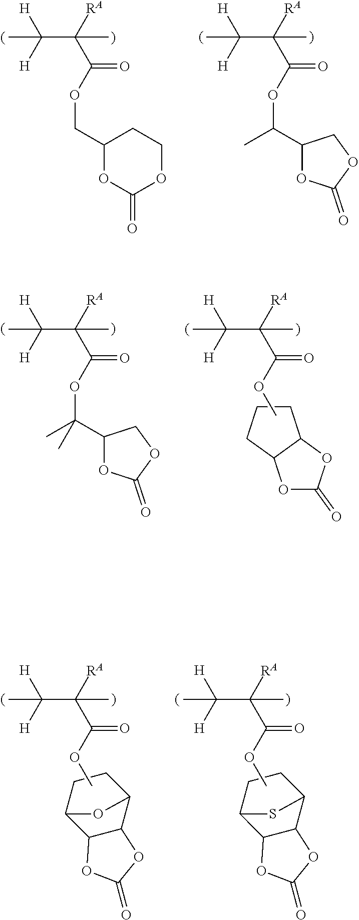

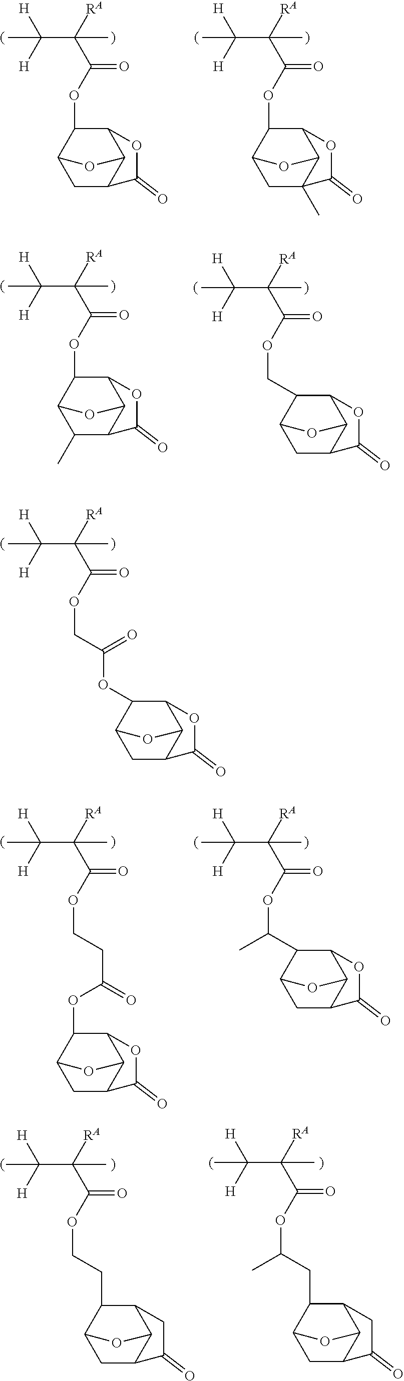

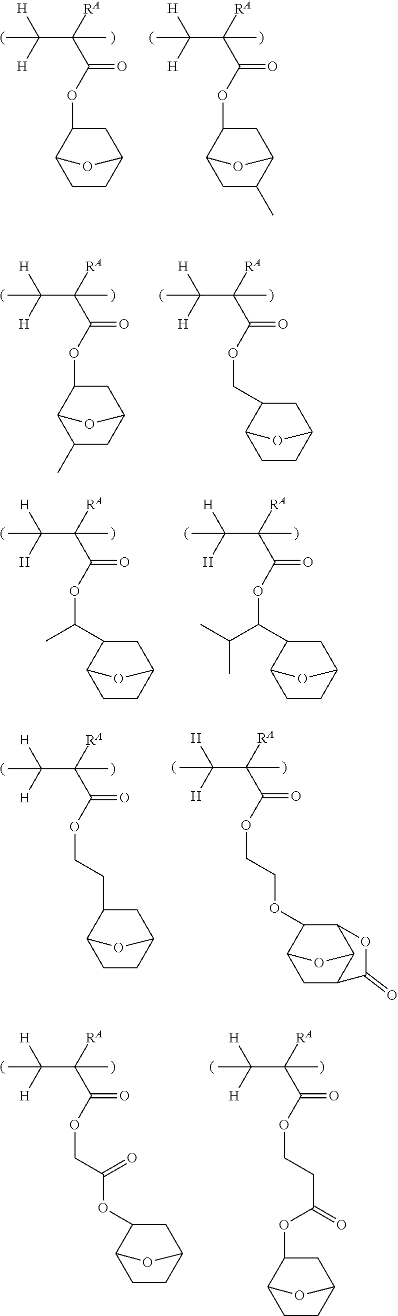

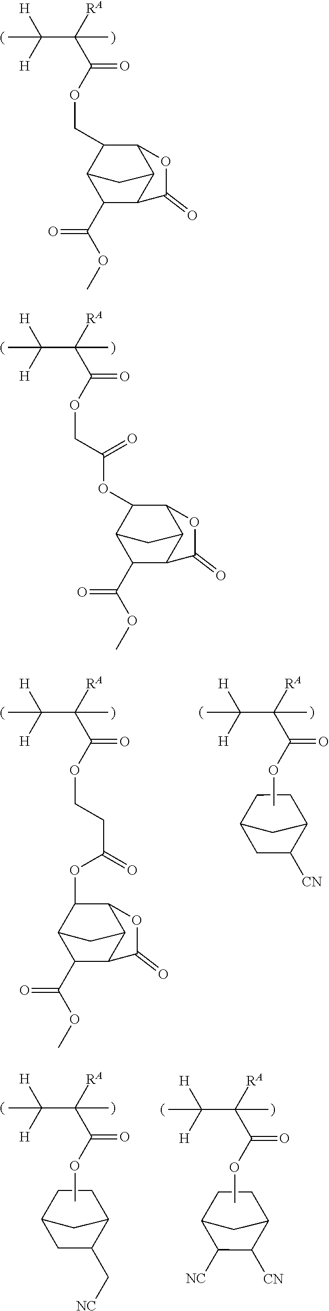

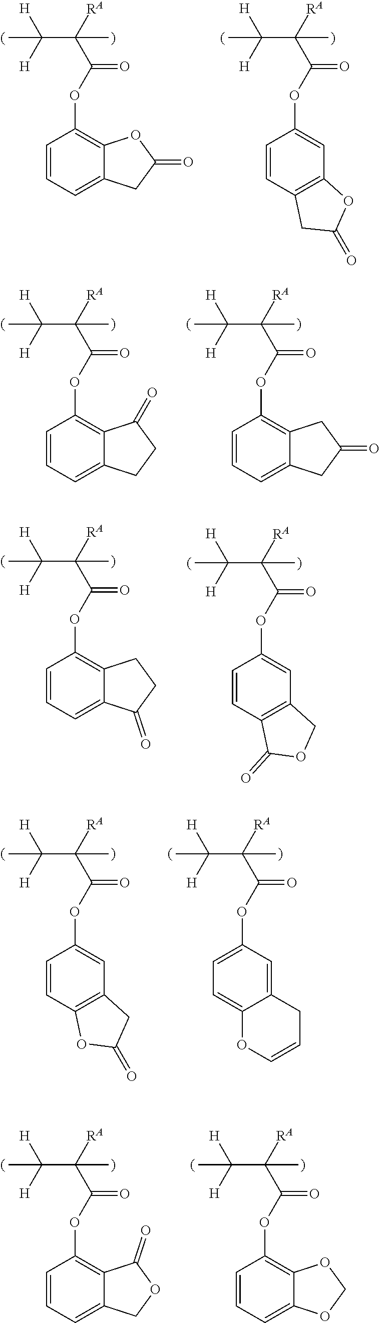

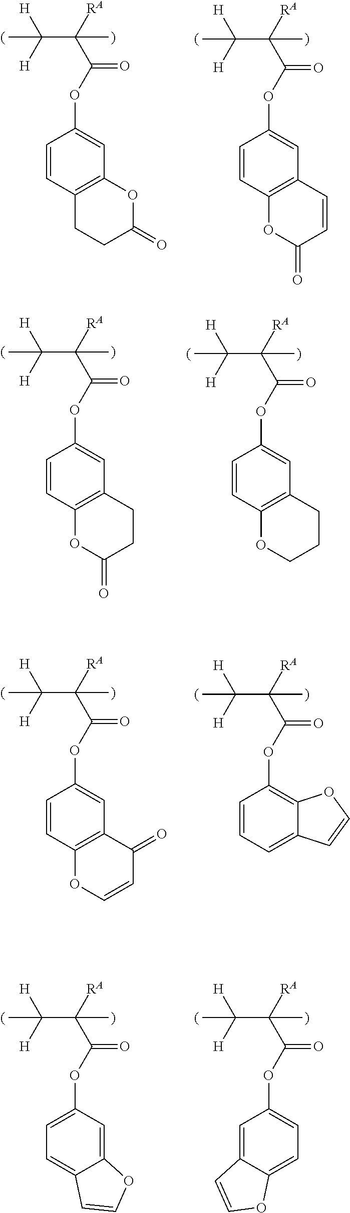

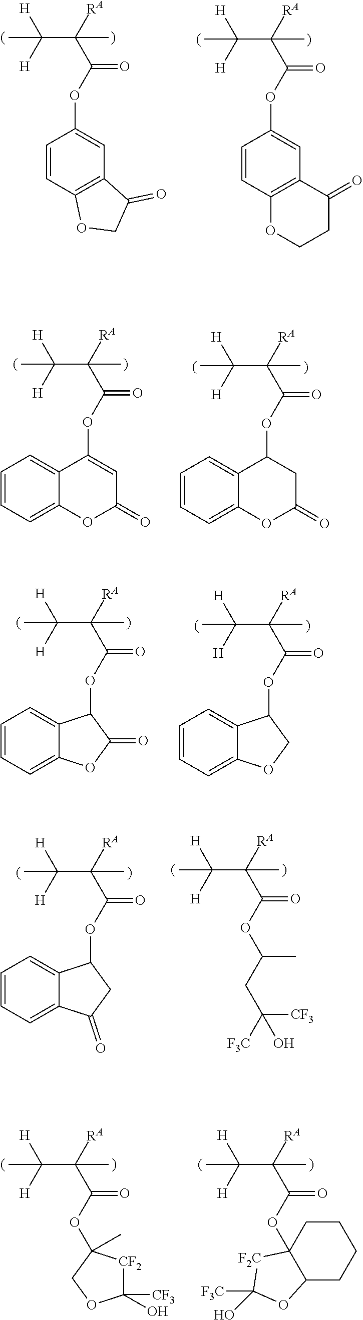

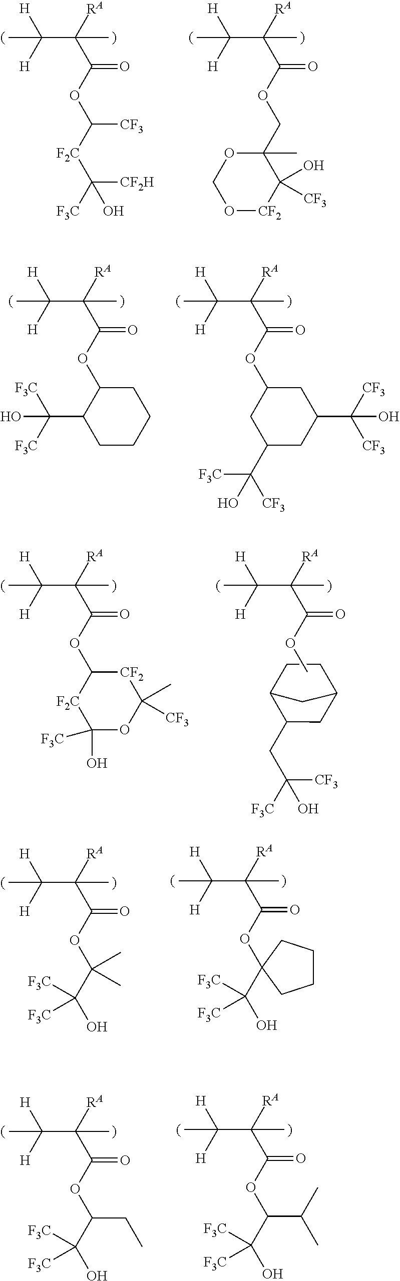

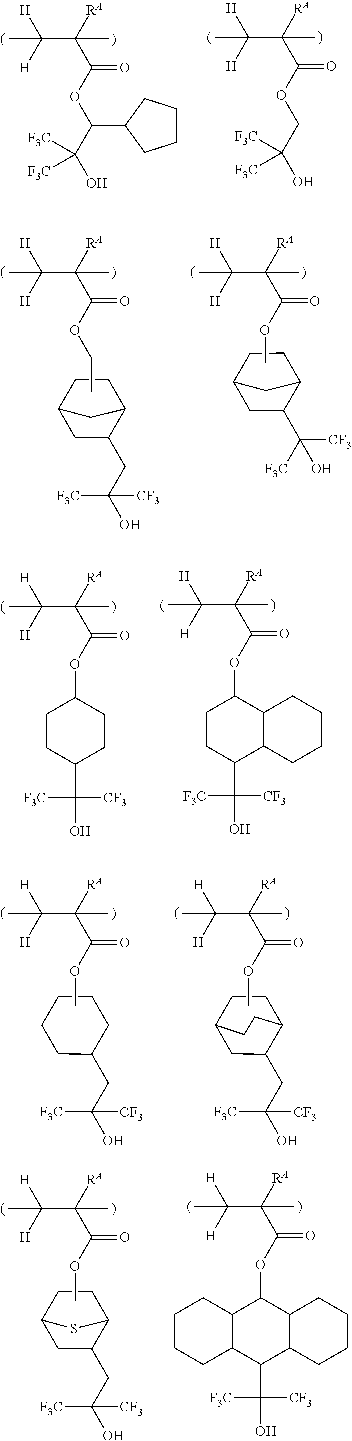

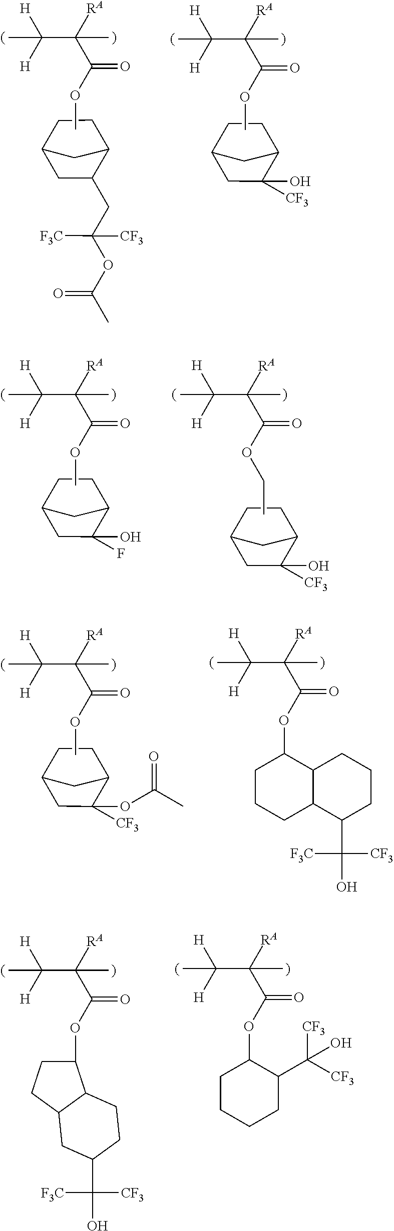

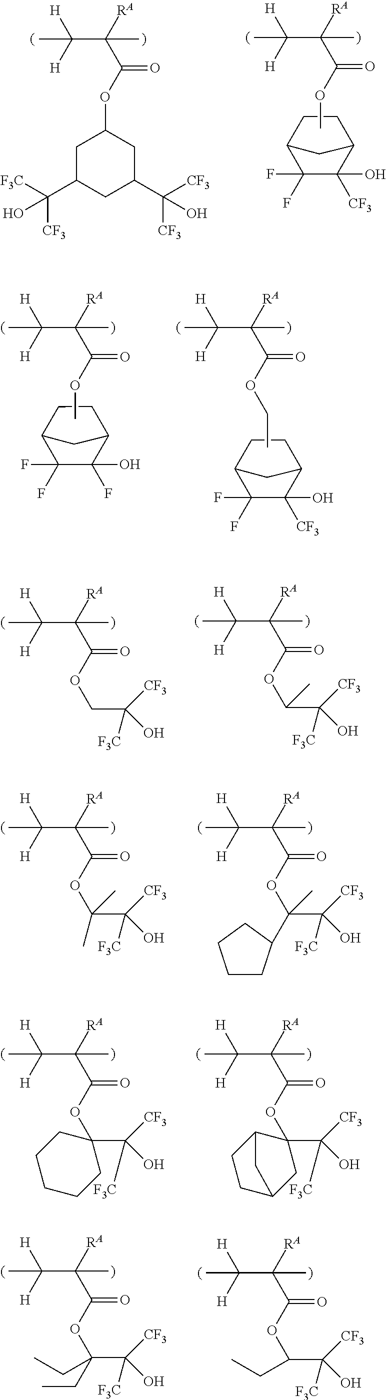

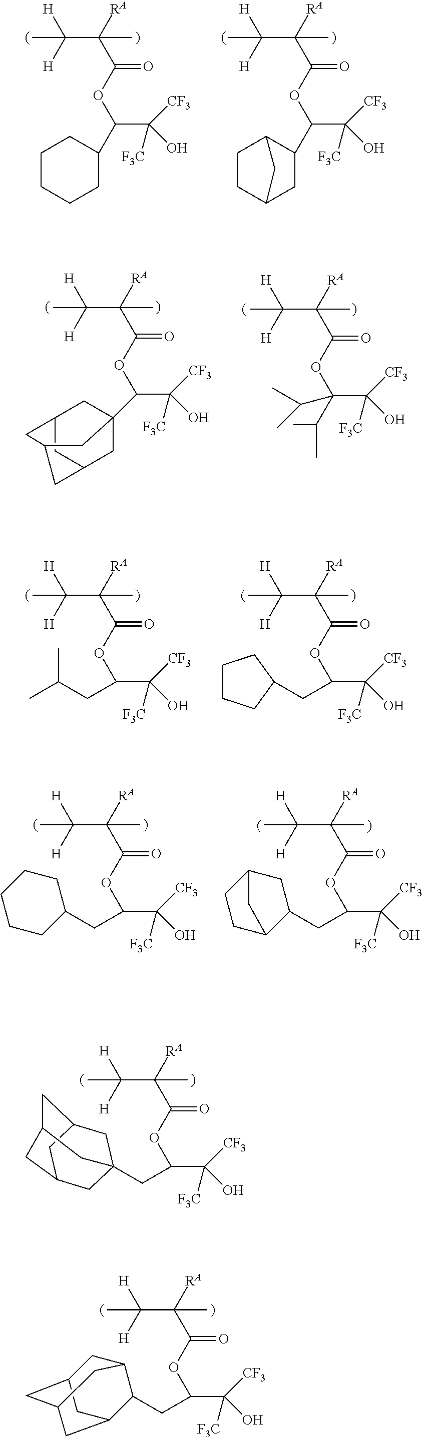

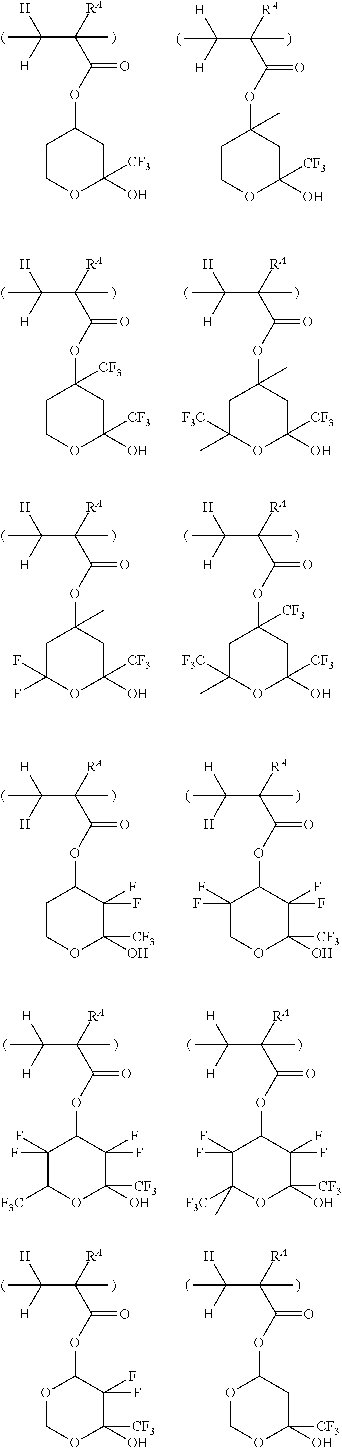

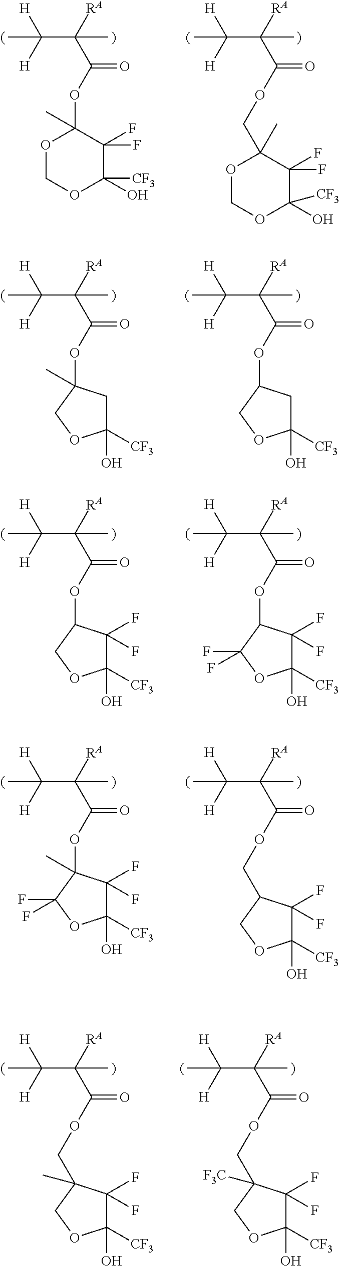

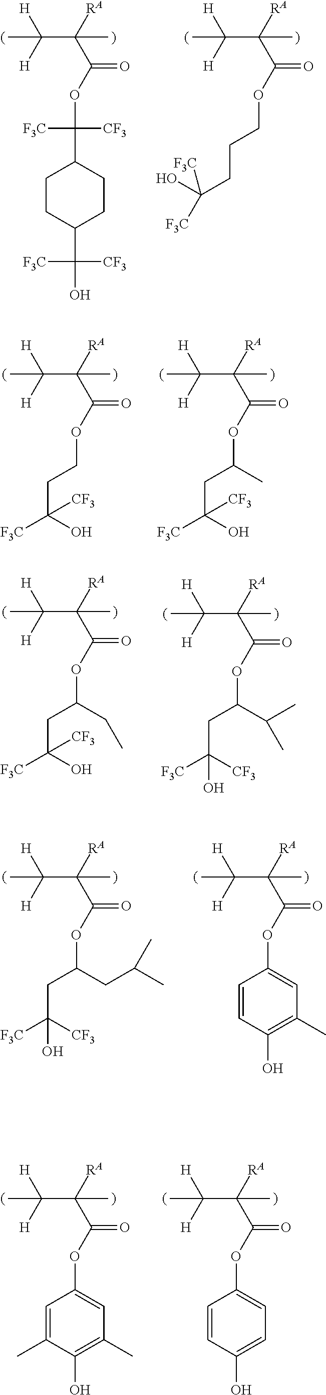

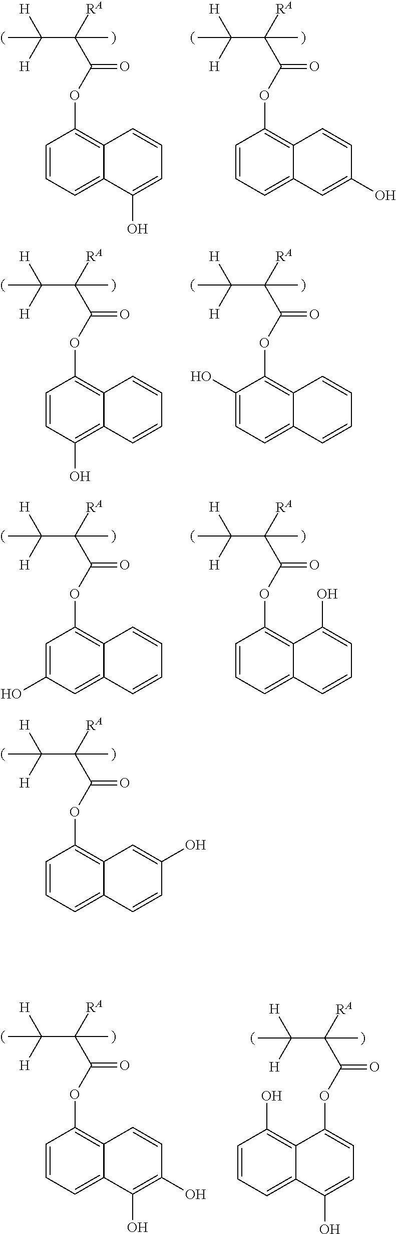

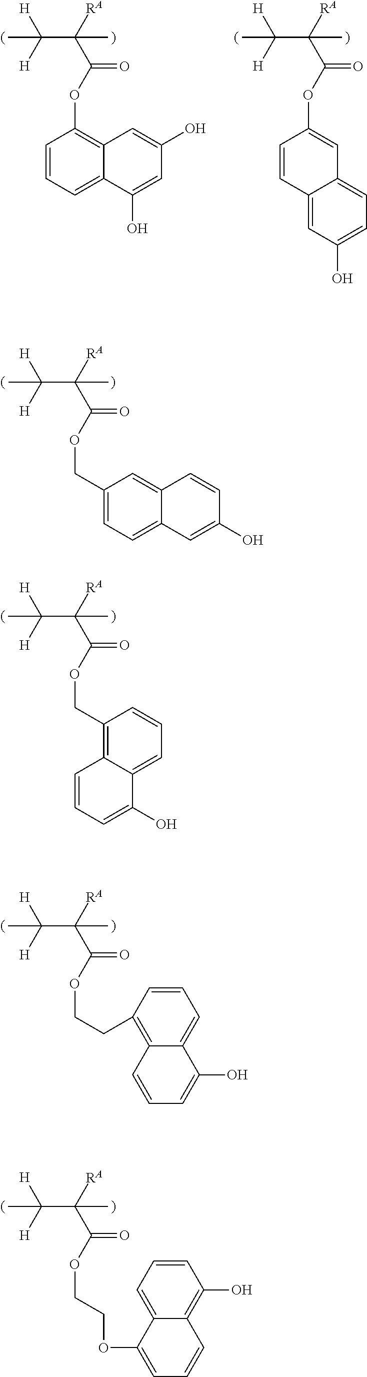

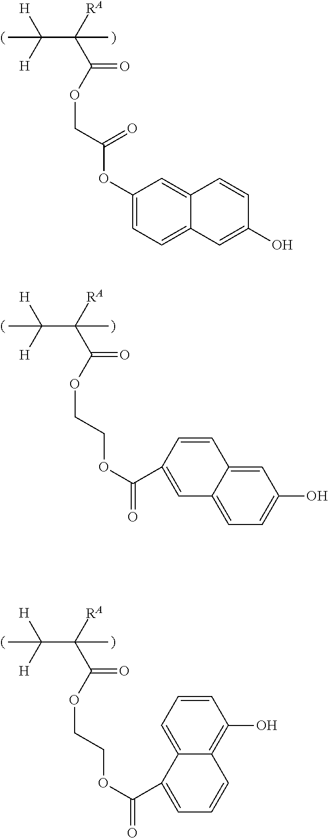

Illustrative examples of the recurring units of formula (b2) are given below, but not limited thereto. Herein R^{A} is as defined above.

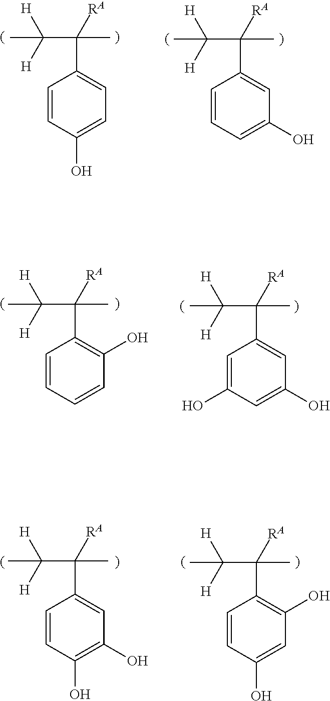

Of the recurring units of formula (b1) or (b2), those units having a lactone ring as the polar group are preferred in the ArF lithography process and those units having a phenol site are preferred in the KrF, EB and EUV lithography processes.

The polymer may further comprise recurring units having the formula (c1) or (c2).

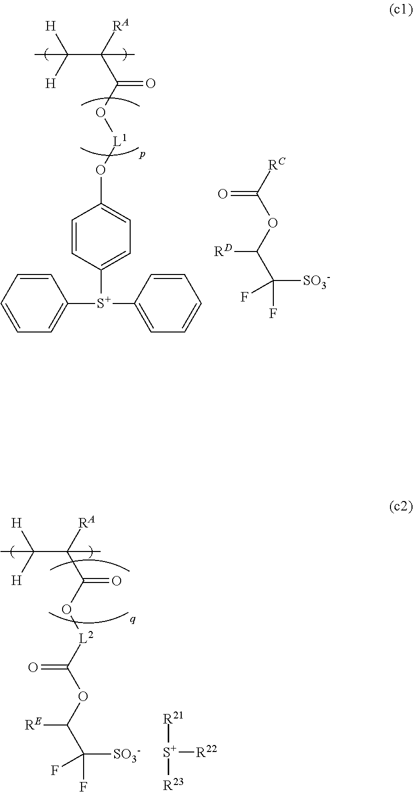

In formulae (c1) and (c2), R^{A} is as defined above. R²¹, R²² and R²³ are each independently a C₁-C₂₀ straight, branched or cyclic monovalent hydrocarbon group which may contain a heteroatom. L¹ is a C₂-C₅ alkanediyl group. R^{C} is a C₁-C₂₀ straight, branched or cyclic monovalent hydrocarbon group which may contain a heteroatom. R^{D} and R^{E} are each independently hydrogen or trifluoromethyl. L² is a single bond or a C₁-C₂₀ straight, branched or cyclic divalent hydrocarbon group which may contain a heteroatom, p is 0 or 1, and q is 0 or 1, q being 0 when L² is a single bond.

Examples of the monovalent hydrocarbon group represented by R^{C}, R²¹, R²² and R²³ are as exemplified above for R¹ to R³ in formula (1).

Examples of the alkanediyl group represented by L¹ include ethane-1,2-diyl, propane-1,2-diyl, propane-1,3-diyl, butane-1,3-diyl and butane-1,4-diyl.

R^{D} and R^{E} are preferably trifluoromethyl.

Examples of the divalent hydrocarbon group represented by L² include straight alkanediyl groups such as methylene, ethylene, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; divalent saturated cyclic hydrocarbon groups such as cyclopentanediyl, cyclohexanediyl, norbornanediyl and adamantanediyl; and arylene groups such as phenylene and naphthylene. In these groups, some hydrogen may be substituted by an alkyl moiety such as methyl, ethyl, propyl, n-butyl or tert-butyl, or a moiety containing a heteroatom such as oxygen, sulfur, nitrogen, or halogen, or some carbon may be replaced by a moiety containing a heteroatom such as oxygen, sulfur or nitrogen, so that the group may contain a hydroxyl moiety, cyano moiety, carbonyl moiety, ether bond, thioether bond, ester bond, sulfonate (sulfonic acid ester) bond, carbonate bond, carbamate bond, lactone ring, sultone ring, carboxylic anhydride or haloalkyl moiety.

Illustrative structures of the anion moiety in formula (c1) include those described in JP-A 2010-113209 and JP-A 2007-145797. Illustrative structures of the anion moiety in formula (c2) wherein R^{E} is hydrogen include those described in JP-A 2010-116550. Illustrative structures of the anion moiety in formula (c2) wherein R^{E} is trifluoromethyl include those described in JP-A 2010-077404.

In addition to the foregoing units, the polymer may further comprise other recurring units, for example, recurring units derived from monomers such as substituted acrylic esters such as methyl methacrylate, methyl crotonate, dimethyl maleate and dimethyl itaconate, unsaturated carboxylic acids such as maleic acid, fumaric acid, and itaconic acid, cyclic olefins such as norbornene, norbornene derivatives, and tetracyclo[6.2.1.1^{3,6}.0^{2,7}]dodecene derivatives, unsaturated acid anhydrides such as itaconic anhydride, and other monomers.

The polymer generally has a weight average molecular weight (Mw) of 1,000 to 500,000, and preferably 3,000 to 100,000, as measured versus polystyrene standards by gel permeation chromatography (GPC) using tetrahydrofuran (THF) solvent. When Mw is within the range, satisfactory etch resistance is achievable, and a drop of resolution due to difficulty to gain a dissolution rate difference before and after exposure may be avoided.

If a polymer has a wide molecular weight distribution or dispersity (Mw/Mn), which indicates the presence of lower and higher molecular weight polymer fractions, there is a possibility that foreign matter is left on the pattern or the pattern profile is degraded. The influences of molecular weight and dispersity become stronger as the pattern rule becomes finer. Therefore, the polymer should preferably have a narrow dispersity (Mw/Mn) of 1.0 to 2.0 in order to provide a resist composition suitable for micropatterning to a small feature size.

A single polymer or a blend of two or more polymers which differ in compositional ratio, Mw and/or Mw/Mn may be used as the base resin (C).

The polymer may be synthesized by any desired method, for example, by dissolving an unsaturated bond-containing monomer or monomers in an organic solvent, adding a radical initiator, and heating for polymerization. Suitable organic solvents used herein include toluene, benzene, tetrahydrofuran, diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably the reaction temperature is 50 to 80° C. and the reaction time is 2 to 100 hours, more preferably 5 to 20 hours. The acid labile group that has been incorporated in the monomer may be kept as such, or the polymer may be protected or partially protected therewith at the end of polymerization.

In the polymer, appropriate molar fractions (mol %) of the respective recurring units derived from the monomers are given below although the invention is not limited thereto.

The polymer may comprise:

(I) 1 to 60 mol %, preferably 5 to 50 mol %, and more preferably 10 to 50 mol % of recurring units of at least one type having formula (a1) or (a2),

(II) 40 to 99 mol %, preferably 50 to 95 mol %, and more preferably 50 to 90 mol % of recurring units of at least one type having formula (b1) or (b2), and

(III) 0 to 50 mol %, preferably 0 to 40 mol %, and more preferably 0 to 30 mol % of recurring units of at least one type derived from another monomer.

### (D) Photoacid Generator

The resist composition may further comprise a photoacid generator (PAG). The PAG may be any compound capable of generating an acid upon exposure to high-energy radiation such as UV, deep UV, EB, EUV, x-ray, excimer laser, γ-ray, and synchrotron radiation. Suitable PAGs include sulfonium salts, iodonium salts, sulfonyldiazomethanes, N-sulfonyloxydicarboxyimides, O-arylsulfonyloximes, and O-alkylsulfonyloximes, which may be used alone or in admixture. Suitable examples are described in JP-A 2007-145797, paragraphs [0102]-[0113], for example.

Salts having the formula (2) are preferred as the PAG (D).

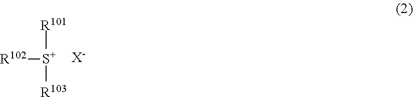

In formula (2), R¹⁰¹, R¹⁰² and R¹⁰³ are each independently a C₁-C₂₀ straight, branched or cyclic monovalent hydrocarbon group which may contain a heteroatom. Any two of R¹⁰¹, R¹⁰² and R¹⁰³ may bond together to form a ring with the sulfur atom to which they are attached. The monovalent hydrocarbon group is as defined and exemplified above for R¹¹ to R¹⁹. Examples of the sulfonium cation in formula (2) are as exemplified above for the sulfonium cation having formula (1-1).

In formula (2), X⁻ is an anion selected from the formulae (2A) to (2D).

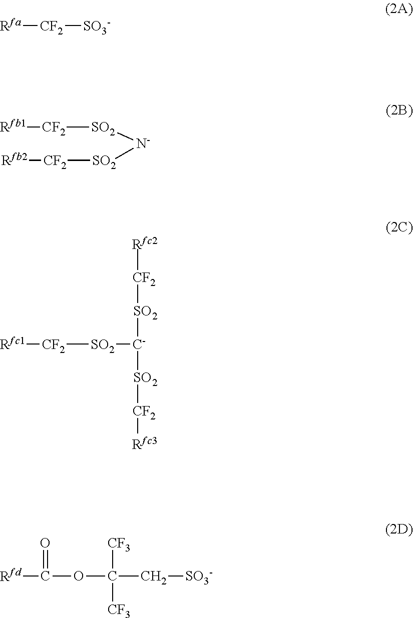

In formula (2A), R^{fa} is fluorine or a C₁-C₄₀ straight, branched or cyclic monovalent hydrocarbon group which may contain a heteroatom. The monovalent hydrocarbon group is the same as will be defined and exemplified below for R¹¹².

Of the anions of formula (2A), a structure having the formula (2A′) is especially preferred.

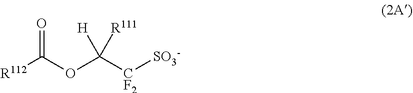

In formula (2A′), R¹¹¹ is hydrogen or trifluoromethyl. R¹¹² is a C₁-C₃₀ straight, branched or cyclic monovalent hydrocarbon group which may contain a heteroatom. Suitable heteroatoms include oxygen, nitrogen, sulfur and halogen, with oxygen being preferred. Of the monovalent hydrocarbon groups, those of 6 to 30 carbon atoms are preferred because a high resolution is available in fine pattern formation.

Examples of the monovalent hydrocarbon group include straight or branched alkyl groups such as methyl, ethyl, propyl, isopropyl, butyl, isobutyl, sec-butyl, tert-butyl, pentyl, neopentyl, hexyl, heptyl, 2-ethylhexyl, nonyl, undecyl, tridecyl, pentadecyl, heptadecyl, and icosanyl; monovalent saturated cycloaliphatic hydrocarbon groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, dicyclohexylmethyl; monovalent unsaturated aliphatic hydrocarbon groups such as allyl and 3-cyclohexenyl; aryl groups such as phenyl, 1-naphthyl and 2-naphthyl; and aralkyl groups such as benzyl and diphenylmethyl. Examples of the heteroatom-containing monovalent hydrocarbon group include tetrahydrofuryl, methoxymethyl, ethoxymethyl, methylthiomethyl, acetamidomethyl, trifluoromethyl, (2-methoxyethoxy)methyl, acetoxymethyl, 2-carboxy-1-cyclohexyl, 2-oxopropyl, 4-oxo-1-adamantyl, and 3-oxocyclohexyl. In these groups, some hydrogen may be substituted by a moiety containing a heteroatom such as oxygen, sulfur, nitrogen or halogen, or some carbon may be replaced by a moiety containing a heteroatom such as oxygen, sulfur or nitrogen, so that the group may contain a hydroxyl, cyano, carbonyl, ether, ester, sulfonic acid ester, carbonate, lactone ring, sultone ring, carboxylic anhydride or haloalkyl moiety.

With respect to the synthesis of a sulfonium salt having the anion of formula (2A′), reference is made to JP-A 2007-145797, JP-A 2008-106045, JP-A 2009-007327, and JP-A 2009-258695.

Examples of the anion having formula (2A) include nonafluorobutane sulfonate ions, partially fluorinated sulfonate ions described in JP-A 2012-189977, paragraphs [0247]-[0251], and partially fluorinated sulfonate ions described in JP-A 2013-101271, paragraphs [0261]-[0265].

Further examples of the anion having formula (2A) include the following, but are not limited thereto.

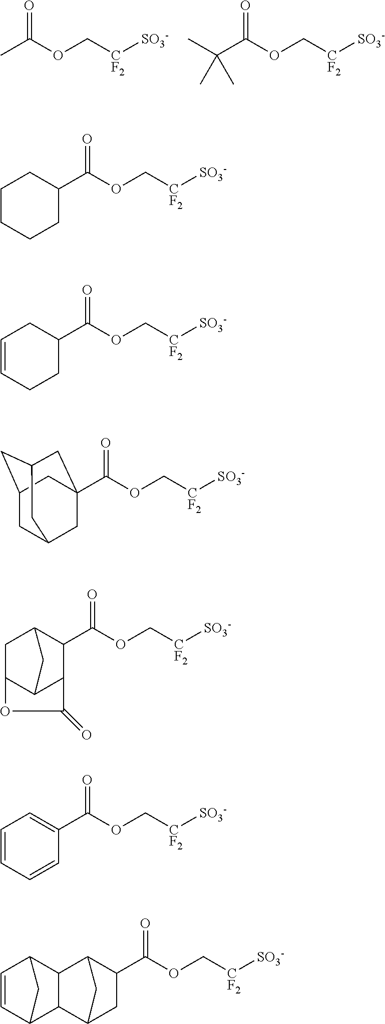

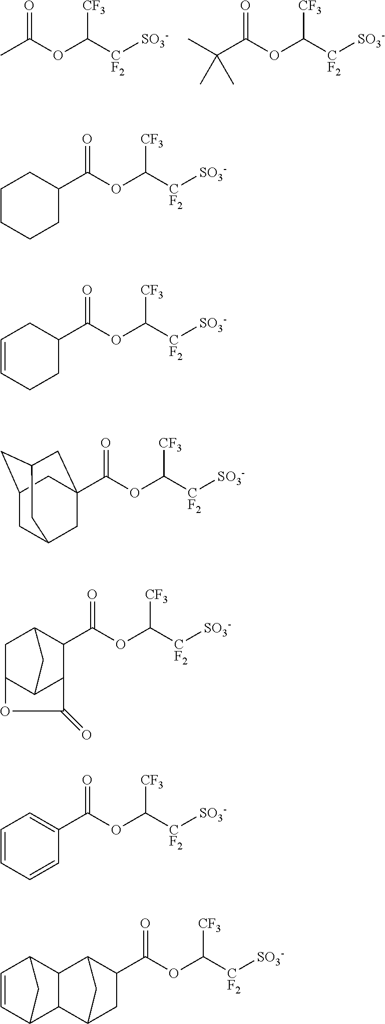

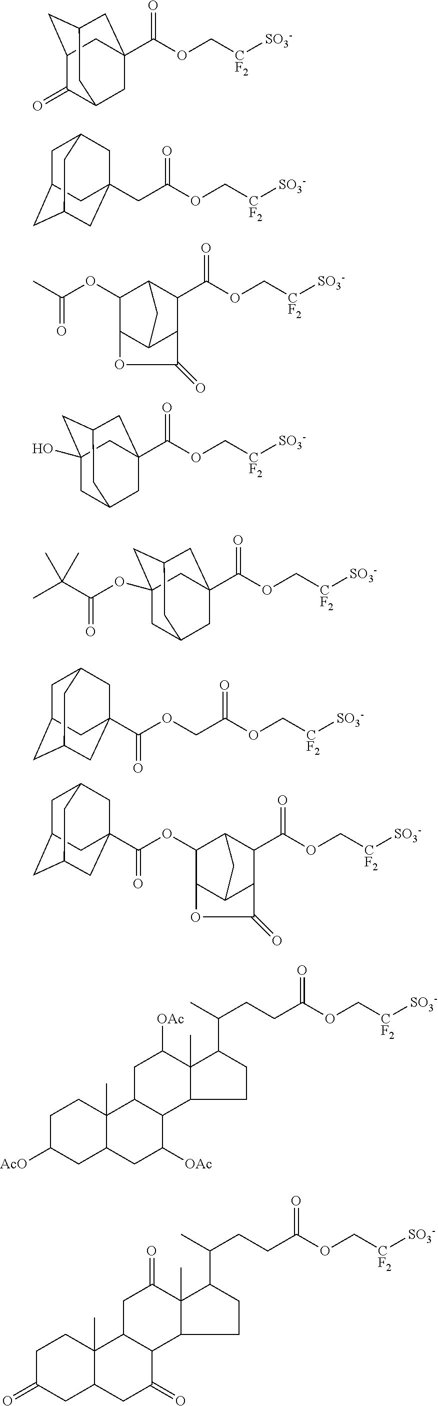

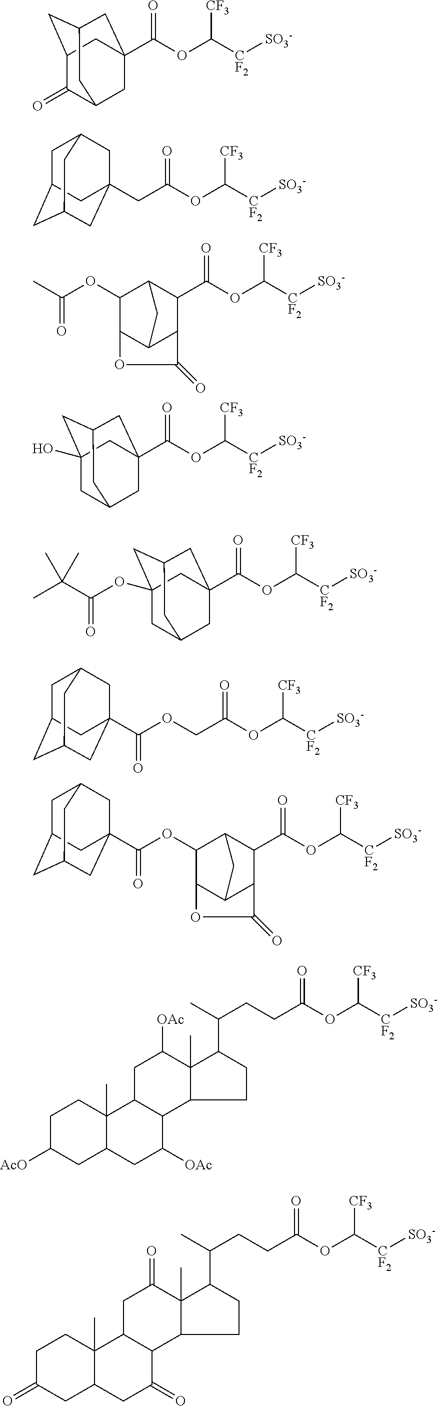

In formula (2B), R^{fb1} and R^{fb2} are each independently fluorine or a C₁-C₄₀ straight, branched or cyclic monovalent hydrocarbon group which may contain a heteroatom. Examples of the monovalent hydrocarbon group are as exemplified for R¹¹². Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. In this case, the combination of R^{fb1} and R^{fb2} is preferably a fluorinated ethylene or fluorinated propylene group.

In formula (2C), R^{fc1}, R^{fc2} and R^{fc3} are each independently fluorine or a C₁-C₄₀ straight, branched or cyclic monovalent hydrocarbon group which may contain a heteroatom. Examples of the monovalent hydrocarbon group are as exemplified for R¹¹². Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C—SO₂—CF₂— to which they are attached. The combination of R^{fc1} and R^{fc2} is preferably a fluorinated ethylene or fluorinated propylene group.

In formula (2D), R^{fd} is a C₁-C₄₀ straight, branched or cyclic monovalent hydrocarbon group which may contain a heteroatom. Examples of the monovalent hydrocarbon group are as exemplified above for R¹¹².

With respect to the synthesis of a sulfonium salt having the anion of formula (2D), reference is made to JP-A 2010-215608.

Examples of the anion having formula (2D) include the following, but are not limited thereto.

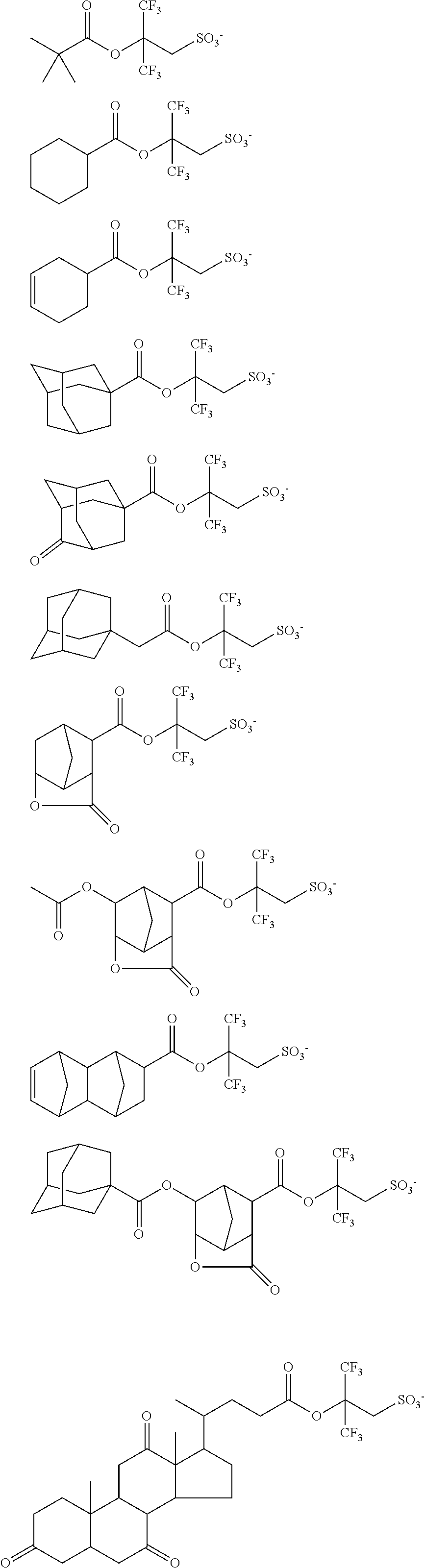

Notably, the compound having the anion of formula (2D) does not have fluorine at the α-position relative to the sulfo group, but two trifluoromethyl groups at the β-position. For this reason, it has a sufficient acidity to sever the acid labile groups in the resist polymer. Thus the compound is an effective PAG.

Another preferred PAG (D) is a compound having the formula (3).

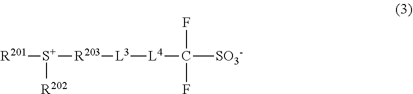

In formula (3), R²⁰¹ and R²⁰² are each independently a C₁-C₂₀ straight, branched or cyclic monovalent hydrocarbon group which may contain a heteroatom. R²⁰³ is a C₁-C₂₀ straight, branched or cyclic divalent hydrocarbon group which may contain a heteroatom. Any two of R²⁰¹, R²⁰² and R²⁰³ may bond together to form a ring with the sulfur atom to which they are attached. L³ is a divalent linking group. L^{A} is a single bond or a C₁-C₂₀ straight, branched or cyclic divalent hydrocarbon group which may contain a heteroatom.

Examples of the monovalent hydrocarbon groups represented by R²⁰¹ and R²⁰² include methyl, ethyl, propyl, isopropyl, n-butyl, sec-butyl, tert-butyl, n-pentyl, tert-pentyl, n-hexyl, n-octyl, cyclopentyl, cyclohexyl, 2-ethylhexyl, cyclopentylmethyl, cyclopentylethyl, cyclohexylmethyl, cyclohexylethyl, norbornyl, oxanorbornyl, tricyclo[5.2.1.0^{2,6}]decanyl, adamantyl, phenyl, and naphthyl. In these groups, some hydrogen may be substituted by a moiety containing a heteroatom such as oxygen, sulfur, nitrogen, or halogen, or a moiety containing a heteroatom such as oxygen, sulfur or nitrogen may intervene between carbon atoms, so that the group may contain a hydroxyl moiety, cyano moiety, carbonyl moiety, ether bond, ester bond, sulfonate bond, carbonate bond, lactone ring, sultone ring, carboxylic anhydride or haloalkyl moiety. Of these, optionally substituted aryl groups are preferred as R²⁰¹ and R²⁰².

Examples of the divalent hydrocarbon group represented by R²⁰³ include straight alkanediyl groups such as methylene, ethylene, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; saturated cyclic hydrocarbon groups such as cyclopentanediyl, cyclohexanediyl, norbornanediyl and adamantanediyl; and unsaturated cyclic hydrocarbon groups such as phenylene and naphthylene. In these groups, some hydrogen may be substituted by an alkyl moiety such as methyl, ethyl, propyl, n-butyl or tert-butyl, or a moiety containing a heteroatom such as oxygen, sulfur, nitrogen, or halogen; some hydrogen may be substituted by a moiety containing a heteroatom such as oxygen, sulfur or nitrogen may intervene between carbon atoms, so that the group may contain a hydroxyl moiety, cyano moiety, carbonyl moiety, ether bond, ester bond, sulfonate bond, carbonate bond, lactone ring, sultone ring, carboxylic anhydride or haloalkyl moiety. Of these, optionally substituted aryl groups are preferred as R²⁰³.

Examples of the linking group represented by L³ include an ether bond, ester bond, thioether bond, sulfinic acid ester bond, sulfonic acid ester bond, carbonate bond, and carbamate bond.

Examples of the divalent hydrocarbon group represented by L⁴ include straight alkanediyl groups such as methylene, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; saturated cyclic hydrocarbon groups such as cyclopentanediyl, cyclohexanediyl, norbornanediyl and adamantanediyl; and unsaturated cyclic hydrocarbon groups such as phenylene and naphthylene. In these groups, some hydrogen may be substituted by an alkyl moiety such as methyl, ethyl, propyl, n-butyl or tert-butyl; some hydrogen may be substituted by a moiety containing a heteroatom such as oxygen, sulfur, nitrogen, or halogen, or a moiety containing a heteroatom such as oxygen, sulfur or nitrogen may intervene between carbon atoms, so that the group may contain a hydroxyl moiety, cyano moiety, carbonyl moiety, ether bond, ester bond, sulfonate bond, carbonate bond, lactone ring, sultone ring, carboxylic anhydride or haloalkyl moiety. Of these, methylene, and methylene groups substituted with fluorine or trifluoromethyl are preferred as L⁴.

Examples of the PAG having formula (3) are shown below, but not limited thereto. Herein R is hydrogen, fluorine or trifluoromethyl.

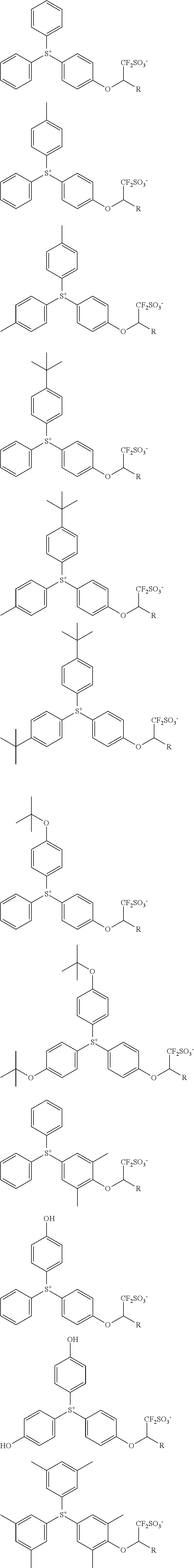

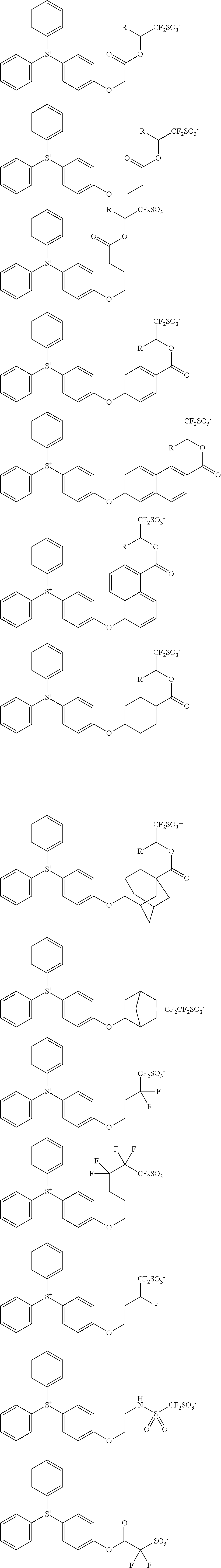

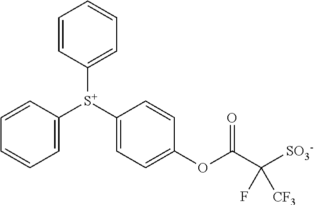

The amount of PAG (D) used is preferably 0 to 40 parts by weight, and when added, 0.1 to 40 parts by weight, more preferably 0.1 to 20 parts by weight per 100 parts by weight of the base resin (C). As long as the amount of PAG is in the range, a satisfactory resolution is achieved and the problem of foreign particles after resist development or during stripping is avoided. The PAG may be used alone or in admixture.

### (E) Surfactant

The resist composition may further comprise (E) a surfactant which is commonly used for facilitating coating operation. For the surfactant, reference should be made to those compounds described in JP-A 2010-215608 and JP-A 2011-016746.

Component (E) is typically a surfactant which is insoluble or substantially insoluble in water and soluble in alkaline developer, or a surfactant (hydrophobic resin) which is insoluble or substantially insoluble in water and alkaline developer.

While many examples of the surfactant which is insoluble or substantially insoluble in water and alkaline developer are described in the patent documents cited herein, preferred to examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

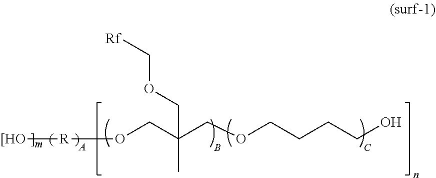

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

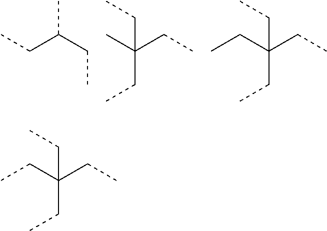

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, “B” is an integer of 2 to 25, and “C” is an integer of 0 to 10. Preferably, “B” is an integer of 4 to 20, and “C” is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface after spin coating for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing recurring units of at least one type selected from the formulae (4) to (8).

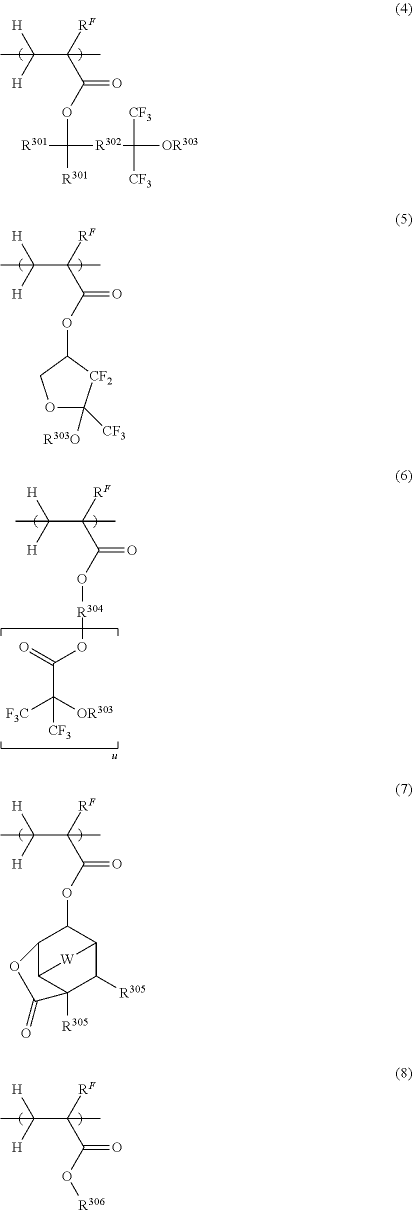

In formulae (4) to (8), R^{F} is hydrogen or methyl. W is —CH₂—, —CH₂CH₂— or —O—, or two separate —H. R³⁰¹ is each independently hydrogen or a C₁-C₁₀ straight, branched or cyclic monovalent hydrocarbon group. R³⁰² is a single bond or a C₁-C₅ straight or branched divalent hydrocarbon group. R³⁰³ is each independently hydrogen, a C₁-C₁₅ straight, branched or cyclic monovalent hydrocarbon or fluorinated hydrocarbon group, or an acid labile group. When R³⁰³ is a monovalent hydrocarbon or fluorinated hydrocarbon group, an ether bond (—O—) or carbonyl moiety (—C(═O)—) may intervene in a carbon-carbon bond. R³⁰⁴ is a (u+1)-valent straight, branched or cyclic hydrocarbon or fluorinated hydrocarbon group, and u is an integer of 1 to 3. R³⁰⁵ is each independently hydrogen or a group having the formula (i):

—C(═O)—O—R^{305A} (i)

wherein R^{305A} is a C₁-C₂₀ straight, branched or cyclic fluorinated hydrocarbon group. R³⁰⁶ is a C₁-C₁₅ straight, branched or cyclic monovalent hydrocarbon or fluorinated hydrocarbon group in which an ether bond (—O—) or carbonyl moiety (—C(═O)—) may intervene in a carbon-carbon bond.

Examples of the monovalent hydrocarbon group represented by R³⁰¹ include methyl, ethyl, n-propyl, isopropyl, cyclopropyl, n-butyl, isobutyl, sec-butyl, tert-butyl, cyclobutyl, n-pentyl, cyclopentyl, n-hexyl, cyclohexyl, n-heptyl, n-octyl, n-nonyl, n-decyl, adamantyl, and norbornyl. Inter alia, C₁-C₆ straight, branched or cyclic hydrocarbon groups are preferred.

Examples of the divalent hydrocarbon group represented by R³⁰² include methylene, ethylene, propylene, butylene, and pentylene.

Examples of the monovalent hydrocarbon group represented by R³⁰³ or R³⁰⁶ include alkyl, alkenyl, and alkynyl groups, with the alkyl groups being preferred. Suitable alkyl groups include those exemplified for the monovalent hydrocarbon group represented by R³⁰¹ as well as n-undecyl, n-dodecyl, tridecyl, tetradecyl, and pentadecyl. Examples of the monovalent fluorinated hydrocarbon group represented by R³⁰³ or R³⁰⁶ include the foregoing monovalent hydrocarbon groups in which some or all carbon-bonded hydrogen atoms are substituted by fluorine atoms. In these groups, an ether bond (—O—) or carbonyl moiety (—C(═O)—) may intervene in a carbon-carbon bond as mentioned above.

Examples of the acid labile group represented by R³⁰³ include groups of the above formulae (L1) to (L4), C₄-C₂₀, preferably C₄-C₁₅ tertiary alkyl groups, trialkylsilyl groups in which each alkyl moiety has 1 to 6 carbon atoms, and C₄-C₂₀ oxoalkyl groups.

Examples of the (u+1)-valent hydrocarbon or fluorinated hydrocarbon group represented by R³⁰⁴ include the foregoing monovalent hydrocarbon or fluorinated hydrocarbon groups from which the number (u) of hydrogen atoms are eliminated.

Examples of the fluorinated hydrocarbon group represented by R^{305A} include the foregoing monovalent hydrocarbon groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the recurring units having formula (4) to (8) are shown below, but not limited thereto. Herein R^{F} is as defined above.

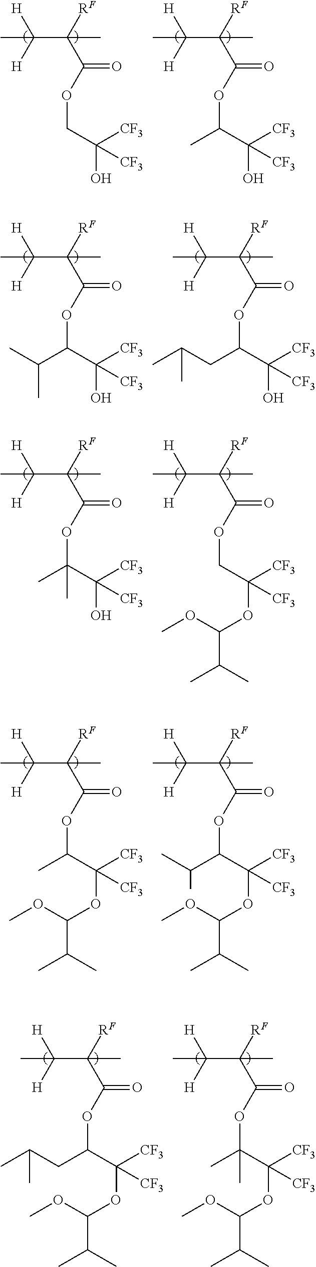

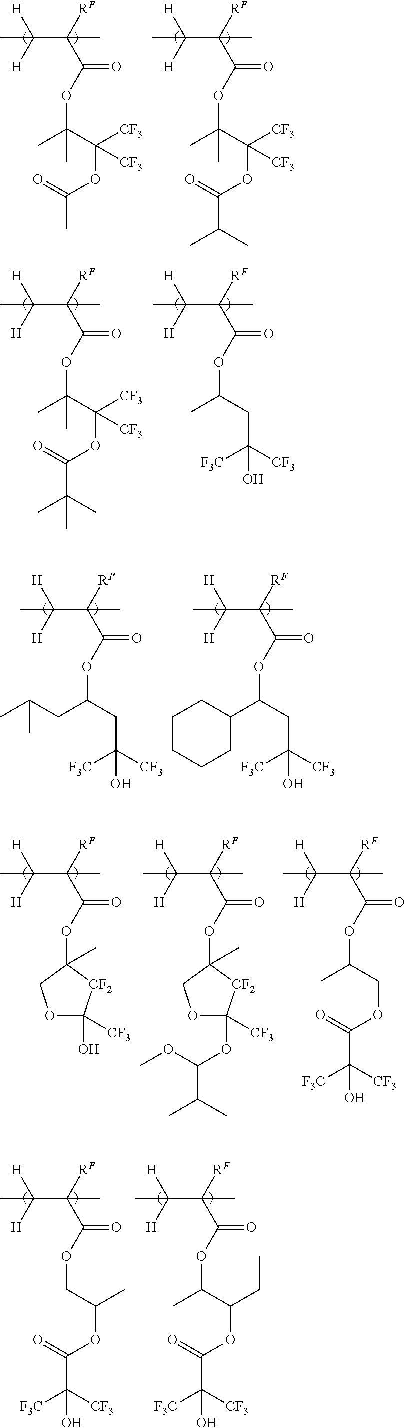

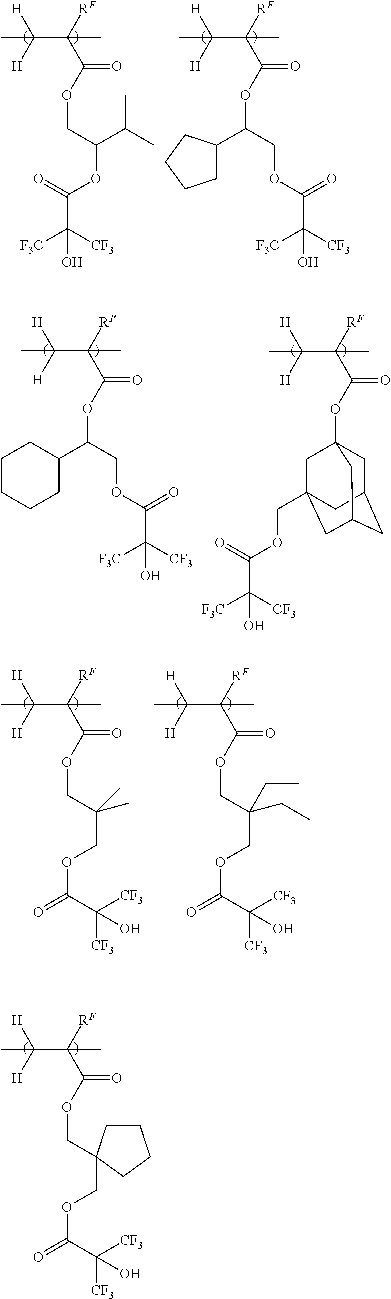

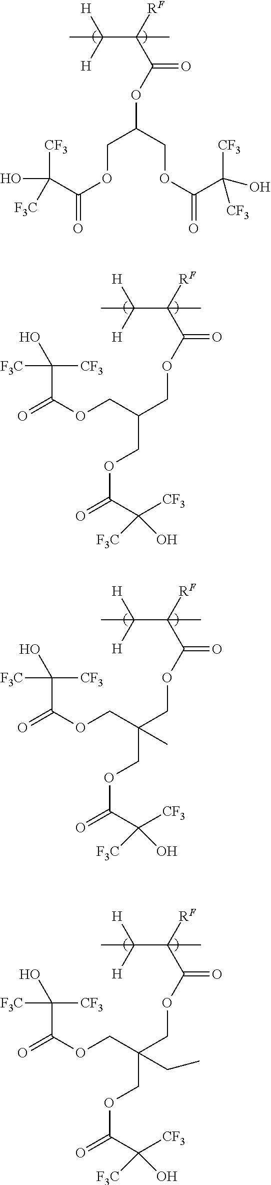

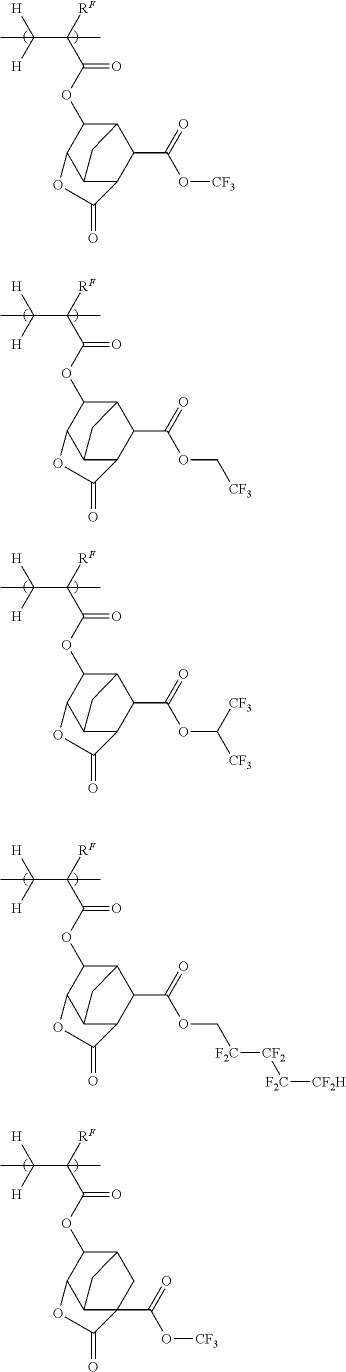

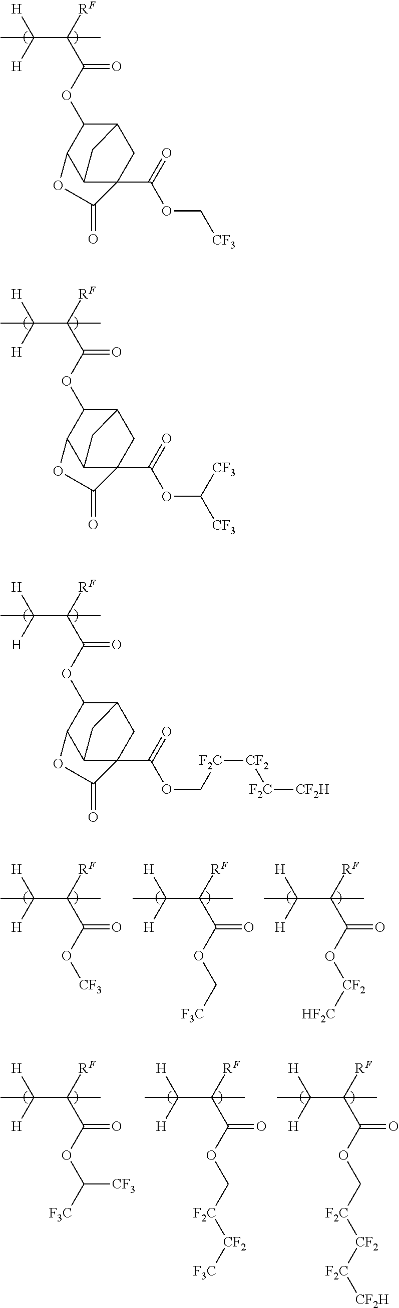

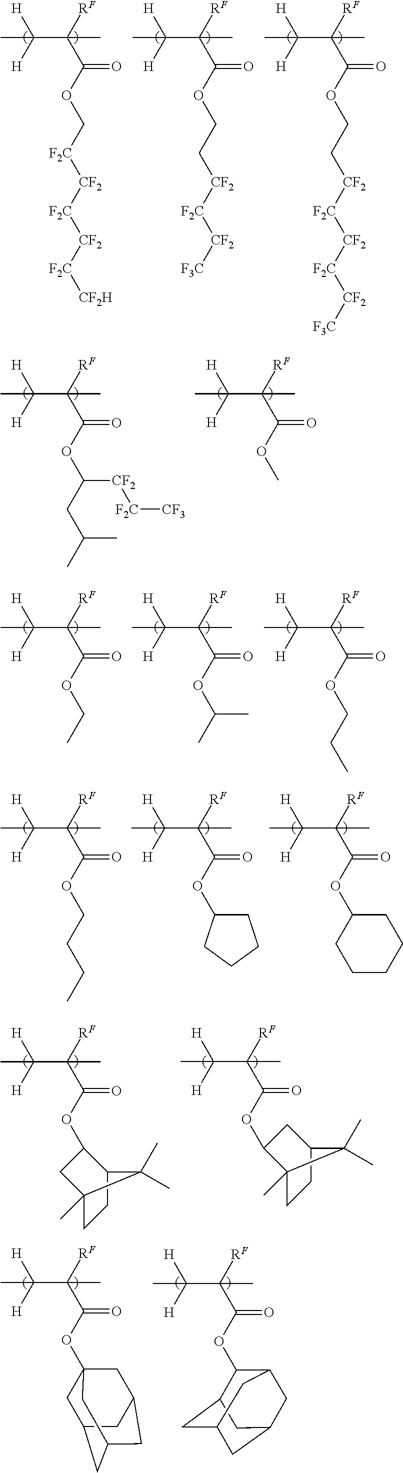

The polymeric surfactant may further contain recurring units other than the recurring units having formulae (4) to (8). Typical other recurring units are those derived from methacrylic acid and α-trifluoromethylacrylic acid derivatives. In the polymeric surfactant, the content of the recurring units having formulae (4) to (8) is preferably at least 20 mol %, more preferably at least 60 mol %, most preferably 100 mol % of the overall recurring units.

The polymeric surfactant preferably has a Mw of 1,000 to 500,000, more preferably 2,000 to 30,000. A polymeric surfactant having a Mw within the range exerts a satisfactory surface-modifying effect and causes no or few development defects. The Mw/Mn is preferably 1.0 to 2.0, more preferably 1.0 to 1.6.

For the surfactant which is insoluble or substantially insoluble in water and soluble in alkaline developer, reference may be made to JP-A 2008-122932, 2009-098638, 2009-191151, 2009-192784, 2009-276363, 2010-107695, 2010-134012, 2010-250105, and 2011-042789.

The surfactant (E) is preferably formulated in an amount of 0 to 20 parts by weight per 100 parts by weight of the base resin (C). When used, the amount of the surfactant is preferably at least 0.001 part, more preferably at least 0.01 part by weight and up to 15 parts, more preferably up to 10 parts by weight. The surfactant (E) may be used alone or in admixture.

### (F) Nitrogen-Containing Compound

While the resist composition essentially contains component (A) or quencher, a nitrogen-containing compound may be added as an additional quencher. Suitable nitrogen-containing compounds include primary, secondary and tertiary amine compounds, specifically amine compounds having a hydroxyl group, ether bond, ester bond, lactone ring, cyano group, or sulfonic acid ester bond, as described in JP-A 2008-111103, paragraphs [0146]-[0164], and primary and secondary amine compounds protected with a carbamate group, as described in JP 3790649.

Also a sulfonium salt of sulfonic acid having a nitrogen-containing substituent may be used as component (F). This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and 2012-046501, for example

The nitrogen-containing compound (F) is preferably formulated in an amount of 0.001 to 12 parts by weight, more preferably 0.01 to 8 parts by weight per 100 parts by weight of the base resin (C). The nitrogen-containing compound may be used alone or in admixture.

Process

A further embodiment of the invention is a resist pattern forming process comprising the steps of applying the resist composition defined above onto a substrate to form a resist film thereon, exposing the resist film patternwise to KrF or ArF excimer laser, EB or EUV, and developing the resist film in a developer to form a resist pattern.

Pattern formation using the resist composition of the invention may be performed by well-known lithography processes. In general, the resist composition is first applied onto a substrate such as a substrate for IC fabrication (e.g., Si, SiO₂, SiN, SiON, TiN, WSi, BPSG, SOG, organic antireflective coating, etc.) or a substrate for mask circuit fabrication (e.g., Cr, CrO, CrON, MoSi₂, SiO₂, etc.) by a suitable coating technique such as spin coating. The coating is prebaked on a hotplate at a temperature of 60 to 180° C. for 10 to 600 seconds, preferably at 70 to 150° C. for 15 to 300 seconds to form a resist film of 0.05 to 2 μm thick.

Then the resist film is exposed patternwise to KrF or ArF excimer laser, EUV or EB. On use of KrF or ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. In the case of immersion lithography, a protective film which is insoluble in water may be formed on the resist film.

While the water-insoluble protective film serves to prevent any components from being leached out of the resist film and to improve water slippage at the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, and more preferably at 80 to 140° C. for 1 to 3 minutes.

Finally, development is carried out using as the developer an aqueous alkaline solution, such as a 0.1 to 5 wt %, preferably 2 to 3 wt %, aqueous solution of tetramethylammonium hydroxide (TMAH), this being done by a conventional method such as dip, puddle, or spray development for a period of 0.1 to 3 minutes, and preferably 0.5 to 2 minutes. In this way the desired pattern is formed on the substrate.

Any desired step may be added to the pattern forming process. For example, after a resist film is formed, the step of rinsing with pure water (post-soaking) may be introduced to extract the acid generator or the like from the film surface or wash away particles. After exposure, the step of rinsing (post-soaking) may be introduced to remove any water remaining on the film after exposure.

A pattern may also be formed by a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, an alkaline aqueous solution is often used as the developer. Instead, the negative tone development technique wherein the unexposed region is developed and dissolved in an organic solvent is also applicable.

In the organic solvent development, the organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, phenyl acetate, propyl formate, butyl formate, isobutyl formate, pentyl formate, isopentyl formate, methyl valerate, methyl pentenoate, methyl crotonate, ethyl crotonate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture of two or more.

### EXAMPLES

Examples and Comparative Examples are given below by way of illustration and not by way of limitation. All parts are by weight (pbw). The composition of a copolymer is expressed by a molar ratio. THF is tetrahydrofuran, DMF is dimethylformamide, MIBK is methyl isobutyl ketone, and PGMEA is propylene glycol monomethyl ether acetate. Mw is measured by GPC versus polystyrene standards. Analytic instruments are as shown below.
IR: NICOLET 6700 by Thermo Fisher Scientific Inc.
¹H-NMR: ECA-500 by JEOL-Ltd.
MALDI-TOFMS: S3000 by JEOL Ltd.
   [1] Synthesis of quencher or onium salt

### Example 1-1

Synthesis of triphenylsulfonium bis(cyclohexanesulfonyl)(methanesulfonyl)methide (Q-A)

(1) Synthesis of bis(cyclohexanesulfonyl)(methylthio)methane (Intermediate A)

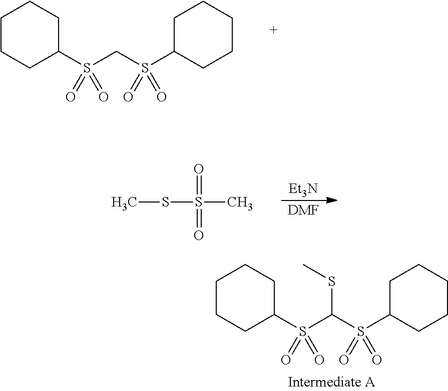

Under ice cooling, 4.5 g of S-methyl thiomethanesulfonate was added dropwise to a mixture of 9.2 g of bis(cyclohexanesulfonyl)methane, 6.1 g of triethylamine, and 28 g of dimethylformamide. After the solution was aged for 3 hours, 140 g of dilute hydrochloric acid was added for crystallization. The crystal precipitate was filtered, washed with ultrapure water and then with methanol, and dried in vacuum, obtaining 7.8 g (yield 74%) of the desired compound, Intermediate A.

(2) Synthesis of bis(cyclohexanesulfonyl)(methanesulfonyl)methane (Intermediate B)

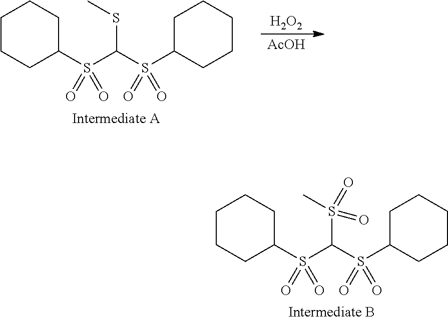

At room temperature, 3.9 g of 35 wt % aqueous hydrogen peroxide was added dropwise to a mixture of 5.6 g of Intermediate A and 28 g of acetic acid. The solution was aged at 60° C. for 2 hours. The reaction solution was cooled to room temperature. The resulting crystal precipitate was filtered and washed with water. The wet crystal was dried in vacuum, obtaining 4.4 g of the desired compound, Intermediate B. The product was used in the subsequent step without further purification.

(3) Synthesis of triphenylsulfonium bis(cyclohexanesulfonyl)(methanesulfonyl)methide (Q-A)

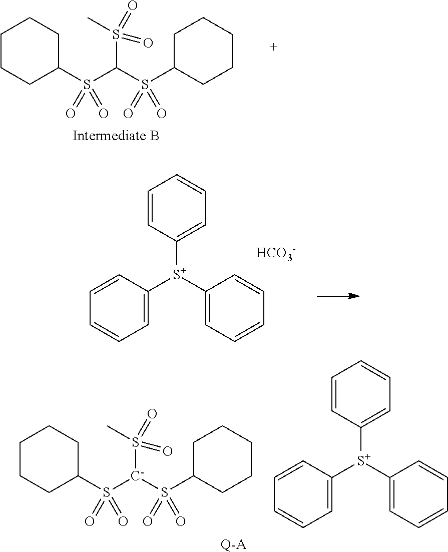

A mixture of 4.4 g of Intermediate B, 31 g of 20 wt % triphenylsulfonium hydrogencarbonate aqueous solution (prepared according to the teaching of WO 2015/019983), and 30 g of MIBK was aged at room temperature for 1 hour. The organic layer was taken out and washed with ultrapure water. The organic layer as washed was concentrated under reduced pressure, purified by column chromatography, and recrystallized from MIBK. The resulting crystal was recovered and dried in vacuum, obtaining 3.8 g (yield 59%) of the target compound, triphenylsulfonium bis(cyclohexanesulfonyl)(methanesulfonyl)methide (Q-A).

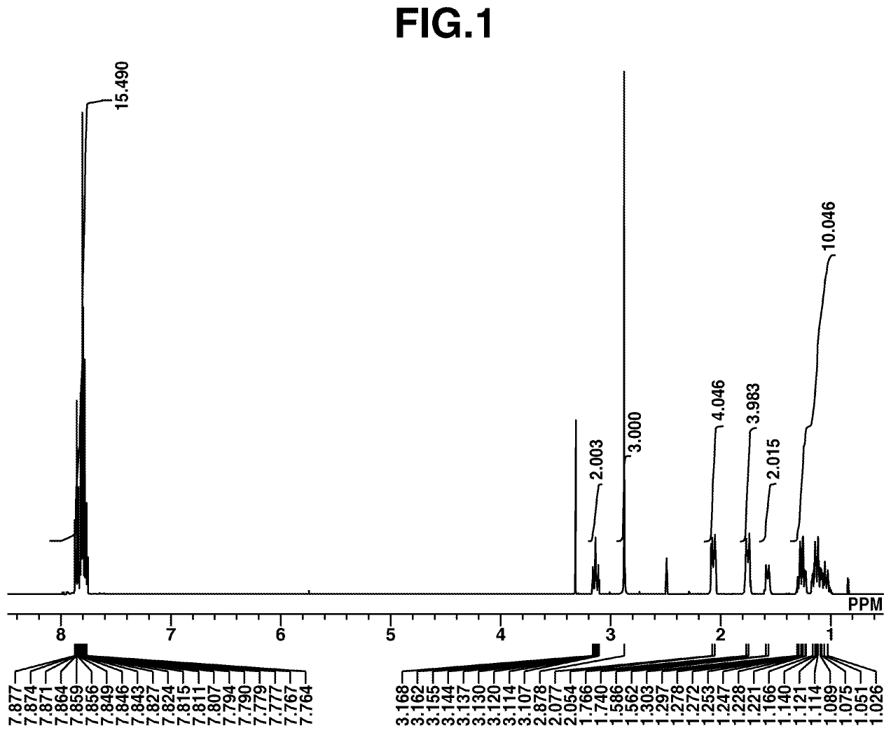

The target compound was analyzed by spectroscopy. The NMR spectrum, ¹H-NMR in DMSO-d₆ is shown in FIG. 1. In ¹H-NMR analysis, minute amounts of residual solvent (MIBK) and water were observed.

### IR (D-ATR):

2928, 2859, 1478, 1447, 1306, 1289, 1259, 1132, 1110, 1076, 1008, 991, 952, 763, 755, 750, 687, 657, 609, 544, 528, 514, 496 cm⁻¹

### MALDI-TOFMS:

Positive [M⁺] 263 (corresponding to C₁₈H₁₅S⁺)

Negative [M⁻] 385 (corresponding to C₁₄H₂₅O₆S₃⁻)

### Example 1-2

Synthesis of bis(4-tert-butyl)diphenyliodonium bis(cyclohexanesulfonyl) (methanesulfonyl)methane (Q-B)

(1) Synthesis of benzyltrimethylammonium bis(cyclohexanesulfonyl) (methanesulfonyl)methane (Intermediate C)

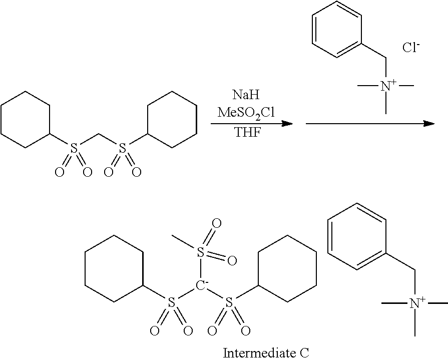 [SMILES: O=S(=O)(CS(=O)(=O)C1CCCCC1)C1CCCCC1 ; C[N+](C)(C)Cc1ccccc1 ; [Cl-] ; CS(=O)(=O)[C-](S(=O)(=O)C1CCCCC1)S(=O)(=O)C1CCCCC1 ; C[N+](C)(C)Cc1ccccc1 ; "THF"]

A solution of 50.02 g of bis(cyclohexanesulfonyl)methane in 250 g of THF was added dropwise to a solution of 14.28 g of sodium hydride (55 wt % in liquid paraffin) in 50 g of THF at room temperature. Successively, 24.18 g of methanesulfonyl chloride was added dropwise to the solution, which was stirred at 50° C. for 15 hours. After 355 g of 5 wt % hydrochloric acid was added to the solution to quench the reaction, dichloromethane and water were added to the reaction solution, which was allowed to separate. The organic layer was taken out, 28.51 g of 25 wt % sodium hydroxide aqueous solution was added, and the solution was stirred. Subsequently, 36.10 g of benzyltrimethylammonium chloride was added to the solution, which was allowed to separate. The organic layer was taken out, washed with ultrapure water, concentrated under reduced pressure, and crystallized from MIBK. The resulting crystal was recovered and dried in vacuum, obtaining 35.81 g of Intermediate C (two-step yield 40.9%).

(2) Synthesis of bis(4-tert-butyl)diphenyliodonium bis(cyclohexanesulfonyl) (methanesulfonyl)methane (Q-B)

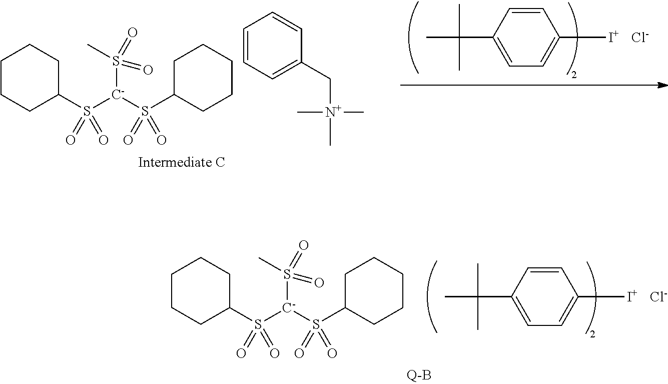

At room temperature, 5 g of Intermediate C, 5 g of bis(4-tert-butyl)diphenyliodonium chloride, 45 g of MIBK, and 10 g of water were mixed and stirred for 30 minutes. The organic layer was taken out, washed with water, and concentrated under reduced pressure. Diisopropyl ether was added to the concentrate, from which crystals precipitated out. The crystal precipitate was filtered and dried in vacuum at 50° C., obtaining 7 g (yield 90%) of the target compound, bis(4-tert-butyl)diphenyliodonium bis(cyclohexanesulfonyl) (methanesulfonyl)methane (Q-B) in white solid form.

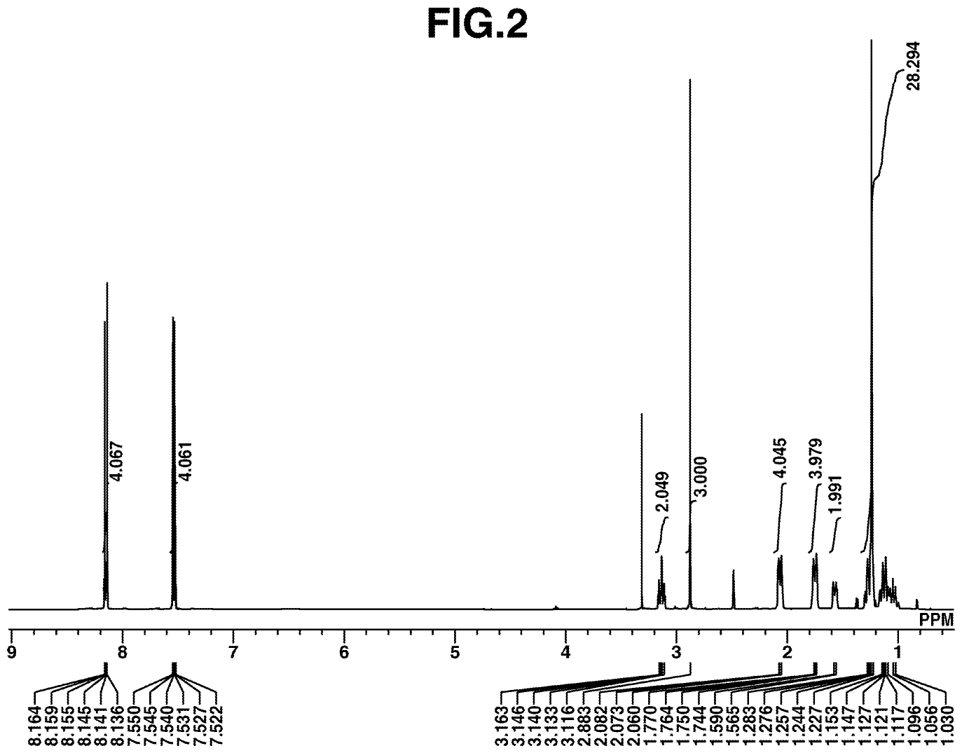

The target compound was analyzed by spectroscopy. The NMR spectrum, ¹H-NMR in DMSO-d₆ is shown in FIG. 2. In ¹H-NMR analysis, minute amounts of residual solvents (MIBK and diisopropyl ether) and water were observed.

### IR (D-ATR):

2965, 2935, 2856, 1483, 1453, 1290, 1270, 1246, 1215, 1126, 1093, 1011, 988, 951, 841, 655, 605, 542, 526, 518 cm⁻¹

### MALDI-TOFMS:

Positive [M⁺] 393 (corresponding to C₂₀H₂₆I⁺)

Negative [M⁻] 385 (corresponding to C₁₄H₂₅O₆S₃⁻)

### Example 1-3

Synthesis of 5-phenyldibenzothiophenium bis(cyclohexanesulfonyl) (methanesulfonyl)methane (Q-C)

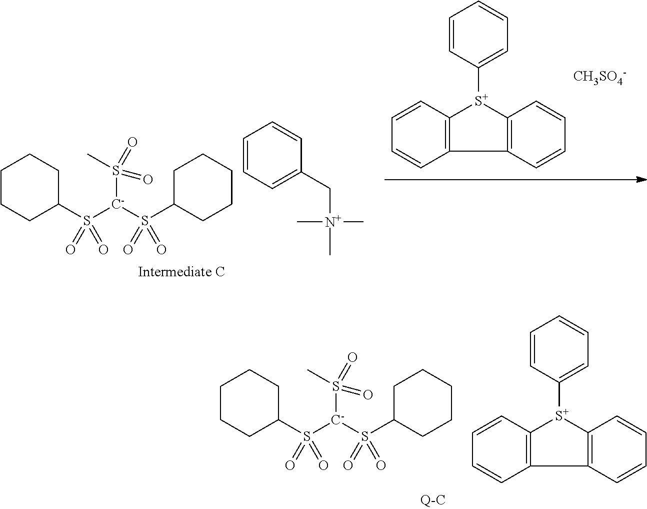

At room temperature, 5 g of Intermediate C, 4 g of 5-phenyldibenzothiophenium methylsulfate, 28 g of methylene chloride, and 24 g of water were mixed and stirred for 30 minutes. The organic layer was taken out, washed with water, and concentrated under reduced pressure. Thereafter, 50 g of MIBK was added to the concentrate, which was washed with water and concentrated under reduced pressure. Diisopropyl ether was added to the concentrate, from which crystals precipitated out. The crystal precipitate was filtered and dried in vacuum at 50° C., obtaining 4 g (yield 63%) of the target compound, 5-phenyldibenzothiophenium bis(cyclohexanesulfonyl)(methanesulfonyl)methane (Q-C) in white solid form.

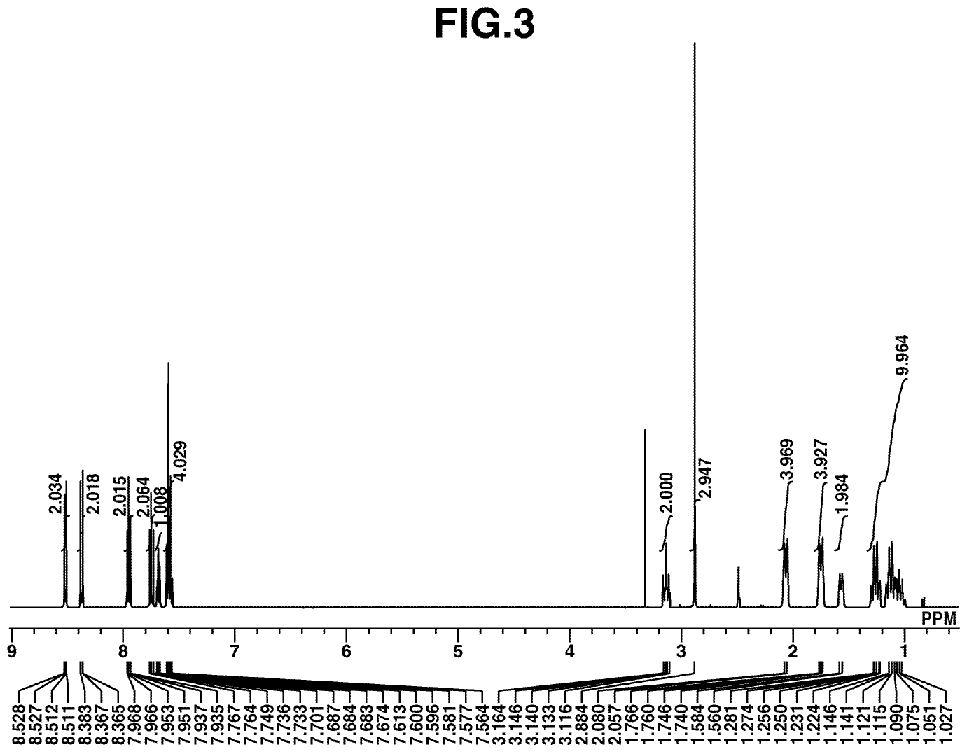

The target compound was analyzed by spectroscopy. The NMR spectrum, ¹H-NMR in DMSO-d₆ is shown in FIG. 3. In ¹H-NMR analysis, minute amounts of residual solvent (MIBK) and water were observed.

### IR (D-ATR):

2936, 2921, 2849, 1448, 1307, 1287, 1244, 1113, 1096, 1010, 987, 957, 939, 770, 759, 661, 580, 541, 526 cm⁻¹

### MALDI-TOFMS:

Positive [M⁺] 261 (corresponding to C₁₈H₁₃S⁺)

Negative [M⁻] 385 (corresponding to C₁₄H₂₅O₆S₃⁻)

### Example 1-4

Synthesis of 4-(2-methoxyethoxy)naphthalene-1-tetrahydrothiopyranium bis(cyclohexanesulfonyl)(methanesulfonyl)methane (Q-D)

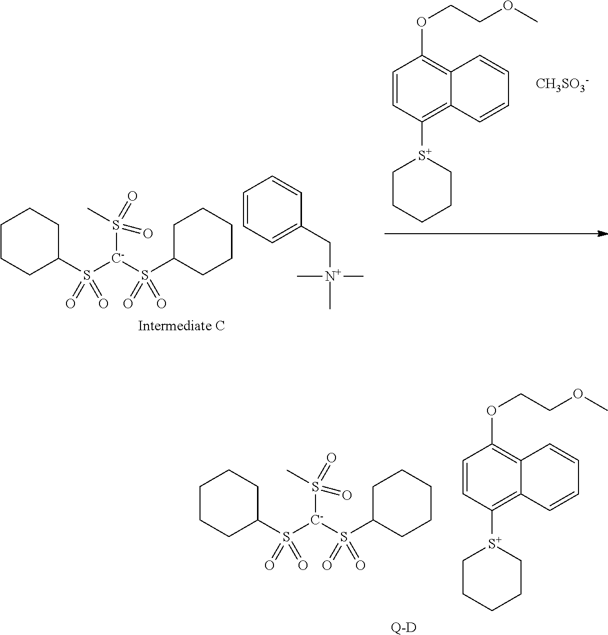

At room temperature, 5 g of Intermediate C, 50 g of 15 wt % aqueous solution of 4-(2-methoxyethoxy)naphthalene-1-tetrahydrothiopyranium methanesulfonate, and 60 g of methylene chloride were mixed and stirred for 30 minutes. The organic layer was taken out, washed with water, and concentrated under reduced pressure. Thereafter, 50 g of MIBK was added to the concentrate, which was concentrated under reduced pressure again. Diisopropyl ether was added to the concentrate, from which crystals precipitated out. The crystal precipitate was filtered and dried in vacuum at 50° C., obtaining 6 g (yield 96%) of the target compound, 4-(2-methoxyethoxy)naphthalene-1-tetrahydrothiopyranium bis(cyclohexanesulfonyl)(methanesulfonyl)methane (Q-D) in white solid form.

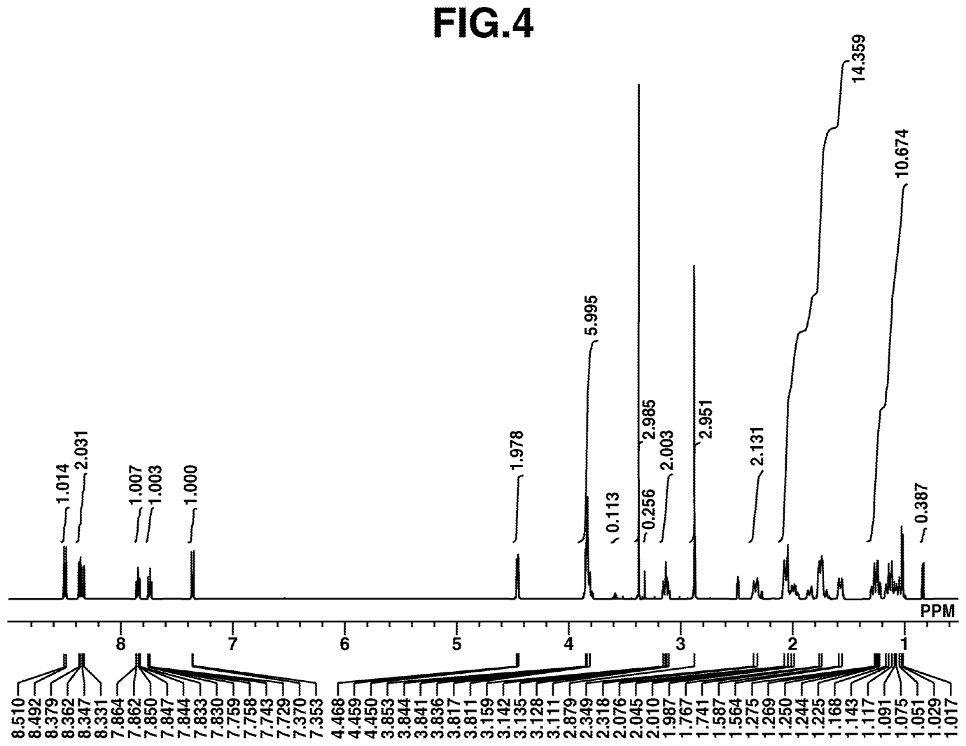

The target compound was analyzed by spectroscopy. The NMR spectrum, ¹H-NMR in DMSO-d₆ is shown in FIG. 4. In ¹H-NMR analysis, minute amounts of residual solvents (MIBK, diisopropyl ether) and water were observed.

### IR (D-ATR):

2937, 2859, 1592, 1571, 1509, 1462, 1446, 1431, 1377, 1325, 1291, 1277, 1249, 1220, 1209, 1134, 1105, 1083, 1014, 991, 967, 957, 853, 819, 782, 771, 610, 545, 527, 518 cm⁻¹

### MALDI-TOFMS:

Positive [M⁺] 303 (corresponding to C₁₈H₂₃O₂S⁺)

Negative [M⁻] 385 (corresponding to C₁₄H₂₅O₆S₃⁻)

### Example 1-5

Synthesis of 4-tert-butylnaphthalene-1-tetrahydrothiophenium bis(cyclohexanesulfonyl)(methanesulfonyl)methane (Q-E)

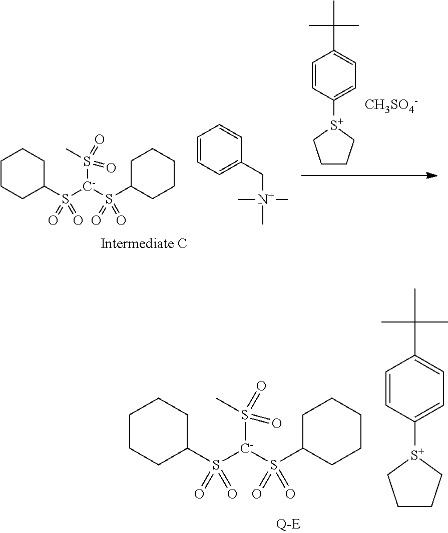

At room temperature, 5 g of Intermediate C, 110 g of 6 wt % aqueous solution of 4-tert-butylnaphthalene-1-tetrahydrothiophenium methylsulfate, and 50 g of methylene chloride were mixed and stirred for 30 minutes. The organic layer was taken out, washed with water, and concentrated under reduced pressure. The concentrate was purified by silica gel chromatography. PGMEA was added for crystallization, followed by addition of diisopropyl ether and 1 hour of stirring. The crystal precipitate was filtered and dried in vacuum at 50° C., obtaining 3 g (yield 50%) of the target compound, 4-tert-butylnaphthalene-1-tetrahydrothiophenium bis(cyclohexanesulfonyl)(methanesulfonyl)methane (Q-E) in white solid form.

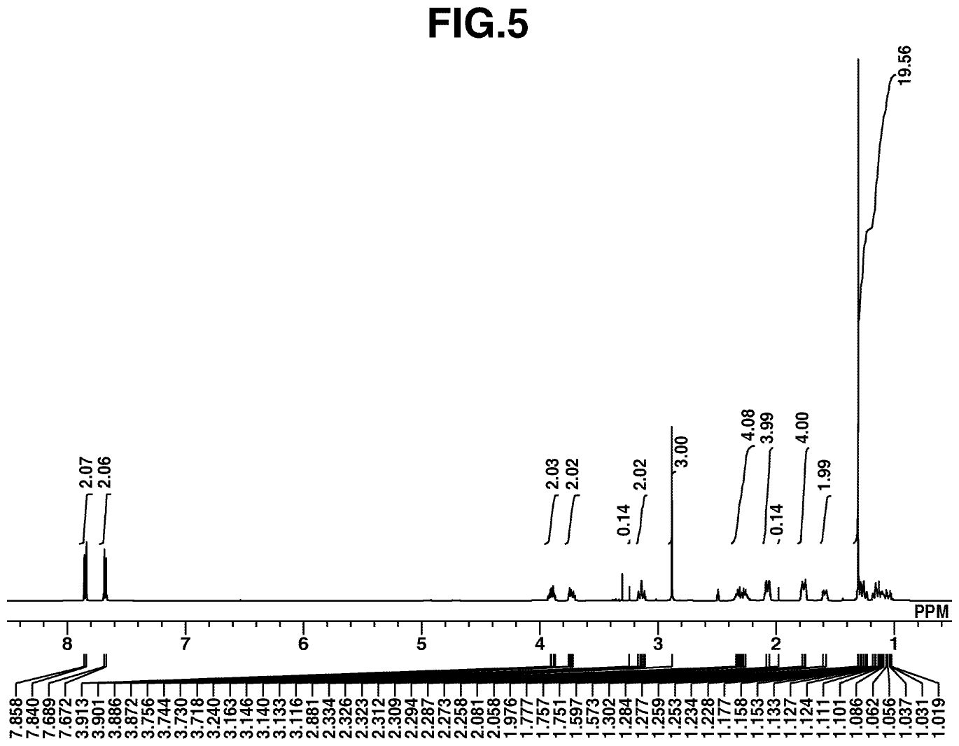

The target compound was analyzed by spectroscopy. The NMR spectrum, ¹H-NMR in DMSO-d₆ is shown in FIG. 5. In ¹H-NMR analysis, minute amounts of residual solvent (PGMEA) and water were observed.

### IR (D-ATR):

2931, 2851, 1449, 1291, 1272, 1255, 1134, 1110, 1011, 992, 962, 945, 754, 663, 607, 581, 557, 545, 527, 517 cm⁻¹

### MALDI-TOFMS:

Positive [M⁺] 221 (corresponding to C₁₄H₂₁S⁺)

Negative [M⁻] 385 (corresponding to C₁₄H₂₅O₆S₃⁻)

### [2] Synthesis of Polymers

### Synthesis Example 1

Synthesis of Polymer P1

In nitrogen atmosphere, 19 g of 1-ethylcyclopentyl methacrylate, 17 g of 2-oxotetrahydrofuran-3-yl mechacrylate, 0.48 g of dimethyl 2,2′-azobis(2-methylpropionate) (V-601 by Wako Pure Chemical Corp.), 0.41 g of 2-mercaptoethanol, and 50 g of methyl ethyl ketone were fed into a funnel to form a monomer/initiator solution. A flask in nitrogen atmosphere was charged with 23 g of methyl ethyl ketone, which was heated at 80° C. with stirring. With stirring, the monomer/initiator solution was added dropwise to the flask over 4 hours. After the completion of dropwise addition, the polymerization solution was continuously stirred for 2 hours while maintaining its temperature at 80° C. After the polymerization solution was cooled to room temperature, it was added dropwise to 640 g of methanol under vigorous stirring. The precipitate was collected by filtration, washed twice with 240 g of methanol, and vacuum dried at 50° C. for 20 hours, obtaining a polymer P1 in white powder form. Amount 36 g, yield 90%. On GPC analysis, Polymer P1 had a Mw of 8,755 and a Mw/Mn of 1.94.

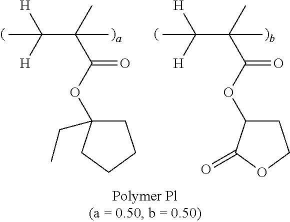

### Synthesis Examples 2 to 12

Synthesis of Polymers P2 to P12

Polymers consisting of units in Table 1 were synthesized by the same procedure as in Synthesis Example 1 aside from changing the type and amount of monomers.

Table 1 shows the proportion (in molar ratio) of units incorporated in these polymers, and Tables 2 to 4 show the structure of recurring units.

TABLE 1

| | Unit 1 | | Unit 2 | | Unit 3 | | Unit 4 | | | |
|---|---|---|---|---|---|---|---|---|---|---|
| Polymer | (proportion) | | (proportion) | | (proportion) | | (proportion) | | Mw | Mw/Mn |
| P1 | A-1 | (0.50) | B-1 | (0.50) | — | — | — | — | 8,755 | 1.94 |
| P2 | A-2 | (0.50) | B-2 | (0.40) | B-4 | (0.10) | — | — | 9,330 | 2.03 |
| P3 | A-1 | (0.50) | B-1 | (0.30) | B-3 | (0.20) | — | — | 9,000 | 2.01 |
| P4 | A-1 | (0.30) | A-4 | (0.20) | B-3 | (0.50) | — | — | 9,560 | 1.88 |
| P5 | A-1 | (0.30) | A-2 | (0.20) | B-1 | (0.40) | B-4 | (0.10) | 8,320 | 1.98 |
| P6 | A-1 | (0.30) | A-2 | (0.20) | B-2 | (0.40) | B-4 | (0.10) | 9,280 | 2.08 |
| P7 | A-4 | (0.50) | B-3 | (0.50) | — | — | — | — | 8,980 | 1.79 |
| P8 | A-1 | (0.30) | A-3 | (0.20) | B-3 | (0.40) | B-4 | (0.10) | 8,950 | 1.82 |
| P9 | A-2 | (0.30) | B-2 | (0.40) | B-5 | (0.20) | PM-1 | (0.10) | 8,530 | 2.11 |
| P10 | A-1 | (0.50) | B-6 | (0.40) | PM-2 | (0.10) | — | — | 9,690 | 1.92 |
| P11 | A-5 | (0.30) | B-1 | (0.50) | B-6 | (0.20) | — | — | 10,200 | 1.71 |
| P12 | A-6 | (0.30) | B-1 | (0.50) | B-6 | (0.20) | — | — | 11,210 | 2.06 |

TABLE 2

| | |
|---|---|
| 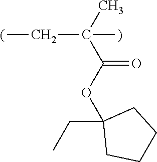 | A-1 |
| 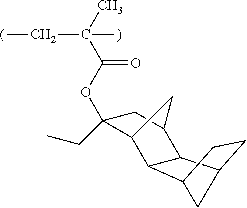 | A-2 |
| 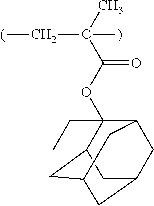 | A-3 |
| 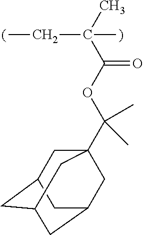 | A-4 |
| 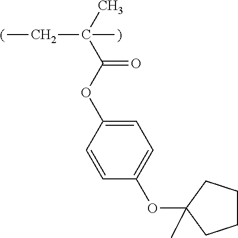 | A-5 |
| 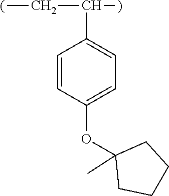 | A-6 |

TABLE 3

| | |
|---|---|
| 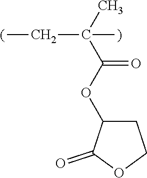 | B-1 |
| 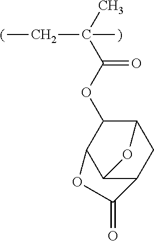 | B-2 |
| 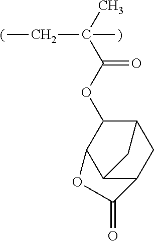 | B-3 |
| 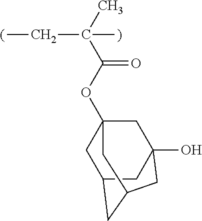 | B-4 |
| 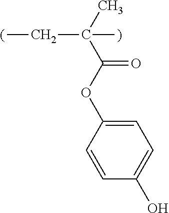 | B-5 |
| 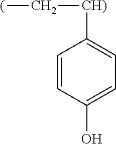 | B-6 |

TABLE 4

| | |
|---|---|
| 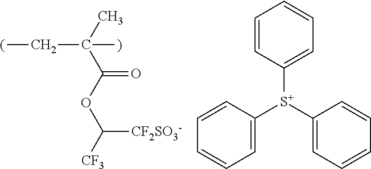 | PM-1 |
| 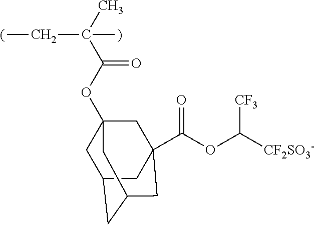 | PM-2 |
| 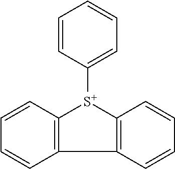 [SMILES: c1ccc(-[s+]2c3ccccc3c3ccccc32)cc1] | |

### [3] Preparation of Resist Composition

### Examples 2-1 to 2-16 and Comparative Examples 1-1 to 1-8

A resist composition in solution form was prepared by dissolving a quencher (inventive Q-A to Q-E, comparative Q-1 to Q-4), polymer (P1 to P12), photoacid generator (PAG-X), and surfactant (SF-1) in an organic solvent containing 0.01 wt % of surfactant A in accordance with the formulation shown in Tables 5 and 6, and filtering through a Teflon® filter with a pore size of 0.2 μm.

The solvents, photoacid generator PAG-X, surfactant SF-1, Surfactant A, and comparative quenchers Q-1 to Q-4 in Tables 5 and 6 are identified below.

### Solvents

PGMEA (propylene glycol monomethyl ether acetate)

GBL (γ-butyrolactone)

### Photoacid Generator PAG-X:

triphenylsulfonium 2-(adamantane-1-carbonyloxy)-1,1,1,3,3,3-pentafluoropropane-1-sulfonate

### Comparative Quenchers

Q-1: 2-(4-morpholinyl)ethyl laurate

Q-2: triphenylsulfonium 10-camphorsulfonate

Q-3: triphenylsulfonium salicylate

Q-4: triphenylsulfonium tris(trifluoromethanesulfonyl)methide

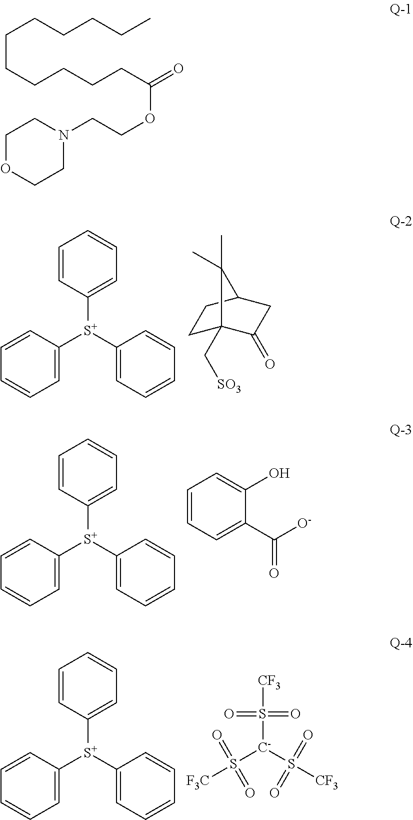

### Alkali-Soluble Surfactant SF-1:

poly(2,2,3,3,4,4,4-heptafluoro-1-isobutyl-1-butyl methacrylate/9-(2,2,2-trifluoro-1-trifluoroethyloxycarbonyl)-4-oxatricyclo[4.2.1.0^{3,7}]nonan-5-on-2-yl methacrylate)
Mw=7,700
Mw/Mn=1.82

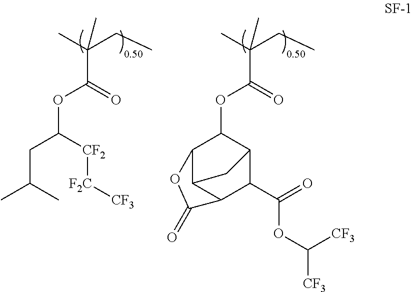

### Surfactant A:

3-methyl-3-(2,2,2-trifluoroethoxymethyl)oxetane/tetrahydrofuran/2,2-dimethyl-1,3-propane diol copolymer (Omnova Solutions, Inc.)

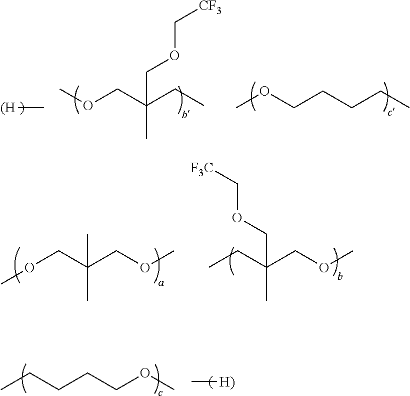

a:(b+b′):(c+c′)=1:4-7:0.01-1 (molar ratio)
Mw=1,500

TABLE 5

| | | Resist | Polymer | Acid generator | Quencher | Surfactant | Solvent 1 | Solvent 2 |
|---|---|---|---|---|---|---|---|---|
| | | composition | (pbw) | (pbw) | (pbw) | (pbw) | (pbw) | (pbw) |
| Example | 2-1 | R-01 | P-1 | PAG-X | Q-A | SF-1 | PGMEA | GBL |
| | | | (80) | (7.6) | (5.0) | (3.0) | (1,728) | (192) |
| | 2-2 | R-02 | P-2 | PAG-X | Q-A | SF-1 | PGMEA | GBL |
| | | | (80) | (7.6) | (5.0) | (3.0) | (1,728) | (192) |
| | 2-3 | R-03 | P-3 | PAG-X | Q-A | SF-1 | PGMEA | GBL |
| | | | (80) | (7.6) | (5.0) | (3.0) | (1,728) | (192) |
| | 2-4 | R-04 | P-4 | PAG-X | Q-A | SF-1 | PGMEA | GBL |
| | | | (80) | (7.6) | (5.0) | (3.0) | (1,728) | (192) |
| | 2-5 | R-05 | P-5 | PAG-X | Q-A | SF-1 | PGMEA | GBL |
| | | | (80) | (7.6) | (5.0) | (3.0) | (1,728) | (192) |
| | 2-6 | R-06 | P-6 | PAG-X | Q-A | SF-1 | PGMEA | GBL |
| | | | (80) | (7.6) | (5.0) | (3.0) | (1,728) | (192) |
| | 2-7 | R-07 | P-7 | PAG-X | Q-A | SF-1 | PGMEA | GBL |
| | | | (80) | (7.6) | (5.0) | (3.0) | (1,728) | (192) |
| | 2-8 | R-08 | P-8 | PAG-X | Q-A | SF-1 | PGMEA | GBL |
| | | | (80) | (7.6) | (5.0) | (3.0) | (1,728) | (192) |
| | 2-9 | R-09 | P-9 | PAG-X | Q-A | SF-1 | PGMEA | GBL |
| | | | (80) | (7.6) | (5.0) | (3.0) | (1,728) | (192) |
| | 2-10 | R-10 | P-10 | PAG-X | Q-A | SF-1 | PGMEA | GBL |
| | | | (80) | (7.6) | (5.0) | (3.0) | (1,728) | (192) |
| | 2-11 | R-11 | P-11 | PAG-X | Q-A | SF-1 | PGMEA | GBL |
| | | | (80) | (7.6) | (5.0) | (3.0) | (1,728) | (192) |
| | 2-12 | R-12 | P-12 | PAG-X | Q-A | SF-1 | PGMEA | GBL |
| | | | (80) | (7.6) | (5.0) | (3.0) | (1,728) | (192) |
| | 2-13 | R-13 | P-9 | PAG-X | Q-B | SF-1 | PGMEA | GBL |
| | | | (80) | (7.6) | (6.0) | (3.0) | (1,728) | (192) |
| | 2-14 | R-14 | P-9 | PAG-X | Q-C | SF-1 | PGMEA | GBL |
| | | | (80) | (7.6) | (5.0) | (3.0) | (1,728) | (192) |
| | 2-15 | R-15 | P-1 | PAG-X | Q-D | SF-1 | PGMEA | GBL |
| | | | (80) | (7.6) | (5.3) | (3.0) | (1,728) | (192) |
| | 2-16 | R-16 | P-1 | PAG-X | Q-E | SF-1 | PGMEA | GBL |
| | | | (80) | (7.6) | (4.7) | (3.0) | (1,728) | (192) |

TABLE 6

| | | Resist | Polymer | Acid generator | Quencher | Surfactant | Solvent 1 | Solvent 2 |
|---|---|---|---|---|---|---|---|---|
| | | composition | (pbw) | (pbw) | (pbw) | (pbw) | (pbw) | (pbw) |
| Comparative Example | 1-1 | CR-01 | P-1 | PAG-X | Q-1 | SF-1 | PGMEA | GBL |
| | | | (80) | (7.6) | (1.3) | (3.0) | (1,728) | (192) |
| | 1-2 | CR-02 | P-1 | PAG-X | Q-2 | SF-1 | PGMEA | GBL |
| | | | (80) | (7.6) | (3.8) | (3.0) | (1,728) | (192) |
| | 1-3 | CR-03 | P-2 | PAG-X | Q-3 | SF-1 | PGMEA | GBL |
| | | | (80) | (7.6) | (3.1) | (3.0) | (1,728) | (192) |
| | 1-4 | CR-04 | P-2 | PAG-X | Q-4 | SF-1 | PGMEA | GBL |
| | | | (80) | (7.6) | (5.2) | (3.0) | (1,728) | (192) |
| | 1-5 | CR-05 | P-9 | PAG-X | Q-1 | SF-1 | PGMEA | GBL |
| | | | (80) | (7.6) | (1.3) | (3.0) | (1,728) | (192) |
| | 1-6 | CR-06 | P-9 | PAG-X | Q-2 | SF-1 | PGMEA | GBL |
| | | | (80) | (7.6) | (3.8) | (3.0) | (1,728) | (192) |
| | 1-7 | CR-07 | P-12 | PAG-X | Q-3 | SF-1 | PGMEA | GBL |
| | | | (80) | (7.6) | (3.1) | (3.0) | (1,728) | (192) |
| | 1-8 | CR-08 | P-12 | PAG-X | Q-4 | SF-1 | PGMEA | GBL |
| | | | (80) | (7.6) | (5.2) | (3.0) | (1,728) | (192) |

### [4] Evaluation of Resist Composition: ArF Lithography Test #1

### Examples 3-1 to 3-10 and Comparative Examples 2-1 to 2-4

An antireflective coating solution (ARC29A, Nissan Chemical Corp.) was coated on a silicon substrate and baked at 200° C. for 60 seconds to form an antireflective coating of 100 nm thick. Each of the resist compositions (R-01 to R-08, R-15, R-16, CR-01 to CR-04) was spin coated on the substrate and prebaked on a hotplate at 90° C. for 60 seconds to form a resist film of 90 nm thick. The resist film was exposed by ArF immersion lithography using an ArF excimer laser scanner (NSR-S610C by Nikon Corp., NA 1.30, quadrupole, 6% halftone phase shift mask). Water was used as the immersion liquid. After exposure, the resist film was baked (PEB) at the temperature shown in Table 6 for 60 seconds and developed in a 2.38 wt % TMAH aqueous solution for 60 seconds, forming a 1:1 line-and-space (L/S) pattern of 40 nm.

### Evaluation of Sensitivity

The L/S pattern was observed under an electron microscope. The optimum dose (Eop) is a dose (mJ/cm²) which provides a pattern having a line width of 40 nm.

### Evaluation of LWR

With respect to the L/S pattern formed by exposure in the optimum dose, the roughness of line sidewall was evaluated. The space width was measured at longitudinally spaced apart 30 points, from which a 3-fold value (30) of standard deviation (o) was determined and reported as LWR. A smaller value of LWR indicates that the line pattern has little or no fluctuation and is more satisfactory.

### Evaluation of MEF

Exposure was made through a mask having a fixed pitch of 80 nm and a varying line width in a range from 38 nm to 42 nm by an increment of 1 nm, scaled as on-wafer size at the optimum dose. The size of the pattern transferred to the wafer was measured. With respect to the line width, the size of the transferred pattern is plotted relative to the mask design size, and a gradient is computed by linear approximation, and reported as MEF. A smaller value of MEF indicates reduced influence of a finish error of the mask pattern and is better.

### Evaluation of Collapse Limit

In the process, the line size was reduced as the exposure dose was increased. The minimum size (nm) above which lines can be resolved without collapse is determined and reported as collapse limit. A smaller value indicates greater collapse resistance and is preferable.

The results are shown in Table 7.

TABLE 7

| | | Resist | PEB temp. | Eop | LWR | | Collapse limit |
|---|---|---|---|---|---|---|---|
| | | composition | (° C.) | (mJ/cm²) | (nm) | MEF | (nm) |
| Example | 3-1 | R-01 | 85 | 38 | 2.8 | 2.6 | 28 |
| | 3-2 | R-02 | 85 | 35 | 3.1 | 2.4 | 31 |
| | 3-3 | R-03 | 75 | 39 | 2.9 | 2.2 | 29 |
| | 3-4 | R-04 | 95 | 41 | 3.0 | 2.4 | 31 |
| | 3-5 | R-05 | 85 | 37 | 2.9 | 2.3 | 30 |
| | 3-6 | R-06 | 85 | 38 | 3.0 | 2.4 | 30 |
| | 3-7 | R-07 | 90 | 42 | 3.1 | 2.2 | 32 |
| | 3-8 | R-08 | 90 | 41 | 2.9 | 2.3 | 31 |
| | 3-9 | R-15 | 85 | 43 | 7.8 | 2.3 | 27 |
| | 3-10 | R-16 | 85 | 42 | 2.7 | 2.4 | 27 |
| Comparative | 2-1 | CR-01 | 85 | 38 | 4.1 | 3.1 | 43 |
| Example | 2-2 | CR-02 | 85 | 34 | 3.6 | 3.9 | 40 |
| | 2-3 | CR-03 | 85 | 37 | 3.9 | 3.7 | 38 |

| | | |
|---|---|---|
| 2-4 | CR-04 | Not resolved |

It is evident from the data of Table 7 that the resist compositions within the scope of the invention form, through alkaline development, positive patterns having improved LWR, MEF and collapse resistance. They are thus best suited as the ArF immersion lithography material.

It is noted that no pattern was observed in Comparative Example 2-4. This indicates that Q-4, though used as a quencher, functions as an acid generator. Although Q-4 has a methide anion structure like the inventive onium salt, it generates an acid having an extremely high acidity due to the influence of fluorine atoms contained therein. Thus the influence of Q-4 on lithography performance is utterly different from the inventive onium salt.

### [5] Evaluation of Resist Composition: ArF Lithography Test #2

### Examples 4-1 to 4-10 and Comparative Examples 3-1 to 3-4

On a silicon wafer, a spin-on carbon film ODL-50 (Shin-Etsu Chemical Co., Ltd.) having a carbon content of 80 wt % was deposited to a thickness of 200 nm and a silicon-containing spin-on hard mask SHB-A940 having a silicon content of 43 wt % was deposited thereon to a thickness of 35 nm. On this substrate for trilayer process, each of the resist compositions (R-01 to R-08, R-15, R-16, CR-01 to CR-04) was spin coated and baked on a hotplate at 100° C. for 60 seconds to form a resist film of 90 nm thick. Using an ArF excimer laser immersion lithography scanner NSR-610C (Nikon Corp., NA 1.30, a 0.98/0.74, cross-pole opening 35 deg.), pattern exposure was performed through a mask with a varying exposure dose and focus. After exposure, the resist film was baked (PEB) at the temperature shown in Table 7 for 60 seconds and developed in butyl acetate for 30 seconds.

### Evaluation of Sensitivity

The mask used herein is a binary mask having an on-mask design corresponding to a 55 nm dot/90 nm pitch pattern (actual on-mask size is 4 times because of ¼ image reduction projection exposure). The hole pattern printed on the resist through pattern reversal was observed under an electron microscope. The optimum dose (Eop) was the dose (mJ/cm²) that gave a hole inner diameter of 50 nm.

### Evaluation of CDU

A hole pattern resulted from image reversal by organic solvent development. By observation under TDSEM S-9380 (Hitachi High-Technologies Corp.), the diameter of 50 holes was measured, from which a 3-fold value (3σ) of standard deviation (σ) was computed and reported as CDU. A smaller value of CDU is better.

### Evaluation of MEF

Exposure was made through a mask having a fixed pitch and a varying dot size, scaled as on-wafer size at the optimum dose. The size of the hole pattern transferred to the wafer was measured. With respect to the hole size, the size of the transferred pattern is plotted relative to the mask design size, and a gradient is computed by linear approximation, and reported as MEF. A smaller value of MEF indicates reduced influence of a finish error of the mask pattern and is better.

### Evaluation of DOF

A focus range where a hole pattern could be resolved at the optimum dose was determined and reported as depth of focus (DOF, nm). A larger value of DOF indicates a wider permissible margin for focus offset and is preferable.

The results are shown in Table 8.

TABLE 8

| | | Resist | PEB temp. | Eop | CDU | | DOF |
|---|---|---|---|---|---|---|---|
| | | composition | (° C.) | (mJ/cm²) | (nm) | MEF | (nm) |
| Example | 4-1 | R-01 | 85 | 40 | 4.8 | 4.1 | 80 |
| | 4-2 | R-02 | 85 | 37 | 5.0 | 3.9 | 85 |
| | 4-3 | R-03 | 75 | 41 | 5.0 | 4.0 | 85 |
| | 4-4 | R-04 | 95 | 42 | 5.2 | 3.8 | 80 |
| | 4-5 | R-05 | 85 | 39 | 4.9 | 4.0 | 80 |
| | 4-6 | R-06 | 85 | 39 | 5.0 | 3.9 | 85 |
| | 4-7 | R-07 | 90 | 43 | 5.3 | 3.7 | 73 |
| | 4-8 | R-08 | 90 | 43 | 5.1 | 3.8 | 75 |
| | 4-9 | R-15 | 85 | 44 | 4.7 | 4.1 | 85 |
| | 4-10 | R-16 | 85 | 43 | 4.6 | 4.1 | 85 |
| Comparative | 3-1 | CR-01 | 85 | 40 | 7.2 | 4.8 | 50 |
| Example | 3-2 | CR-02 | 85 | 36 | 6.8 | 5.1 | 55 |
| | 3-3 | CR-03 | 85 | 39 | 7.0 | 5.0 | 45 |

| | | |
|---|---|---|
| 3-4 | CR-04 | Not resolved |

It is evident from the data of Table 8 that the resist compositions within the scope of the invention form, through organic solvent development, negative patterns having improved CDU, MEF and DOF. They are thus best suited for lithography micropatterning. It is noted that no pattern was observed in Comparative Example 3-4. This indicates that Q-4, though used as a quencher, functions as an acid generator.

### [6] EB Lithography Test: Evaluation of L/S Pattern

Examples 5-1 to 5-6 and Comparative Examples 4-1 to 4-4

A silicon substrate was coated with an antireflective coating of 60 nm thick (DUV-62, Nissan Chemical Corp.). Each of the resist compositions (R-09 to R-14, CR-05 to CR-08) was spin coated on the substrate and prebaked on a hotplate at 105° C. for 60 seconds to form a resist film of 50 nm thick. The resist film was exposed to EB using an EB lithography system ELS-F125 (Elionix Co., Ltd., accelerating voltage 125 kV). The resist film was baked (PEB) on a hotplate at the temperature shown in Table 8 for 60 seconds and developed in a 2.38 wt % TMAH aqueous solution for 30 seconds. The resist film in the exposed regions was dissolved in the developer to form a positive resist pattern which was a L/S pattern having a space width of 45 nm and a pitch of 90 nm.

### Evaluation of Sensitivity

The L/S pattern was observed under an electron microscope. As an index of sensitivity, the optimum dose Eop (μC/cm²) which provided a L/S pattern with a space width of 45 nm and a pitch of 90 nm was determined.

### Evaluation of LWR

The L/S pattern formed by exposure in the optimum dose Eop was observed under CD-SEM (CG-5000, Hitachi High-Technologies Corp.). The space width was measured at longitudinally spaced apart 10 points, from which a 3-fold value (3σ) of standard deviation (σ) was determined and reported as LWR. A smaller value of LWR indicates a pattern having a less roughness and uniform space width.

### Evaluation of Profile

The L/S pattern formed by exposure in the optimum dose Eop was observed. The pattern was rated “OK” when the pattern profile was rectangular and the sidewalls were substantially perpendicular, or “NG” when the pattern had a tapered profile with noticeably graded sidewalls or a top rounded profile due to top loss.

The results are shown in Table 9.

TABLE 9

| | | Resist | PEB temp. | Eop | LWR | |
|---|---|---|---|---|---|---|
| | | composition | (° C.) | (μC/cm²) | (nm) | Profile |
| Example | 5-1 | R-09 | 90 | 26 | 4.8 | OK |
| | 5-2 | R-10 | 95 | 24 | 4.6 | OK |
| | 5-3 | R-11 | 85 | 25 | 4.9 | OK |
| | 5-4 | R-12 | 85 | 24 | 4.8 | OK |
| | 5-5 | R-13 | 90 | 24 | 4.9 | OK |
| | 5-6 | R-14 | 90 | 25 | 4.7 | OK |
| Comparative | 4-1 | CR-05 | 90 | 26 | 6.1 | NG |
| Example | 4-2 | CR-06 | 90 | 25 | 5.7 | NG |
| | 4-3 | CR-07 | 95 | 27 | 5.9 | NG |

| | | |
|---|---|---|
| 4-4 | CR-08 | Not resolved |

It is evident from Table 9 that the inventive resist composition is improved in sensitivity, profile (rectangularity), and LWR upon positive pattern formation by EB lithography and alkaline aqueous solution development. In Examples 5-1 to 5-6, EB was used for exposure of the resist films. It is generally known that even when radiation of short wavelength such as EUV is used, similar basic resist properties are obtainable, that is, the EB lithography test and the EUV lithography test are correlated. It is thus estimated that the inventive resist composition is also improved in sensitivity, profile, and LWR when processed by the EUV lithography. It is noted that no pattern was observed in Comparative Example 4-4, which indicates that Q-4, though used as a quencher, functions as an acid generator.

Japanese Patent Application No. 2018-183103 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 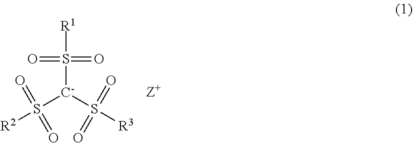
wherein R¹, R² and R³ are each independently a C₁-C₂₀ straight, branched or cyclic monovalent hydrocarbon group which may contain a heteroatom exclusive of fluorine, R² and R³ may bond together to form a ring with the sulfur atoms to which they are attached and the carbon atom in the formula, and Z⁺ is a sulfonium, iodonium or ammonium cation.

2. A resist composition comprising (A) the onium salt of claim 1, (B) an organic solvent, (C) a polymer comprising recurring units containing an acid labile group, and (D) a photoacid generator.

3. The resist composition of claim 2 wherein the recurring units containing an acid labile group have the formula (a1) or (a2): 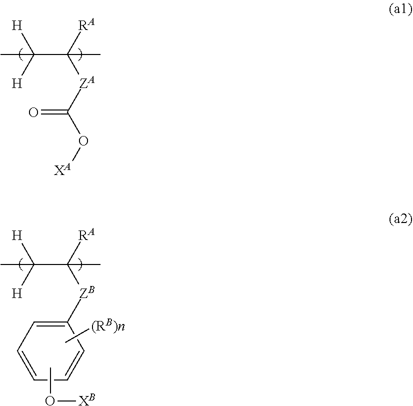
wherein R^{A} is each independently hydrogen, fluorine, methyl or trifluoromethyl, Z^{A} is a single bond, phenylene, naphthylene or (backbone)—C(═O)—O—Z^{A1}—, Z^{A1} is a C₁-C₁₀ straight, branched or cyclic alkanediyl group which may contain a hydroxyl moiety, ether bond, ester bond or lactone ring, or phenylene or naphthylene group, Z^{B} is a single bond or (backbone)—C(═O)—O—, X^{A} and X^{B} are each independently an acid labile group, R^{B} is a C₁-C₂₀ straight, branched or cyclic monovalent hydrocarbon group which may contain a heteroatom, and n is an integer of 0 to 4.

4. The resist composition of claim 2 wherein the polymer further comprises recurring units having the formula (b1) or (b2): 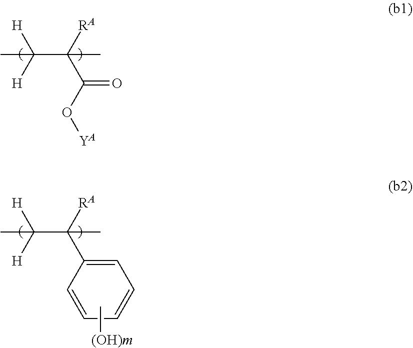
wherein R^{A} is each independently hydrogen, fluorine, methyl or trifluoromethyl, Y^{A} is hydrogen or a polar group containing at least one structure selected from the group consisting of hydroxyl, cyano, carbonyl, carboxyl, ether bond, ester bond, sulfonic acid ester bond, carbonate bond, lactone ring, sultone ring and carboxylic anhydride, and m is 1 or 2.

5. The resist composition of claim 2 wherein the photoacid generator (D) has the formula (2) or (3): 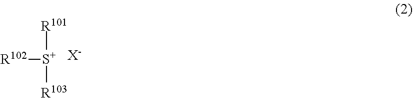
wherein R¹⁰¹, R¹⁰² and R¹⁰³ are each independently a C₁-C₂₀ straight, branched or cyclic monovalent hydrocarbon group which may contain a heteroatom, any two of R¹⁰¹, R¹⁰² and R¹⁰³ may bond together to form a ring with the sulfur atom to which they are attached, and X⁻ is an anion selected from the following formulae (2A) to (2D): 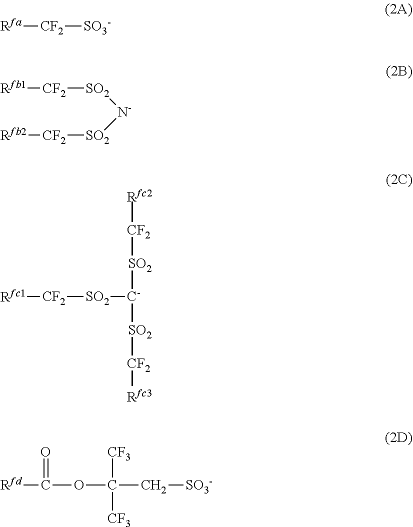
wherein R^{fa}, R^{fb1}, R^{fb2}, R^{fc1}, R^{fc2} and R^{fc3} are each independently fluorine or a C₁-C₄₀ straight, branched or cyclic monovalent hydrocarbon group which may contain a heteroatom, or a pair of R^{fb1} and R^{fb2}, or R^{fc1} and R^{fc2} may bond together to form a ring with the carbon atom to which they are attached and any intervening atoms, R^{fd} is a C₁-C₄₀ straight, branched or cyclic monovalent hydrocarbon group which may contain a heteroatom, 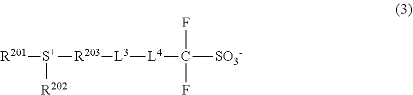
wherein R²⁰¹ and R²⁰² are each independently a C₁-C₂₀ straight, branched or cyclic monovalent hydrocarbon group which may contain a heteroatom, R²⁰³ is a C₁-C₂₀ straight, branched or cyclic divalent hydrocarbon group which may contain a heteroatom, any two of R²⁰¹, R²⁰² and R²⁰³ may bond together to form a ring with the sulfur atom to which they are attached, L³ is a divalent linking group, and L⁴ is a single bond or a C₁-C₂₀ straight, branched or cyclic divalent hydrocarbon group which may contain a heteroatom.

6. The resist composition of claim 2, further comprising (E) a surfactant which is insoluble or substantially insoluble in water and soluble in alkaline developer, and/or a surfactant which is insoluble or substantially insoluble in water and alkaline developer.

7. The resist composition of claim 2, further comprising (F) a nitrogen-containing compound.

8. A pattern forming process comprising the steps of applying the resist composition of claim 2 onto a substrate to form a resist film, exposing the resist film to KrF excimer laser, ArF excimer laser, EB or EUV, and developing the exposed resist film in a developer.
